SEQUENCE LISTING (2 sequences)

SEQ ID NO: 1 - protein, 18 aa, synthetic construct
EGRGSLLTCGDVEENPGP

SEQ ID NO: 2 - protein, 19 aa, synthetic construct
ATNFSLLKQAGDVEENPGP

(19) United States
(12) Patent Application Publication
(10) Pub. No.: US 2026/0297616 A1
(43) Pub. Date: Oct. 1, 2026

(54) CELL LINES AND METHODS FOR PRODUCING RECOMBINANT ADENO-ASSOCIATED VIRUS

(71) Applicant: REGENTS OF THE UNIVERSITY OF MINNESOTA, Minneapolis, MN (US)

(72) Inventors: Min Lu, Belmont, CA (US); Zion Lee, South San Francisco, CA (US); Wei-Shou Hu, Falcon Heights, MN (US); Han-Jung Kuo, St. Paul, MN (US); Yu-Chieh Lin, Minneapolis, MN (US)

(21) Appl. No.: 19/476,922

(22) PCT Filed: Apr. 19, 2024
(86) PCT No.: PCT/US2024/025382

§ 371 Date: Oct. 20, 2025

Related U.S. Application Data
(60) Provisional application No. 63/460,996, filed on Apr. 21, 2023.

(51) Int. Cl. C12N 15/86

(52) U.S. Cl. CPC C12N 15/86; C12N 2750/14143; C12N 2750/14152; C12N 2830/002

(57) ABSTRACT

A stable mammalian cell line includes a first polynucleotide encoding a first AAV inverted terminal repeat (ITR) sequence. In one or more embodiments, the first polynucleotide includes a first inducible promoter. The stable mammalian cell line may further include a second polynucleotide encoding a repressor, wherein the first inducible promoter is operably linked to the repressor. A method of using a stable mammalian cell line includes contacting the stable mammalian cell line with an inducer. A polynucleotide encodes a first AAV inverted terminal repeat (ITR) sequence, a first inducible promoter, and a repressor.

### CROSS-REFERENCE TO RELATED APPLICATION

This application claims the benefit of U.S. Provisional Patent Application No. 63/460,996, filed Apr. 21, 2023, which is incorporated herein by reference in its entirety.

### SEQUENCE LISTING

This application contains a Sequence Listing electronically submitted via Patent Center to the United States Patent and Trademark Office as an XML file entitled “0110000721WO01” having a size of 2,810 bytes and created on Apr. 18, 2024. The information contained in the Sequence Listing is incorporated by reference herein.

### SUMMARY

In one aspect, the present disclosure relates to a stable mammalian cell line including: a first polynucleotide encoding a first AAV inverted terminal repeat (ITR) sequence and a first promoter, wherein the first promoter is not expressed in the stable mammalian cell line. In one or more embodiments, the first promoter is a tissue-specific promoter. In one or more embodiments, the first promoter is a first inducible promoter. In one or more embodiments, the first polynucleotide further includes a second ITR sequence. In one or more of these embodiments, the first promoter is located between the first ITR sequence and the second ITR sequence. In one or more embodiments, the first polynucleotide encodes a gene of interest (GOI).

In one or more embodiments, the stable mammalian cell line includes a second polynucleotide encoding a repressor sequence wherein the first inducible promoter is operably linked to the repressor. In one or more embodiments, the stable mammalian cell line includes a third polynucleotide encoding an AAV large replicate (Rep) protein, wherein the third polynucleotide is operably linked to a second inducible promoter; a fourth polynucleotide encoding an adenovirus (Ad) E4orf6, wherein the fourth polynucleotide is operably linked to a third inducible promoter; and a fifth polynucleotide encoding an Ad DNA binding protein (DBP), wherein the fifth polynucleotide is operably linked to a fourth inducible promoter.

In one or more embodiments, the second inducible promoter, the third inducible promoter, and the fourth inducible promoter have at least 80% sequence identity, are induced by a common stimulus, or both.

In one or more embodiments, the stable mammalian cell line includes a sixth polynucleotide encoding an AAV capsid protein, wherein the sixth polynucleotide is operably linked to a fifth inducible promoter; and a seventh polynucleotide encoding an AAV small Rep protein, wherein the seventh polynucleotide is operably linked to a sixth inducible promoter.

In another aspect, the present disclosure relates to a stable mammalian cell line including: a first polynucleotide encoding an AAV large replicate (Rep) protein, wherein the first polynucleotide is operably linked to a first inducible promoter; a second polynucleotide encoding an adenovirus (Ad) E4orf6, wherein the second polynucleotide is operably linked to a second inducible promoter; a third polynucleotide encoding an Ad DNA binding protein (DBP), wherein the third polynucleotide is operably linked to a third inducible promoter; a fourth polynucleotide encoding an AAV capsid protein, wherein the fourth polynucleotide is operably linked to a fourth inducible promoter; and a fifth polynucleotide encoding an AAV small Rep protein, wherein the fifth polynucleotide is operably linked to a fifth inducible promoter.

In one or more embodiments, the stable mammalian cell line includes a sixth polynucleotide encoding a first AAV inverted terminal repeat (ITR) sequence.

In one or more embodiments, the sixth polynucleotide lacks a promoter.

In one or more embodiments, the first inducible promoter, the second inducible promoter, and the third inducible promoter have at least 80% sequence identity. In one or more embodiments, the first inducible promoter, the second inducible promoter, and the third inducible promoter are induced by a common stimulus, such as doxycycline or cumate.

In one or more embodiments, the stable mammalian cell line includes an exogenous DNA polymerase, such as a DNA-dependent DNA polymerase. In one or more embodiments, the exogenous DNA polymerase is a viral DNA polymerase, such as HSV-1 DNA polymerase.

In another aspect, the present disclosure relates to a method of using any of the stable mammalian cell lines disclosed herein. In one or more embodiments, the method includes contacting the mammalian cell line with an inducer of one or more of the inducible promoters.

In one or more embodiments, the method includes contacting the cell with a predetermined amount of inducer, wherein the predetermined amount of inducer is selected to increase the percentage of full rAAV particles.

In one or more embodiments, the method includes contacting the cell line with more than one inducer, removing the inducer from the cell line, or both

In another aspect, the present disclosure relates to a stable mammalian cell line including: a third polynucleotide encoding an AAV large replicate (Rep) protein, wherein the third polynucleotide is operably linked to a second inducible promoter; a fourth polynucleotide encoding an adenovirus (Ad) E4orf6, wherein the fourth polynucleotide is operably linked to a third inducible promoter; a fifth polynucleotide encoding an Ad DNA binding protein (DBP), wherein the fifth polynucleotide is operably linked to the fourth inducible promoter, wherein the second inducible promoter and the third inducible promoter have at least 80% sequence identity; a sixth polynucleotide encoding an AAV capsid protein, wherein the sixth polynucleotide is operably linked to a fifth inducible promoter; and a seventh polynucleotide encoding an AAV small Rep protein, wherein the seventh polynucleotide is operably linked to a sixth inducible promoter.

In one or more embodiments, a method includes transfecting the cell line with: a first polynucleotide encoding a first AAV inverted terminal repeat (ITR) sequence and a first inducible promoter; and a second polynucleotide encoding a repressor sequence wherein the first inducible promoter is operably linked to the repressor. In one or more embodiments, a method includes infecting the cell line with a vector encoding: a first polynucleotide encoding a first AAV inverted terminal repeat (ITR) sequence and a first inducible promoter; and a second polynucleotide encoding a repressor sequence wherein the first inducible promoter is operably linked to the repressor.

In another aspect, the present disclosure relates to a stable mammalian cell line including: a second polynucleotide encoding a repressor sequence; a third polynucleotide encoding an AAV large replicate (Rep) protein, wherein the third polynucleotide is operably linked to a second inducible promoter; a fourth polynucleotide encoding an adenovirus (Ad) E4orf6, wherein the fourth polynucleotide is operably linked to a third inducible promoter; a fifth polynucleotide encoding an Ad DNA binding protein (DBP), wherein the fifth polynucleotide is operably linked to the third inducible promoter, wherein the second inducible promoter and the third inducible promoter have at least 80% sequence identity; a sixth polynucleotide encoding an AAV capsid protein, wherein the sixth polynucleotide is operably linked to a fifth inducible promoter; and a seventh polynucleotide encoding an AAV small Rep protein, wherein the seventh polynucleotide is operably linked to a sixth inducible promoter.

In one or more embodiments, a method includes transfecting the cell line with one or more polynucleotides.

In another aspect, the present disclosure relates to a method of using the stable mammalian cell line disclosed herein, wherein the method includes: (i) contacting the cell with a second inducer, wherein the second inducer induces expression of the second inducible promoter, contacting the cell with a third inducer, wherein the third inducer induces expression of the third inducible promoter, or a fourth inducer, wherein the fourth inducer induces expression of the fourth inducible promoter; (ii) contacting the cell with a first inducer, wherein the first inducer induces expression of the first inducible promoter. In one or more embodiments, a method includes contacting the cell with the second inducer, the third inducer, and the fourth inducer. In one or more embodiments, the cell is contacted with the first inducer at least 10 hours after the cell is contacted with the second inducer, the third inducer, and/or the fourth inducer.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list

### BRIEF DESCRIPTION OF THE FIGURES

FIG. 1. Schematic showing construction and characterization of GX rAAV2 producer cell lines. Gene modules. Symbols: RSV: the Rous sarcoma virus promoter; LacO: the lactose (lac) operator; rtTA3: the reverse Tet transactivator gene; CymR: the Cym Repressor; DD: the destabilization domain.

FIG. 2. Quantification of production qualities of select clones. (A) Encapsidated vector genome (VG) titers. (B) Transducing (TU) titers. (C) The ratio of VG to TU and capsid to TU as vector quality parameters. (D) Specific rAAV titer in VG/cell at 3 and 33 population doubling. Triple plasmid transfection of HEK293 cells with pAAV-CAG-GFP, pAAV2-RC, and pHelper was included for comparison with GX cell lines. Encapsidated VG titers measured by qPCR, TU titers measured by image analysis using RM4 assay cells, and capsid titers measured by ELISA. Data represent mean and standard deviation of three independent replicates. ns: p>0.05, *:p<0.05, **:p<0.01, ***:p<0.001 as determined by an unpaired, two-tailed, two-sample t-test. Multiple-comparisons t-test was assessed by using a false discovery rate (FDR) of 0.05.

FIG. 3. Quantification of production qualities of select clones. (A) Quantification of VG titers with or without induction of GOI expression by addition of 5 mM of IPTG. (B) Quantification of TU titers with or without induction of GOI expression by addition of 5 mM of IPTG. Encapsidated VG titers measured by qPCR, TU titers measured by image analysis using RM4 assay cells, and capsid titers measured by ELISA. Data represent mean and standard deviation of three independent replicates.

FIG. 4. Quantification of viral production before and after induction. (A) Intracellular copy numbers of GM, RM and PM before induction (gray lines and black solid lines) and GM after 72 hours induction with doxycycline (Dox) and cumate (+, black dashed line). (B) Relative transcript levels of viral genes before induction (solid lines) and after doxycycline and cumate induction for 72 hours (dashed line). rAAV production by GX cell lines was carried out by inducing GX cell lines with 10 g/mL of doxycycline and 90 μg/mL of cumate (10D90C) for 72 hours. Data represent mean and standard deviation of triplicate qPCR wells. ns: p>0.05, *:p<0.05, **:p<0.01, ***:p<0.001 as determined by an unpaired, two-tailed, two-sample t-test. Multiple-comparisons t-test was assessed by using a false discovery rate (FDR) of 0.05.

FIG. 5. Quantification of viral genes and proteins after induction. (A) Transcripts per million of viral genes and GAPDH expressed in Pf3 cells at 96 hours post-induction (hpi) measured by RNA sequencing. (B) Absolute quantification of viral proteins expressed in Pf3 cells at 96 hpi measured by parallel reaction monitoring (PRM).

FIG. 6. A schematic depicting an exemplary of rAAV producer cell line development workflow. HEK293 cells were transfected with three gene modules (i.e., GM, RM, PM, molar ratio=1:1:1) and Leap-In transposase mRNA (ATUM) at a 1:1 DNA:RNA weight ratio. After three days of outgrowth, cells were passaged into media containing 2 μg/mL of puromycin (InvivoGen, San Diego, CA), 200 μg/mL of hygromycin B (MilliporeSigma, Burlington, MA), and 10 μg/mL of blasticidin (InvivoGen, San Diego, CA). After two weeks of selection, single-cell cloning was performed in 96-well plates by limiting dilution with a seeding density of 0.5 cells per well. Once reaching confluence, clones were then transferred to 24-well plates for induction and maintenance. After induction for three days, cells were lysed via freeze-thaw cycles, and crude lysate with rAAV was applied to the RM4 assay cells for productivity screening. High producers were expanded for stock.

FIG. 7. Comparison of clones. (A) 66 clones were isolated and ranked based on their rAAV titers. (B) Histogram of rAAV titers obtained through induction of each cell clone. The top 4 clones were used for further analysis.

FIG. 8. (A) Image of GX2 cells upon induction with 10D90C at 24 hpi. (B) Image of GX2 cells upon induction with 10D90C and 5 mM of IPTG at 24 hpi. (C) VG per cell produced in GX cell lines with or without induction of GOI expression. (D) TU per cell produced in GX cell lines with or without induction of GOI expression. Expression was induced by addition of 5 mM of IPTG. Doxycycline and cumate were added at 10 g/mL and 90 μg/mL, respectively. Data represent mean and standard deviation of three independent replicates. Data represent mean and standard deviation of triplicate qPCR wells. ns: p>0.05, *:p<0.05, **:p<0.01, ***:p<0.001 as determined by an unpaired, two-tailed, two-sample t-test. Multiple-comparisons t-test was assessed by using a false discovery rate (FDR) of 0.05.

FIG. 9. Kinetic profiles of viral transcripts in GX cell lines upon induction. (A) Relative transcript level of viral genes in clone GX1. (B) Relative transcript level of viral genes in clone GX2. (C) Relative transcript level of viral genes in clone GX6. (D) Relative transcript level of viral genes in clone GX7.

FIG. 10. Absolute quantification (AQUA) of viral proteins measured by parallel reaction monitoring (PRM). Protein copy per cell was calculated based on the assumption that total protein from one HEK293 cell is 360 pg. Data represent mean and standard deviation of light-to-heavy ratios of peak area of top three fragment ions with high intensity in ion chromatograms.

FIG. 11. (A) Total intracellular virus genome copies per cell. (B) Assembled capsids per cell. (C) Encapsidated VG per cell. Data represent mean and standard deviation of triplicate qPCR wells or ELISA wells.

FIG. 12. Induction conditions affect rAAV production by GX6 cell line. VG, capsid, and TU titers upon induction with 10D90C for 72 hours, 0.5D90C for 72 hours, or 10D90C for 8 hours and then with doxycycline removed for the remainder of 72 hours. Data represent mean and standard deviation of two or three independent replicates.

FIG. 13. Kinetic profiles of capsids, total intracellular virus genome, VG and TU in the GX6 cell line under three induction conditions. Data represent mean and standard deviation of two or three independent replicates.

FIG. 14. Induction conditions affect rAAV production by GX7 cell line. VG, capsid, and TU titers upon induction with 10D90C for 72 hours, 0.5D90C for 72 hours, or 10D90C for 8 hours and then with doxycycline removed for the remainder of 72 hours. Data represent mean and standard deviation of two or three independent replicates.

FIG. 15. Kinetic profiles of capsids, total intracellular virus genome, VG and TU in the GX7 cell line under three induction conditions. Data represent mean and standard deviation of two or three independent replicates.

FIG. 16. Relative transcript levels of viral genes in the GX6 and GX7 cell lines. Data represent mean and standard deviation of triplicate qPCR wells or mean and standard deviation of GFP-positive cell counts from twelve images. *:p<0.05, **:p<0.01, ***:p<0.001 as determined by a one-way analysis of variance (ANOVA) multiple-comparisons test.

FIG. 17. Screening of induction conditions on the transducing unit titer for cell lines GX1, GX2, GX6, and GX7. Three different cumate (30, 60, 90 μg/mL) and four doxycycline concentrations (0.1, 0.5, 2.5, 10 μg/mL) in conjunction with three different time profiles of doxycycline exposure, were tested. Inducers were added at 0 hours. Doxycycline was present throughout the 72-hour period, removed at 8 hpi or added at 8 hpi by replacing the medium. At 72 hpi, the virus particle was extracted by freeze-thaw method and the transduction unit was assayed using RM4 cells. For GX1 and GX2, the previous induction conditions of 10D90C gave a good titer. While for GX6 and GX7, reducing the level of doxycycline, removing doxycycline at 8 h, or delaying the addition of doxycycline gave higher titers than 10D90C. For further characterization, 72-hour induction at a lower doxycycline level of 0.5D90C was selected. Data represent mean and standard deviation of GFP-positive cell counts from multiple images.

FIG. 18. Comparison of induction conditions with the GX6 cell line. Data of three induction conditions, 10D90C, 0.5D90C, and removal of doxycycline at 8 hpi are shown. Data represent mean and standard deviation of three independent replicates or mean and standard deviation of duplicate ELISA wells.

FIG. 19. Comparison of induction conditions with the GX7 cell line. Data of three induction conditions, 10D90C, 0.5D90C, and removal of doxycycline at 8 hpi are shown. Data represent mean and standard deviation of three independent replicates or mean and standard deviation of duplicate ELISA wells.

FIG. 20. Protein expression in GX6 and GX7 cell lines under the three induction conditions. (A) Western blot showing that 24 hours after induction, VP was visible. Both lower dose of doxycycline and removing doxycycline gave higher levels of VP subunits. (B) GX6 and (C) GA7 induced with 10D90C or 0.5D90C were stained with antibodies against assembled capsid particle (A20) and against non-assembled VP subunits (B1) respectively to quantify cells containing assembled capsid and non-assembled capsid proteins by flow cytometry. Relative intensity was calculated by dividing mean fluorescence intensity (MFI) by MFI of uninduced GX cell lines stained with antibodies mentioned. Assembled capsids and non-assembled VP subunits abundance were much higher under 0.5D90C condition than under 10D90C for both GX6 and GX7. For B and C, data represent mean and standard deviation of intensity relative to the background signal under the uninduced condition. *:p<0.05, **:p<0.01, ***:p<0.001 as determined by a one-way analysis of variance (ANOVA) multiple-comparisons test or an unpaired, two-tailed, two-sample t-test. Multiple-comparisons t-test was assessed by using a false discovery rate (FDR) of 0.05.

FIG. 21. Treatment of proteasome inhibitor MG132 increased viral protein abundance and rAAV titers. AQUA of VP1/2/3, VP1, AAP, Rep68, DBP, GFP capsids and VG per cell in GX cell lines at 72 hpi. with or without MG132. GX1 and GX2 were induced with 10D90C, and GX6 and GX7 were induced with 0.5D90C. Proteasome inhibitor, 1 μM of MG132, were added at 24 hpi for 24 hours before being removed by medium replenishment with fresh induction media (MG132, 1 M, 24 h). Data represent mean and standard deviation of light-to-heavy ratios of peak area of top three fragment ions with high intensity in ion chromatograms. *:p<0.05, **:p<0.01, ***:p<0.001 as determined by an unpaired, two-tailed, two-sample t-test. Multiple-comparisons t-test was assessed by using a false discovery rate (FDR) of 0.05.

FIG. 22. AQUA of capsids and VG per cell in GX cell lines at 72 hpi. with or without MG132. Data represent mean and standard deviation of three independent replicates. *:p<0.05, **:p<0.01, ***:p<0.001 as determined by an unpaired, two-tailed, two-sample t-test. Multiple-comparisons t-test was assessed by using a false discovery rate (FDR) of 0.05.

FIG. 23. Quantification of full particle content of virus produced in cell lines GX1, GX2, GX6, or GX7. (A) Full particle content (%) determined by dividing the VG titer by the capsid titer. (B) Full particle content (%) determined by Cryo-EM analysis. For (A), data represent mean and standard deviation of three independent replicates. For (B), data represent ratio of full to total particles counted by enumerating particles in multiple cryo-EM images. ns: p>0.05, as determined by an unpaired, two-tailed, two-sample t-test. Multiple-comparisons t-test was assessed by using a false discovery rate (FDR) of 0.05.

FIG. 24. Comparison of the effect of proteasome inhibitors on virus production in cell liens GX1, GX2, GX6, or GX7. Cells were first induced under respective optimal induction conditions for 24 hours before proteosome inhibitor was added. Different concentrations of bortezomib, carfilzomib, MG132, and ixazomib were added to GX cells. After incubation for two hours, six hours, or 24 hours, the media was changed to inhibitor-free and further cultured until 72 hpi. The transducing titer was measured using RM4 cells. The inhibitors tested were bortezomib, carfilzomib, ixazomib and MG132. These inhibitors inhibit chymotrypsin-like activity of the β5 subunit of the proteasome. MG132 had some conditions which enhanced TU titer on all four cell lines. Data represent mean and standard deviation of GFP-positive cell counts from multiple images.

FIG. 25. MG132 (10 μM, 6 hours) and MG132 (1 μM, 24 hours) were further evaluated. Both conditions had similar enhancing effect on the rAAV productivity. However, for GX1 and GX7 a higher titer was obtained with MG132 at 1 μM incubating for 24 hours. Data represent mean and standard deviation of three independent replicates. ns: p>0.05, *:p<0.05, **:p<0.01, ***:p<0.001 as determined by an unpaired, two-tailed, two-sample t-test. Multiple-comparisons t-test was assessed by using a false discovery rate (FDR) of 0.05.

FIG. 26. The effect of MG132 on viral protein levels was further evaluated by flowcytometry after staining with fluorescence-labelled antibodies. (A, E) Cells were stained with an antibody to detect VP proteins. (B, F) Cells were stained with an antibody to detect assembled capsids to quantify VP-positive and capsid-positive cells. The addition of MG132 increased the expression of both VP1/2/3 and capsid in both GX2 and GX7 under each's respective optimal induction conditions. (C, G) Cells were stained with an antibody to detect Rep68 proteins were quantified through its linkage to mCherry reporter. (D, H) Quantification of GFP. Data represent mean and standard deviation of intensity relative to the background signal under the uninduced condition.

FIG. 27. Electron microscopic examination and quantification of full particles of the rAAV particles produced using GX2 and GX7 under respective optimal induction conditions with or without MG132. (A-D) Particle dimension and morphology were examined by negative-staining transmission electron microscopy (TEM). (E-H) By Cryo-EM imaging, the difference between full and empty particles could be seen more clearly which provides more accurate calculations of full particle contents. Thus, full and empty particles produced from high producers, GX2 and GX7, were examined using cryo-EM and enumerated. To generate sufficient quantities of rAAV particles, cells were seeded into multiple 150 mm cell culture dishes at a density of 7×10⁶ cells per dish. After one day, GX2 were induced with 10D90C while GX7 were induced with 0.5D90C. For MG132 treatment, MG132 was added to culture at 24 hpi. Virus particles were extracted at 72 hpi using AAVpro Extraction Solution (Takara). The AAV crude lysate was then purified using POROS CaptureSelect AAVX (Thermo Fisher Scientific, Inc., Waltham, MA) affinity chromatography. The numbers of empty and full particles in the Cryo-EM images were counted manually.

FIG. 28. Extending MG132 treatment during rAAV production from 24 hours to 48 hours did not adversely affect the productivity. MG132 removal during induction is not convenient to implement. Incubation for 24 hours was compared to incubation for 48 hours (i.e., without removing MG132 at 48 hpi) with no significant difference. Data represent mean and standard deviation of three independent replicates. ns: p>0.05, as determined by an unpaired, two-tailed, two-sample t-test. Multiple-comparisons t-test was assessed by using a false discovery rate (FDR) of 0.05.

FIG. 29. Correlation or the lack of correlation between different rAAV components. (A) Correlation between VG and TU titer, (B) Correlation between total virus genome and VG titer,

FIG. 30. Correlation or the lack of correlation between different rAAV components (A) Correlation between capsid and VG titer, (B) Correlation between capsid and VP1/2/3 copies per cell,

FIG. 31. Correlation or the lack of correlation between different rAAV components (A) Correlation between DBP protein copies and total virus genome, (B) Correlation between Rep68 protein copies and total virus genome,

FIG. 32. Correlation or the lack of correlation between different rAAV components (A) Correlation between total virus genome and full particle content, and (B) Correlation between capsid titers and full particle content determined by data from different batches of rAAV production using GX cell lines.

FIG. 33. A schematic showing an exemplary selectable marker for selection of cells with a high number of integrated polynucleotide copies.

FIG. 34. Sequential transfection of cells to produce AAV. Around 15% of cells were BFP-positive. After 1 week post transfection, BFP-positive cells were sorted by FACS.

FIG. 35. Higher transducing titers were achieved by increased copy number of PM encoding VP1/2/3 and Rep52 proteins. The ratio of each genetic module plays an important role in rAAV productivity in synthetic cell lines. Manipulation of DNA ratio of each genetic module for transfection is crucial. Introducing more copies of the genetic module specific to limiting factors is an important method for further enhancement.

FIG. 36. Higher VG and capsid titers were achieved by increased copy number of PM encoding VP1/2/3 and Rep52 proteins. A five-fold increase in VG titer and an 80-fold increase in capsid titer were seen by sequential transfection of GX2 with PM-BFP. A 15-fold increase in VG titer and a 600-fold increase in capsid titer were seen by sequential transfection of GX6 with PM-BFP.

FIG. 37. Higher integrated copies of PM and higher levels of VP123 transcript were achieved by sequential transfection of GX2 and GX6 with PM-BFP. Solid line represents copies or transcript levels before induction. Dash line represents copies or transcript levels at 72 hours after Dox and cumate induction. A four-fold increase in integrated copies of PM and a five-fold increase in VP123 transcript level upon Dox and cumate induction were seen by sequential transfection of GX2 with PM-BFP. A 15-fold increase in integrated copies of PM and a 12-fold increase in VP123 transcript level were seen by sequential transfection of GX6 with PM-BFP.

FIG. 38. Isolation and characterization of high-producing cell lines. (A) Encapsidated vector genome (VG) titers. (B) Copy numbers of integrated PM. (C) Capsid titers. (D) The ratio of VG to TU and VG to capsid as vector quality parameters. (E) Absolute quantification (AQUA) of viral proteins measured by mass spectrometry with the parallel reaction monitoring (PRM) method. For (A)-(D), data represent mean and standard deviation of three independent replicates. For E, data represent mean and standard deviation calculated from light to heavy peak area ratios of top three fragment ions in ion chromatograms multiplied by the concentration of peptide standards.

FIG. 39. Induction conditions affected rAAV production by GX6A and GX6B cell lines. (A-D) for GX6A. (E-H) for GX6B. (A, E) Transcript levels of viral genes relative to GAPDH. (B, F) Abundance of viral proteins determined by targeted proteomics. (C, G) Total intracellular virus genomes, VG and capsid titers, and full particle content. (D) Kinetic profiles of capsids, total intracellular virus genome (TG) and VG in GX6A cells induced with 10D10C and (H) GX6B cells induced with 10D90C. For (A) and (E), data represent mean and standard deviation of triplicate qPCR wells. For (B) and (F), data represent mean and standard deviation calculated from light to heavy peak area ratios of top three fragment ions in ion chromatograms multiplied by the concentration of peptide standards. For (C), (D), (G), and (H), data represent mean and standard deviation of three independent replicates.

FIG. 40. Tuning the cumate induction time to modulate capsid formation dynamics and increase full particle content. (A) VG titers, (B) capsid titers, and (C) full particle content obtained through GX6A and GX6B induced with Dox (10 μg/mL) at 0 hour and cumate (90 μg/mL) at different times. (D-E) Kinetic profiles of capsids, total intracellular virus genomes (TG) and VG for (D) GX6A and (E) GX6B cells with and without a 16-hour delay of cumate (90 μg/mL) induction. Data represent mean and standard deviation of three independent replicates. *:p<0.05 and **:p<0.01 as determined by an unpaired, two-tailed, two-sample t-test.

FIG. 41. The specific productivity in TU/cell or VG/cell is stable over time for engineered cell lines. (A) Virus production in GX6A cells. (B) Virus production in GX6B cells.

FIG. 42. Adaptation of GX6B to serum-free suspension culture. The open arrows indicated the time point changing half medium. The black arrows indicated that the time point the cell culture was split into two.

FIG. 43. Suspension-adapted GX6Bs has rAAR productivity comparable to GX6B when reverted to serum-containing and adherent culture. (A) Specific rAAV titer in VG/cell 72 hours after induction. (B) The morphology of cells 48 hours after induction.

FIG. 44. Enhancing rAAV production using small molecule additives. GX2 cells were induced with 10D90C and subsequently treated them with PMA, BX795, H151, C16, compound C, AICAR, or colforsin at 24 hours post induction.

FIG. 45. Genetic Modules for AAV8 stable producer cell line construction. Genome Module (GM) contains cargo gene GFP coding sequence (CDS) flanked by AAV2 inverted terminal repeats (ITRs). Replication Module (RM) contains adenoviral helper E4orf6 and DNA-Binding Protein (DBP) CDS, and AAV2 Rep68 CDS under the control of inducible TetON promoter. rAAV8 Packaging Module A (PM8-A) contains AAV8 intron-less cap gene (VP123) with an inefficient ACG start codon for VP1 and AAV2 Rep52 CDS under the control of inducible CumateSwitch promoter. rtTA3 denotes the reverse Tet transactivator gene; DD denotes the destabilization domain; CymR denotes the Cym Repressor.

FIG. 46. Construction of rAAV8 stable production cell line. (A) A schematic representation of cell line production. The GM, RM and PM8-A were integrated to the genome of HEK293 cells. After antibiotic selection, single cell cloning and rAA8 productivity screening, four rAAV8 production cell clones, VH1-4, were obtained. (B) The left y-axis shows the total vector genome titer per cell, including both the VG harvested from cell lysate and culture media, the right y-axis shows the percent of total VG secreted to culture media. TriX denotes rAAV8 triple plasmid transfection of HEK293 cells.

FIG. 47. Low capsid titer limits rAAV8 productivity in VH clones. The AAV capsid titer was measured using enzyme linked immunosorbent assay (ELISA) and represent by gray squares. The total intracellular AAV genome titer was titrated by quantitative real-time reverse-transcription PCR (qRT-PCR) and represent by black circle.

FIG. 48. Boosting VP proteins and capsid enhance rAAV8 productivity. (A) Schematic representation of PM8-B and the workflow of sequential transfection (SX) of VH cells with PM8-B. (B) The total VG titer of VH1 and VH3 before and after integration of PM8-B. **p<0.01, ***p<0.001 compared with each negative control group (Student's t-test).

FIG. 49. Comparable rAAV8 productivity as triple transfection upon boosting VP production. (A) The integrated copy number of GM, RM and all PM, including PM8-A and PM8-B, in parent VH3 and two derived clones, VH3A and VH3B. (B) The total VG and capsid titer of TriX, VH3, VH3A and VH3B. (C) The total VG titer of VH3A and VH3B before and after 19 cell doublings. *:p<0.05, **:p<0.01, ns: no significant different (p>0.05, Student's t-test).

FIG. 50. Enhanced AAV genome amplification by expression of HSV-1 DNA polymerase and processivity factor. UL30 and UL42 were linked by a self-cleaving peptide F2A, which was further linked to fluorescent reporter mCardinal by a P2A. This segment was placed downstream of constitutive CMV promoter in an expression vector. The vector is named UL vector. The empty vector with mCardinal reporter was used as a negative control. GX6B cells were induced with 10 g/mL (10D) doxycycline and transient-transfected with the UL vector. The total intracellular AAV genome was titrated 72 hours after transfection and induction.

FIG. 51. Increased AAV genome amplification upon expression of HSV-1 UL30 and UL42 in GX6B cells. GX6B cells were transient-transfected with UL expression vector containing HSV-1 UL30 and UL42 and simultaneously induced with doxycycline. Transfection with or mCardinal reporter only vector was used as a negative control. 72 hours after induction and transfection, their TG titers were titrated by qRT-PCR. VCD denotes viable cell density. **p<0.01, ***p<0.001 compared with each negative control group (Student's t-test). FIG. 52. Design of Replication Module II harboring TetOn-driven HSV-1 UL30 and UL42. rtTA3 denotes the reverse Tet transactivator gene; LTR denotes the long terminal repeat.

### DETAILED DESCRIPTION OF ILLUSTRATIVE EMBODIMENTS

This disclosure describes methods, polynucleotides, and stable mammalian cell lines that produce recombinant adeno-associated virus (rAAV). rAAV may be used for gene therapy, however, clinical use often requires production of large amounts of rAAV. The present disclosure presents methods, polynucleotides, and stable mammalian cell lines compatible with efficient production of rAAV.

Adeno-associated virus (AAV) is a commonly used delivery vehicle for gene therapy. Its many advantages include effective in-vivo gene delivery, long-term persistency, different tissue tropism by various serotypes, and high safety. Several rAAV-based products, such as LUXTURNA (Spark Therapeutics, Inc., Philadelphia, PA) for retinal dystrophy, ZOLGENSMA (Novartis Gene Therapeutics, Inc., Bannockburn, IL) for spinal muscular atrophy, and HEMGENIX (CSL Behring LLC, King of Prussia, PA) for hemophilia B have been approved by the FDA in recent years, giving rise to much excitement for new rAAV-based therapies to come. However, a very large dosage of rAAV is often required in clinical applications. Manufacturing large quantities of rAAV can be challenging. Innovations in rAAV vector manufacturing are desired to create a scalable and robust production platform to meet the demand.

AAV is a nonenveloped, capsid virus with a 4.7 kb long single-stranded DNA genome. The AAV genome encodes four replication proteins (rep) and three capsid proteins (cap) and one assembly-activating protein (AAP). Two secondary structures called inverted terminal repeats (ITRs), flank the coding region and serve as a self primers for genome replication. During DNA replication, host cellular DNA polymerases first start leading strand DNA synthesis to turn the AAV genome into a double-stranded replicative form genome. Then, large rep proteins (Rep78/68) bind and nick the ITRs and use their helicase activity to unwind the ITR structure and allow its replication by host DNA polymerase. After ITR synthesis and ITR restoration, cellular DNA polymerases synthesize a new strand and displace the initial strand. This process is called single-strand displacement, which generates a new single-stranded genome and a double-stranded replicative form genome.

AAV is replication-defective in the absence of helper virus such as adenovirus (Ad) or herpes simplex virus (HSV). Many components of Ad are involved in efficient AAV replication. Ad E1A promotes transcription from AAV promoters and Ad DBP increases DNA replication processivity. In the absence of Ad, the host cellular DNA repair complex, MRN, regards the ITR structure of AAV genomes as a double-strand break and binds to it, thus limiting AAV genome replication. The complex of Ad E1b55k and Ad E4orf6 functions as a ubiquitin ligase and targets the MRN complex, among others, for degradation, thus facilitating AAV genome replication. Ad also expresses a non-coding RNA, VA RNA, that can inhibit phosphorylation of eIF2α, a factor involved in translation initiation, thus augmenting AAV proteins expression. These elements from helper virus play important roles in AAV genome replication.

To produce infectious AAV particles, the AAV genomes must be transferred into capsids. For each infectious AAV particle, a single-stranded AAV genome is packaged into a pre-formed capsid. Three cap isoforms, VP1, VP2 and VP3, assemble at a ratio of 1:1:10. VP1 and VP2 share the entire VP3 coding sequence that is a core capsid-forming domain. VP1 has a unique N-terminus with phospholipase A2 domain that is significant for infectious activity. The transcript that encodes the three cap isoforms is referred to herein as the “VP123 transcript.” VP2 shows no essential function for assembly and infection. VP3 proteins form a core with beta-barrels that are interconnected by variable loops presented on the capsid surface. The N-terminus of VP1 and the surface loops of VP3 are involved in serotype specificity and receptor interaction. The assembly activating protein (AAP) is encoded in an alternative reading frame within cap and it localizes capsid proteins to the host cell nucleolus and activates capsid assembly. Finally, the small Rep proteins (Rep52/40) use their helicase activity to insert AAV genomes into assembled capsids.

AAV genome replication is most efficient in the presence of components from adenovirus (Ad) such as DNA-binding protein (DBP) which increases processivity of DNA replication and E1B55K/E4orf6 complex which mediates degradation of host cellular proteins such as the MRN complex and p53.

Current rAAV Production Methods and Challenges

As recombinant AAV (rAAV) preclinical and clinical trials increase, efficient production and manufacturing methods for rAAV have become increasingly important. rAAV can be modified to replace the rep and cap genes in the AAV genome with a gene of interest (GOI). The resulting rAAV genome thus lacks the two essential genes for its replication. rAAV is most commonly produced by triple transfection of HEK293 cells with three plasmids, one containing the rAAV genome and two others providing rep/cap genes and helper viral genes including E2A, E4 and VA RNA in trans respectively. Current production methods can provide up to 10⁵ vector genomes per cell. One of the commonly used AAV production methods is multi-plasmid transfection with adherent or suspension HEK293 cells (a human cell line that already expresses the Ad E1 gene). In this method, rAAV is efficiently produced without infection of wild-type helper virus.

In one exemplary approach, three plasmids are co-transfected into producing cells: one plasmid includes a gene of interest (GOI) flanked by one or more ITRs, another plasmid includes rep/cap genes, and a third plasmid contains Adenovirus E2A, E4, and VA RNA genes. Other production methods use recombinant helper viruses, such as herpes simplex virus and Baculovirus, to infect cells. The infected cells produce rAAV with the rAAV genome received by transduction and the helper function from helper virus, facilitating viral transcription and genome replication. However, these methods face several challenges. Inefficient transfection leads to a low percentage of cells receiving all essential plasmids and requires large amounts of plasmids. Using helper virus results in potential replication-competent Ad or rHSV copurified with AAV. In addition, these processes all require two recombinant helper viruses first be produced. Furthermore, the process leaves residual helper viral DNA and proteins as impurities. The potential of the occurrence of replication-competent AAV (rcAAV) in rAAV products requires additional downstream purification.

To achieve large-scale rAAV production, another approach involves creating stable cells with partial essential elements integrated to avoid using transient transfection or infection. For example, copies of rAAV vector genome and rep/cap genes can be introduced into HeLa cells that successfully produce rAAV upon infection of Ad. Aside from the producer cell line, packaging cell lines (HeLa) integrated with only rep/cap genes were also developed. The infection of hybrid Ad that had the rAAV vector genome in the E1 region provided both the helper function and the rAAV vector genome to generate rAAV. In HEK293 cells, constitutive expression of the E1A gene activates AAV promoters leading to cytotoxicity. To overcome this issue, yet another approach involves using a dual slicing switch to minimize leaky expression of four rep proteins for a HEK293 cell-based packaging cell line. To eliminate the need for infection of Ad, one can use a cell line integrated with inducible E1A-E1B, rAAV vector genome, and endogenous rep/cap, E2, E4, and VA RNA genes. However, even though this cell line produced rAAV in the early passages, it lost stability gradually after several passages. Thus, the cytotoxicity of both rep and helper proteins pose a major obstacle for creating stable cell lines for producing rAAV.

Cell lines and methods relating to a synthetic biology approach to construct HEK293 cell lines that harbor rAAV genome and genetic modules that can be induced to express necessary replication, helper, and structural components to produce rAAV using selective gene transduction are described in International Publication No. WO 2022/020712 A1 (Hu et al.). This approach opened a new paradigm of viral vector production through the creation of stable cell line. Importantly, the quality of rAAV produced can be tuned through control of induction conditions. Disclosed herein are improvements upon this method that increase production of rAAV particles. In one or more embodiments, the methods disclosed herein increase the ratio of full to empty rAAV particles produced. As used herein, a “full” rAAV particle includes a sufficient portion of the rAAV genome to express the GOI; an “empty” rAAV particle may include a portion of an rAAV genome insufficient to express the GOI or may not include any portion of the rAAV genome.

### Cell Lines for Recombinant AAV Production

In one aspect, the present disclosure describes a cell line that may be used for recombinant AAV (rAAV) production. Current methods of rAAV production use either transfection with multiple plasmids or infection with multiple viruses. These methods are often cumbersome and do not scale well.

In one or more embodiments, the cell lines disclosed herein may be used in a manufacturing process that is seed virus-free, helper virus-free, and transfection-free using synthetic elements controlling viral genes in a stable cell line. These cell lines enable production platforms that are dynamically scalable and tunable for production of rAAV independent of serotype or gene of interest (GOI).

In one or more embodiments, the cell line is a mammalian cell line. The mammalian cell line, prior to incorporating one or more of the polynucleotides of this disclosure, may include, for example, HEK293 or HeLa. In one or more embodiments, the cell line is an adherent cell line. In one or more embodiments, the cell line is a nonadherent cell line. The cell line may be selected for the presence in its genome of other helper genes. For example, adenovirus E1 gene is harbored in HEK293 cells. While the focus of this disclosure is mammalian cell lines, other cell lines, such as insect cell lines, are contemplated.

In one or more embodiments, the cell line is a stable cell line. As used herein, a “stable” cell line is one that has been modified to harbor one or more exogenous polynucleotides that are maintained across multiple cell divisions. The one or more exogenous polynucleotides are typically integrated into the cell genome. Any suitable copy of an exogenous polynucleotide may be integrated into the cell genome. In one or more embodiments, the exogenous polynucleotide may be integrated randomly into the genome. In one or more embodiments, the exogenous polynucleotide may be integrated into the genome in a targeted manner.

In one or more embodiments, the cell line includes a polynucleotide encoding a selectable marker. Any suitable selectable marker may be used including, for example, resistance to an antibiotic. Exemplary antibiotics include, for example, puromycin, hygromycin, zeocin, geneticin, blasticidin, etc. In an exemplary embodiment, the selectable marker may include puromycin resistance (PuroR). In one or more embodiments, the polynucleotide encoding a selectable marker may be operably linked to constitutive promoter. Any suitable constitutive promoter may be used. Exemplary constitutive promoters include human phosphoglycerate kinase promoter (PGK), CMV promoter (PCMv), and human elongation factor 1 alpha promoter (P_{EF1α}). In an exemplary embodiment, the constitutive promoter may include P_{EF1α}.

In one or more embodiments, a cell line may include a sortable marker. Selectable markers provide a binary indication of whether or not a cell has received and integrated a copy of a desired polynucleotide. In one or more embodiments, it may be desirable to further select for cells that have received a high number of copies of a desired polynucleotide. Any of the polynucleotides described herein may include a sortable marker, most often a fluorescent protein. Any fluorescent protein described herein may be used as a selectable marker. After integration of a polynucleotide into one or more cells, the cells may be expanded, after which the cells may be sorted, for example, using fluorescence-activated cell sorting (FACS) to select those cells that express the higher levels of the selectable marker. High expression of a selectable marker may indicate a high number of copies of a polynucleotide successfully integrated into a cell. Thus, using a selectable marker may enable selection of clones that not only received a polynucleotide of interest, but that received a high number of copies of a polynucleotide of interest. A schematic of an exemplary selection scheme is shown in FIG. 33.

In one or more embodiments, two of the polynucleotides are operably linked. If operably linked, the polynucleotides may be operably linked to a polynucleotide encoding a self-cleaving peptide. Any suitable self-cleaving peptide may be used including, for example, EGRGSLLTCGDVEENPGP (T2A; SEQ ID NO:1) or ATNFSLLKQAGDVEENPGP (P2A; SEQ ID NO:2). In an exemplary embodiment, the self-cleaving peptide includes T2A. If operably linked, the polynucleotides may be operably linked to the same constitutive promoter.

### Cell Lines Including AAV Genome Module (GM)

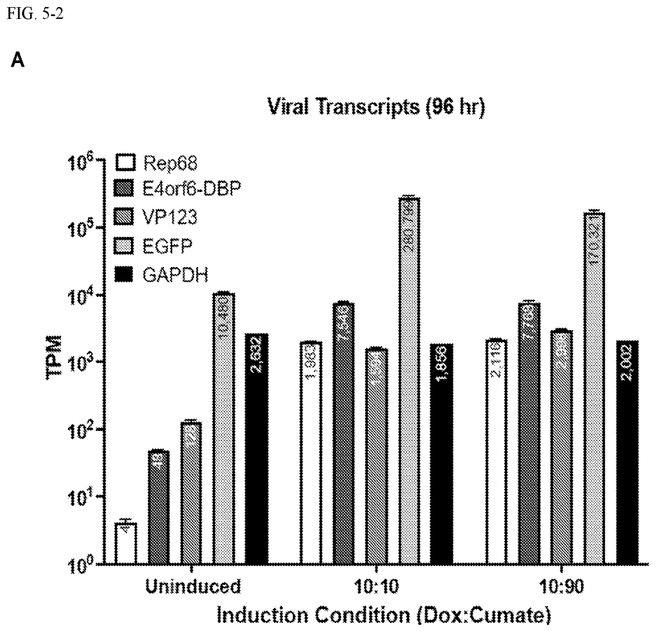

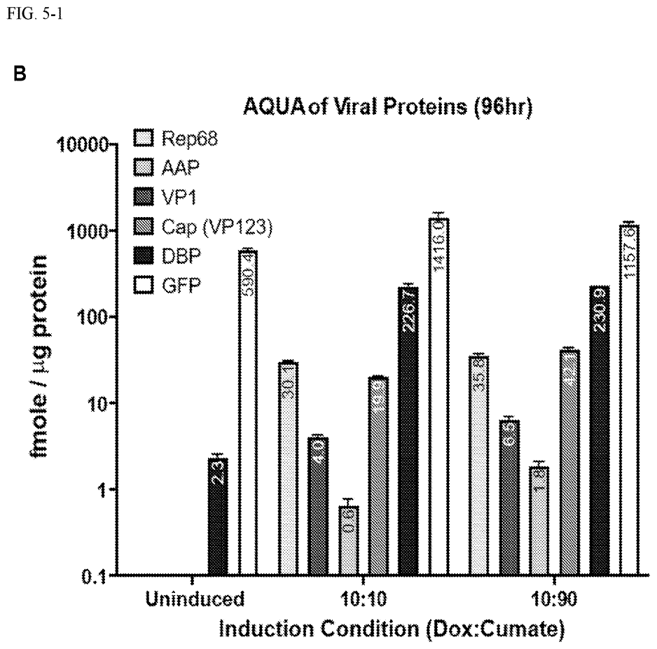

The Pf3 cell line, an HEK293 cell line that had been previously engineered to synthesize rAAV expressing green fluorescence protein (GFP) upon induction had been previously established. Transcriptomic and proteomic analyses of the cell line were used to identify potential limiting factors that could be decreased to enhance rAAV production. Pf3 had a transcript level of GFP several times of GAPDH even under uninduced conditions (FIG. 5). The GFP transcript level went up almost twenty-fold higher upon induction and viral genome amplification accounting for 93% of total viral transcripts or 17% of total cellular mRNAs. In comparison, the VP123 transcript constitutes only 1.64% of viral RNAs or 0.3% of total mRNAs. At protein level, GFP was also much higher than other viral proteins (FIG. 5). Based on the results, such high-level expression of GOI may divert resources for transcription and/or translation to wasteful GOI expression and contributed to the low virus productivity, and postulated that minimizing GOI expression in producing cells would increase rAAV production. Thus, one goal of the present disclosure is to reduce cellular resources used for production of the GOI during rAAV production.

In one or more embodiments, a cell line of the present disclosure includes a first polynucleotide referred to herein as a “genome module” (GM). The GM is generally configured to produce an AAV genome. The AAV genome typically terminates with an inverted terminal repeat (ITR) sequence. An ITR sequence is typically a conserved 145 nucleotide sequence. Multiple ITR sequence variants are known. Most commonly, the ITR sequence from AAV2 or AAV8 is used to produce rAAV. While the ITR sequence may be matched to the capsid protein serotype (e.g., an AAV2 ITR sequence may be used with an AAV2 capsid protein), capsid proteins and ITR serotypes may be non-matching. For example, an AAV3 capsid may package a genome having AAV2 ITRs.

An AAV genome may be single-stranded DNA. An AAV genome may include one or two ITR sequences. An AAV genome produced from a polynucleotide having one ITR typically forms a double-stranded structure via a hairpin fold in the middle. An AAV genome produced from a polynucleotide having two ITR sequences typically forms a single-stranded DNA structure having hairpin folds on each of the 3′ and 5′ termini. The AAV genome may be the positive or negative strand of the polynucleotide from which it is produced.

In one or more embodiments, the GM includes an inducible promoter. The inducible promoter is typically a promoter whose expression is controlled by a repressor or activator. The promoter may be, for example, the Rous sarcoma virus (RSV) promoter, CMV promoter, CAG promoter, PGK promoter, EF1α promoter, SV40 promoter, UBC promoter, SFFV promoter, CBA promoter, CBh promoter, a liver-specific promoter (e.g., hAAT, LP1, TBG, or ALB), or a neuron-specific promoter (e.g., hSYN1, GFAP, DAT, TH, NSE, or CaMKII). Expression of the promoter may be controlled by any suitable inducible system, such as a system including an activator or a repressor. In one or more embodiments, the repressor may be a sequence-targeted repressor system, such as the CRISPR interference (CRISPRi) system. The repressor is typically an element that binds to a region of a polynucleotide to block transcription. In one or more embodiments, the repressor may include a regulator that binds to an operator to suppress transcription.

In one or more embodiments, the activator may an activator system, such as the TetON system. In one or more embodiments, the repressor may be a regulator and operator system. The regulator and operator may be, for example, the lac regulator and operator, the cumate regulator and operator, or the mifepristone repressor and operator, the tetracycline regulator and operator (TRE, Tet response element, also called the Tet-Off system), ecdysone regulator and operator (EcRE), estrogen regulator and operator (ERE), androgen regulator and operator glucocorticoid regulator and operator (GRE), hypoxia regulator and operator (HRE). The regulator-operator can also be like a CRISPRi system with guide RNA guiding a deactivated Cas9 (dCas9) to the region immediately downstream of a GOI's transcriptional start site. The GM additionally typically includes a gene of interest (GOI). The GOI may be any desired gene that fits within the AAV genome. Commonly used GOIs include those encoding fluorescent marker proteins or therapeutically relevant proteins. Exemplary fluorescence marker proteins include, for example, green fluorescent proteins (GFPs) including, for example, enhanced green fluorescent protein (EGFP); GFP-like proteins including, for example, dsRed, eqFP611, Dronpa, TagRFPs, KFP, EosFP/IrisFP, Dendra, etc.; and monomeric red fluorescent proteins (mRFPs) including, for example, mCherry. Exemplary therapeutically relevant proteins include, for example, cystic fibrosis transmembrane conductance regulator (CFTR), factor IX, tumor necrosis factor, alpha-1 antitrypsin, retinoid isomerohydrolase, soluble fins-like tyrosine kinase-1, alpha-sarcoglycan, glutamate decarboxylase, sarco/endoplasmic reticulum Ca²⁺-ATPase, survival of motor neuron 1, nerve growth factor, and tripeptidyl-peptidase 1.

Notably, the combination of promoter and repressor results in conditional expression of the GOI such that the GOI is not expressed during rAAV production. In one or more embodiments, the promoter and repressor results in constitutive expression of the GOI during rAAV infection, e.g., when the rAAV is applied to a cell of interest after it has been produced.

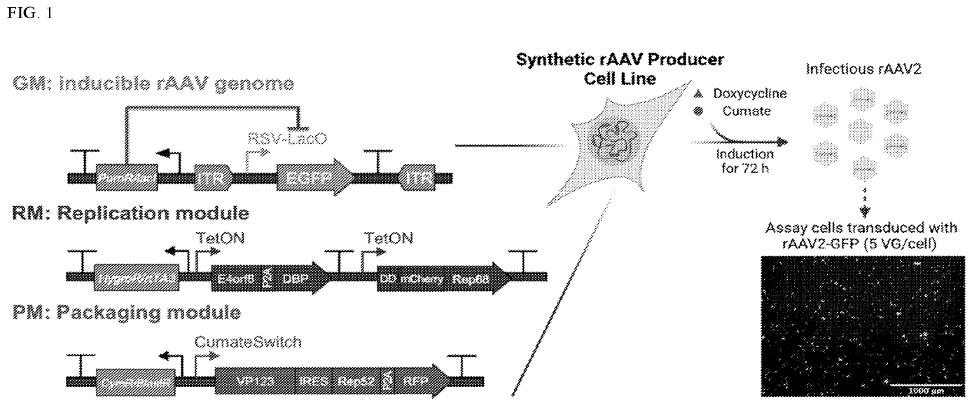

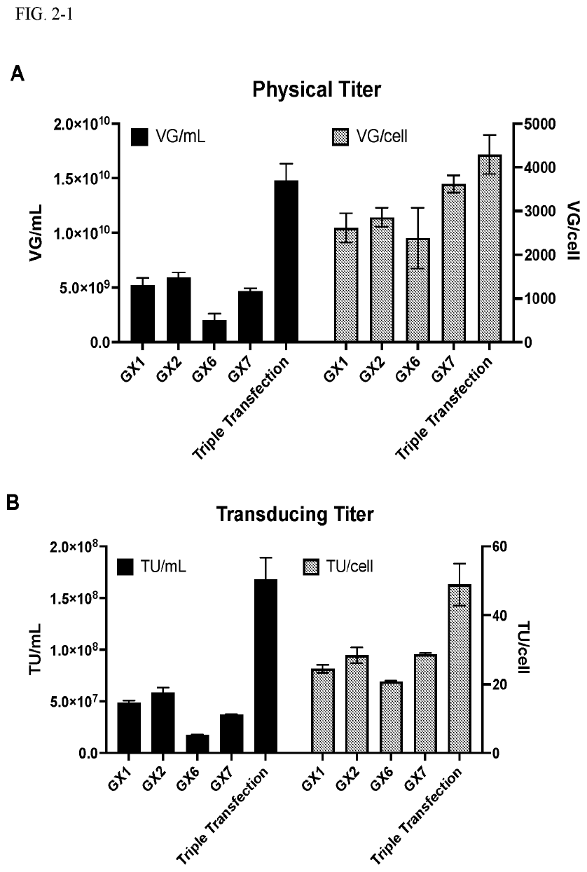

In one illustrative embodiment, the GM includes a GOI, such as GFP and the RSV promoter controlled by the lac operators and repressors (FIG. 1). When the RSV promoter is active, GFP is transcribed. To prepare this GM, the IPTG-inducible lac repressor gene, lacI, was placed upstream of AAV ITR and linked to a puromycin resistance gene via a 2A self-cleaving peptide. Under uninduced conditions, lac is expressed and suppresses GFP expression. The replicated and encapsidated genome does not have lacI gene. Upon transducing into a lacI-free recipient cell, GFP can be expressed.

### Cell Lines Including AAV Replication Module (RM)

In one or more embodiments, the cell line includes a third polynucleotide encoding an adeno-associated virus (AAV) large replicase (Rep) protein, a fourth polynucleotide encoding an adenovirus (Ad) E4orf6, and a fifth polynucleotide encoding an Ad DNA binding protein (DBP). Each of the fourth, fifth, and sixth polynucleotides is operably linked to a promoter. In one or more embodiments, the promoter may preferably be an exogenous promoter (e.g., a non-AAV promoter). In one or more certain embodiments, the promoter may be the same promoter, e.g., each of the polynucleotides includes a copy of the same promoter.

In one or more embodiments, a cell line of the present disclosure includes a replication module (RM). The RM may include the first, second, and third polynucleotides described above. RMs have been previously described, however, an RM of the present disclosure is distinct in that expression of the first, second, and third polynucleotides are induced by the same stimulus. In one or more embodiments, the first, second, and third polynucleotides are each operably linked to the same promoter sequence. In one or more embodiments, the RM includes a conditional promoter, such as the TetON system, the cumate operator/repressor system, the mifepristone-induced GENESWITCH system (Thermo Fisher Scientific, Inc., Waltham, MA), the lac operator/repressor system, a hormone-inducible system (e.g., an ecdysone-inducible expression system), or a rapamycin-inducible system (e.g., the IDIMERIZE regulated transcription system (Takara Bio USA, Inc., San Jose, CA).

In one particular embodiment, the TetON inducible promoter is used to control both Rep68 and helper genes to reduce the size of RM.

In one or more embodiments, the cell line is preferably a stable cell line (as opposed to a transiently transfected cell line). That is, each of the first, second, and third polynucleotides are integrated into the genome of the cell line and expression of the first, second, and third polynucleotides is not lost during cell culture.

The Ad E4orf6 and/or DBP may be derived from any suitable adenovirus or combination of adenoviruses. In one or more embodiments, adenovirus type 2 (Ad2) or adenovirus type 5 (Ad5) may be preferred. In an exemplary embodiment, the Ad E4orf6 may include Ad2 E4orf6. In an exemplary embodiment, the Ad DBP may include Ad2 DBP.

As noted above, the first polynucleotide encodes a large Rep protein. The large Rep proteins include Rep68 and Rep78. In one or more embodiments, the large Rep protein may preferably be Rep68.

In one or more embodiments, the first polynucleotide may be operably linked to a destabilizing domain (DD). Any suitable destabilizing domain may be used. Exemplary destabilizing domains include an FK506-binding protein 12 (FKBP12) DD, a CMP8/4-OHT-estrogen receptor destabilized domain (ER50 DD), a trimethoprim (TMP)-Escherichia coli dihydrofolate reductase (DHFR) DD, and combinations and mutants thereof. In one or more embodiments, the DD may include an FKBP12 or a mutant FKBP12. An exemplary mutant FKBP12 destabilization domain is one that is responsive to Shield-1 ligand. Shield1 stabilizes proteins tagged with a mutated FKBP12-derived destabilization domain (DD) used in PROTEOTUNER systems (Takara Bio, Inc., Shiga, Japan). It is used to protect DD-tagged proteins from proteasomal degradation, resulting in rapid accumulation of the protein.

In one or more embodiments, the first polynucleotide may be operably linked a polynucleotide encoding a marker and/or a marker protein. The marker protein may include a fluorescent marker. In an exemplary embodiment, the marker protein may include mCherry.

### Cell Lines Including a Packaging Module (PM)

In one or more embodiments, the cell line includes a polynucleotide encoding an AAV capsid protein. The AAV capsid protein may be operably linked to a promoter. The AAV capsid protein may include any protein encoded by the AAV cap gene including VP1, VP2, VP3, assembly-activating protein (AAP), or membrane-associated accessory protein (MAAP), or a mutant thereof, or a combination thereof. That is, the AAV capsid protein is not necessarily limited to those proteins that make up the AAV capsid (VP1, VP2, and VP3). In one or more embodiments, the eighth polynucleotide may include the AAV cap gene. Any suitable AAV cap gene may be used. In one or more embodiments, the cap gene may include the serotypes DJ, 2, and 8. The DJ serotype is a synthetic serotype with a chimeric capsid of AAV-2, 4, 5, 8, 9, Avian AAV, Bovine AAV, and Caprine AAV.

In one or more embodiments, the AAV capsid protein may be operably linked to an inducible promoter. Any inducible promoter described herein may be used. In one or more certain embodiments, the AAV capsid protein may be operably linked to a cumate inducible promoter.

In one or more embodiments, the cell line includes a polynucleotide encoding a small Rep protein. The small Rep proteins include Rep40 and Rep52. In one or more embodiments, the small Rep protein may preferably be Rep52.

In one or more embodiments, the cell line includes a packaging module (PM), including a polynucleotide encoding a small Rep protein and a polynucleotide including an AAV capsid protein.

A cell line described herein may include any combination of the polynucleotides described herein. In one or more embodiments, a cell line includes the third, fourth, fifth, sixth, and seventh polynucleotides as described herein. In such a cell line, the third and fourth inducible promoters may be the same. In one or more of these embodiments, the cell line may be transiently transfected with a polynucleotide including the first and second polynucleotides. In one or more embodiments, a cell line includes the second, third, fourth, fifth, sixth, and seventh polynucleotides as described herein. In one or more of these embodiments, the cell line may be transiently transfected with a polynucleotide including the first polynucleotide.

In one or more embodiments, a cell line includes a GM, a PM, and an RM. Because each module is typically integrated into the cell independently, the number of copies of each of the GM, PM, and RM may be different in each stable cell. In one or more embodiments, a cell line includes a roughly equal ratio of the number of copies of GM to PM to RM. In one or more embodiments, a cell line includes more copies of a PM than either a GM or a RM.

As is described in Example 2 of the present disclosure, increasing the number of PM copies in a cell may advantageously increase rAAV production. Multiple strategies may be used to achieve an increased number of PM copies. In one or more embodiments, a cell line includes more than one PM. Typically, each PM is integrated into the genome of the cell. In one or more embodiments, the cell line includes a single PM that has been integrated in the cell across multiple rounds of integration. Typically, each PM in a cell includes capsid proteins from the same serotype of AAV.

### Methods of Producing Cell Lines for Recombinant AAV Production

The components of a cell line for recombinant AAV production may be incorporated into a genome of a cell by any suitable means. In one or more embodiments, the cell is preferably a mammalian cell. The cell line may be selected for the presence in its genome of other helper genes. For example, adenovirus E1 gene is harbored in HEK293 cells.

In one or more embodiments, the components are preferably stably incorporated into a genome of a cell of a cell line.

For example, at least a first polynucleotide encoding a first AAV ITR sequence and a first inducible promoter, a second polynucleotide encoding a repressor sequence, a third polynucleotide encoding an adeno-associated virus (AAV) large replicase (Rep) protein, a fourth polynucleotide encoding an adenovirus (Ad) E4orf6, and a fifth polynucleotide encoding an Ad DNA binding protein (DBP), a sixth polynucleotide encoding an AAV capsid protein, and a seventh polynucleotide encoding an AAV small Rep protein may be stably incorporated into the cell. In one or more embodiments, as further described above, an eighth polynucleotide encoding a regulator may also be stably incorporated into the genome of the cell. In one or more embodiments, as further described above, a ninth polynucleotide encoding a selectable marker may also be stably incorporated into the cell. In one or more embodiments, as further described above, the polynucleotides may be included in modules, including, for example, a Genome Module, a Replication Module, and/or a Packaging Module.

The components may be stably integrated into the genome of the cell by any suitable means. Exemplary means include randomly integrating one or more of the components into the genome of the mammalian cell, integrating one or more of the components into the genome of the mammalian cell using CRISPR, integrating one or more of the components into the genome of the mammalian cell using a transposase, and/or integrating one or more of the components into the genome of the mammalian cell using a lentivirus. Some of the components may be integrated using one method or a combination of methods while other components are integrated using another method or combination of methods.

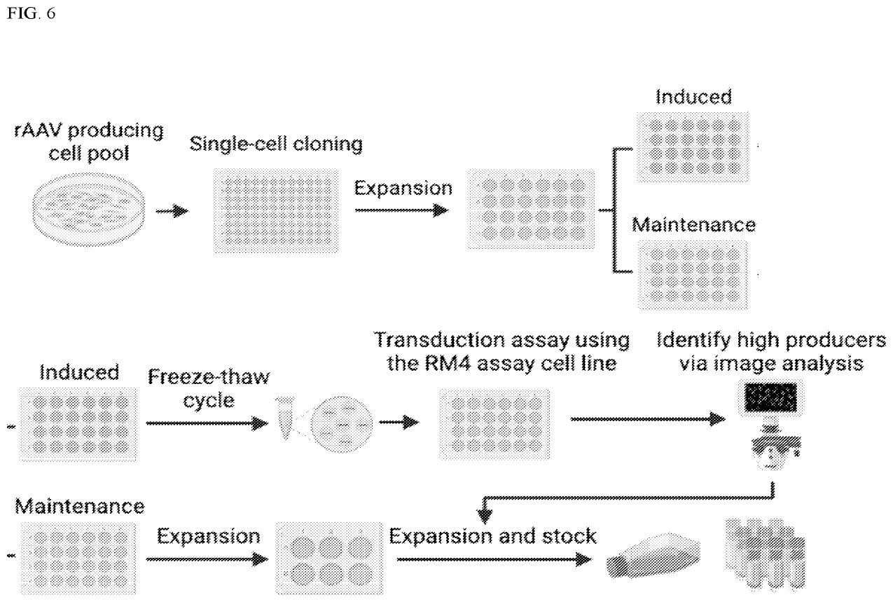

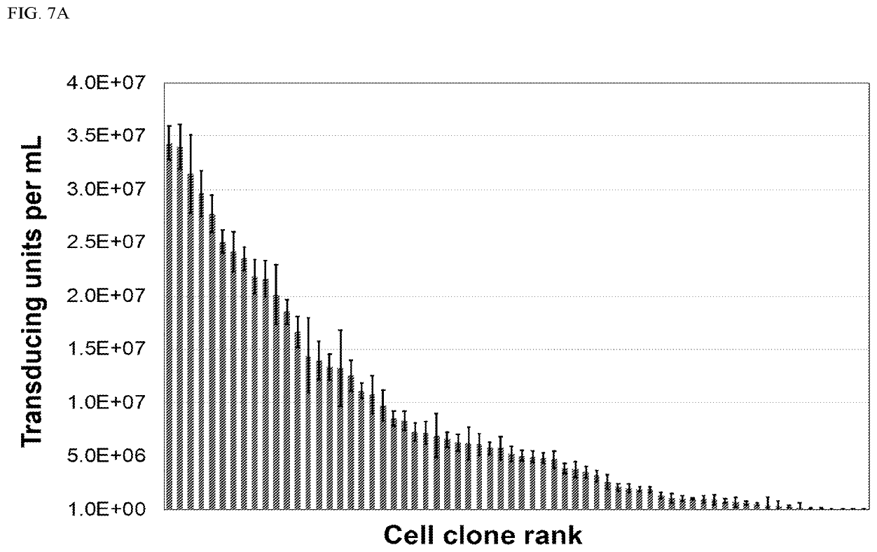

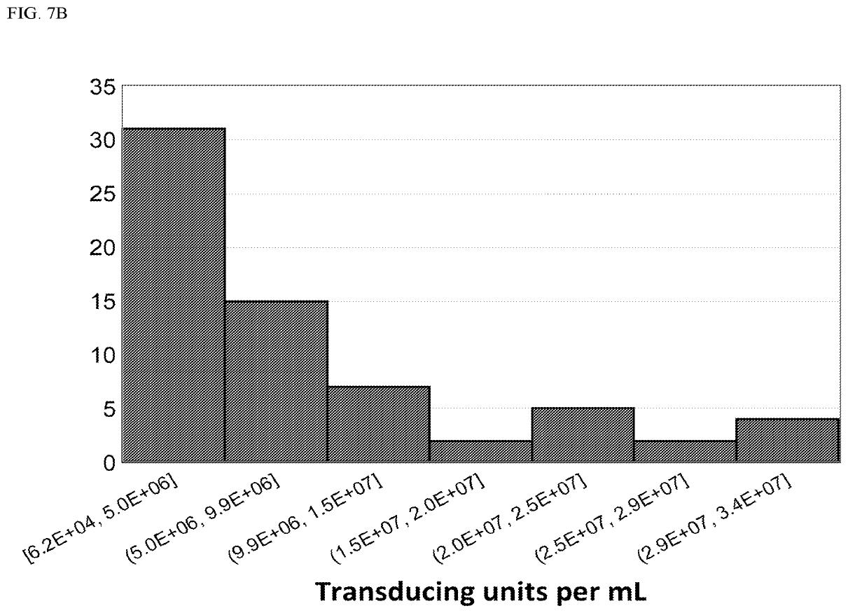

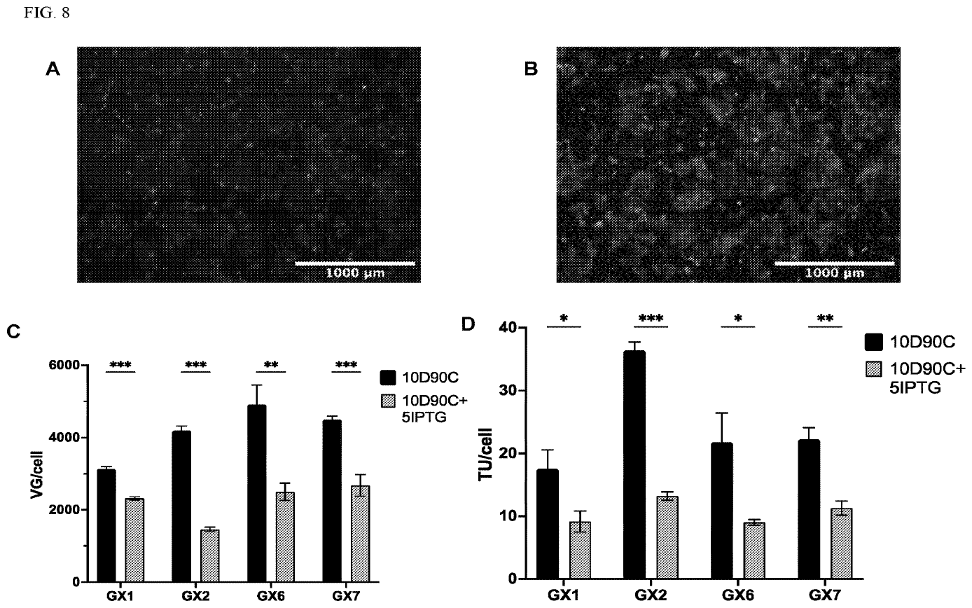

In one particular embodiment, HEK293 cells were transfected with the inducible GM, RM, PM, and transposase mRNA. The GM, RM, and PM sequences integrated randomly within the genome by the transposase. The GM included a polynucleotide encoding puromycin resistance, the RM included a polynucleotide encoding hygromycin resistance, and the PM included a polynucleotide encoding blasticidin resistance. Cells were selected with puromycin, hygromycin and blasticidin for two weeks followed by single-cell cloning. Over sixty clones were screened for high rAAV titer using RM4 assay cells upon induction with 10 μg/mL of doxycycline (Dox) and 90 μg/mL of cumate (10D90C)(FIG. 6, FIG. 7). The top four rAAV producing clones (GX1, GX2, GX6 and GX7) were isolated, expanded and further characterized.

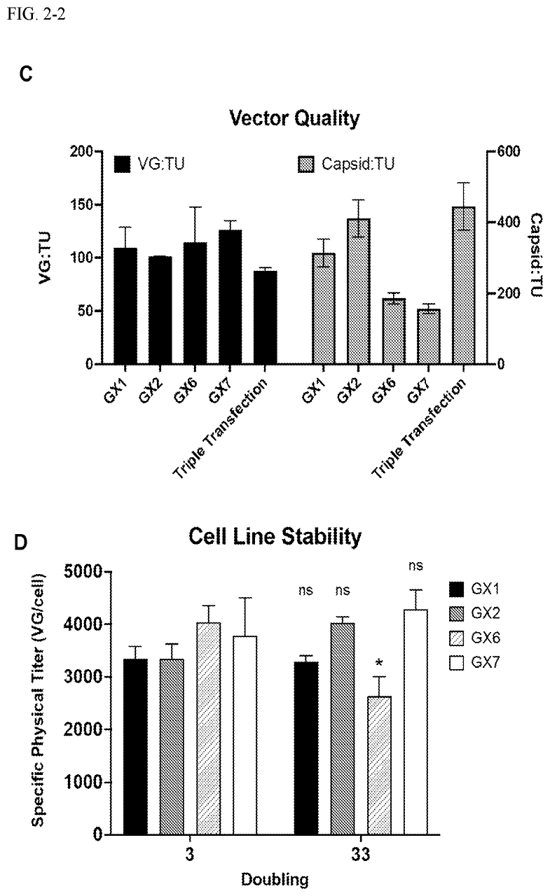

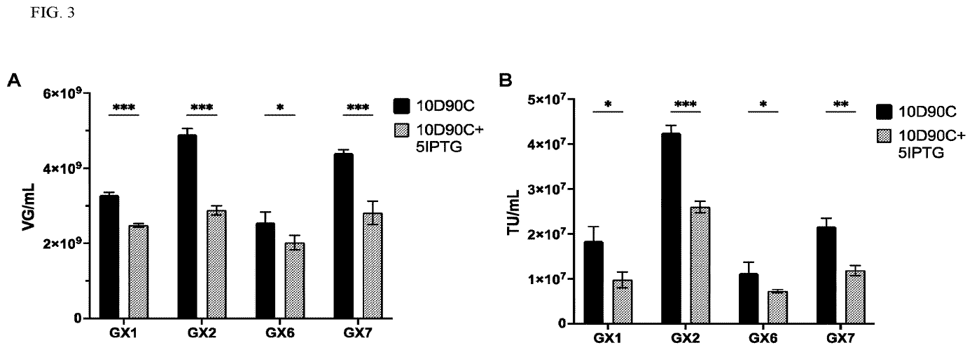

All four clones produced around 5×10⁹ encapsidated vector genomes (VG) per mL or 3,000 VG per cell (FIG. 2) upon induction by doxycycline and cumate. A parallel triple transfection using the traditional pAAV-GFP, pAAV-RC2, and pHelper vectors yielded a somewhat higher titer. On a transducing titer basis (assayed using RM4 assay cells and called transducing units (TU)), the transduction unit largely followed the trend of physical titer (i.e., VG titer) for all four clones (FIG. 2). The VG:TU ratio was around 100:1, and the full particle content estimated by VG to capsid ratio, was up to 81% (FIG. 2). In general, the quality of viral vectors produced by the four clones were similar. Three out of four clones kept the same productivity after 30 doubling (FIG. 2). The effect of inducing GOI expression on the rAAV productivity was evaluated. The rep, helper and cap genes were induced with or without IPTG induction of GFP expression. With 5 mM of IPTG, producer cells became much greener, and both VG and TU titers markedly decreased in all four clones (FIG. 3, FIG. 8). The result indicated that high level of GOI expression imped rAAV production.

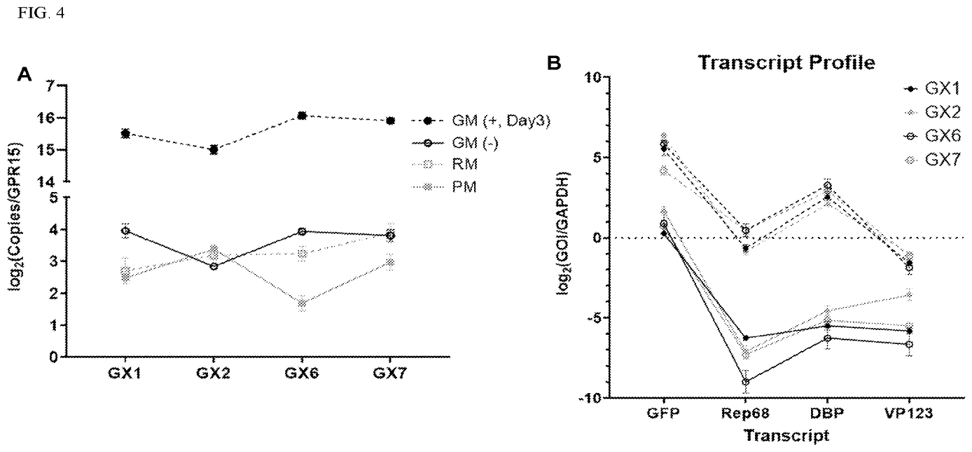

In one particular embodiment, four identified cell line clones were used to produce rAAV. The number of copies of each module integrated into host cell genome, that of amplified viral genome after induction for 72 hours, and the transcript level of viral genes were determined via quantitative PCR (qPCR). The three modules were integrated into the genome in somewhat different numbers (FIG. 4). All four clones had four to eight times more GM integrated than the Pf3 cell line. Induction at 10D90C caused rAAV genome to replicate to reach more than 10⁵ copies per cell. Although the number of cell clones were small, there appears to be a correlation between copy number of integrated GM to total number of amplified genomes. The genes encoded in RM and PM modules had low levels of leaky expression, but GFP in GM was expressed higher than GAPDH (FIG. 4). Upon induction all viral transcripts became highly expressed. High levels of GFP were detected despite the fact that its promoter was uninduced. Without wishing to be bound by theory, this is believed to be because the increased number of rAAV genomes produced during rAAV production exhausted the lac repressor protein.

### Notable Features of GX Lines

Two notable features of the cell lines described herein are the integration of only minimum essential viral elements in host cell genome and replacing native viral transcription regulation elements with inducible promoters whose expression can be tuned through exogenous chemical signals. In the current study, undesirable expression of GOI was reduced, new clones with higher copy number of genetic modules were isolated, induction profile was optimized, and proteasomal degradation of capsid protein was minimized, further advancing productivity. With a strong constitutive promoter driving expression of the GOI, the transcript level of the GOI could reach several times the expression of GAPDH, outcompeting for translation machinery with essentially all other transcript and diverting resources for virus production.

Using an inducible promoter for GOI, the high producing GX cell lines isolated indeed had higher number of copies of GM integrated into host genome than the previous Pf cell lines (FIG. 4). After genome amplification by induction with doxycycline and cumate, the transcript level was still relatively high. With this proof-of-concept design of GM, viral genome replication amplified only ITR-flanked region, but not the lac repressor gene outside the ITR. Hence, after viral genome amplification the vastly large number of LacO outnumbered LacI repressor proteins and lead to an increased transcript level of GOI. Further suppression of GOI, for example by using tightly regulated tissue-specific promoters that have been successfully employed in rAAV targeting different tissues such as muscle, central neuron system, heart and liver for GOI expression, may further enhance the productivity.

### GX Cell Line Profiles

All four GX cell lines had higher rAAV titer than the previous Pf lines. Common features of four GX lines were compared to Pf cells to glean possible features which might contribute to the higher productivity. All four GX cell lines had higher copy numbers of GM and RM than Pf lines. Upon induction and genome amplification, the difference between GX lines and Pf in total genome widened to more than 10-200-fold. This might have contributed to the higher VG titers in GX cells. Some common patterns in transcript level of viral genes were seen. DBP transcript and protein levels were very high in all four cell lines. Rep68 and VP123 transcript and protein levels were several-fold lower than DBP, and VP1 and AAP had the lowest levels. However, there was noticeable difference in Rep68 and VP1/2/3 protein levels and their resulting rAAV products in each clone. At protein level, although GX cells have higher genome copies, GFP protein was lower as attributed to the use of an inducible promoter for GFP.

In one or more embodiments, a cell line includes an additional module to express a polymerase, such as a DNA polymerase. As is described herein, increasing the number of vector genomes in a cell, particularly relative to the number of capsid proteins, may increase the titer of virus produced by the cell. Recombinant virus production typically relies on host cell replication machinery, such as polymerases. However, supplementing a cell with additional replication machinery may increase production of recombinant virus components.

In one or more embodiments, a cell line includes a nucleic acid encoding a polymerase, such as a DNA polymerase. The DNA polymerase may be a viral DNA polymerase. In one or more embodiments, the DNA polymerase is herpes simplex virus-1 (HSV-1) DNA polymerase. In one or more embodiments, the DNA polymerase may include the catalytic subunits and/or the accessory subunits from other herpesviruses. For example, the DNA polymerase may include subunits derived from human cytomegalovirus (HCMV) UL54 and UL44, Epstein-Barr virus (EBV) BALF5 and BMRF1, or Kaposi's sarcoma-associated herpesvirus (KSHV) Pol-8 and PF-8.

### Polynucleotides and Methods of Using Polynucleotides

In one aspect, the present disclosure describes polynucleotides for use in the production of rAAV. While described in the context of exemplary stable cell lines, one or more of the polynucleotides described herein may alternately be transiently added to a cell line. As such, one or more of the polynucleotides described herein may be added to a cell such that the polynucleotide is not integrated into the genome of the cell. Such polynucleotides may be introduced into the cell line in any suitable method, such as transfection or infection.

In one or more embodiments, the polynucleotides described herein may be transfected into a cell line. Transfection may be accomplished using any suitable method known to the art, such as physical transfection, such as electroporation or microinjection, or chemical transfection, such as calcium phosphate-based transfection, or transfection with cationic liposomes or cationic polymers. In one or more embodiments, the polynucleotides described herein may be introduced to a cell line via infection. Infection typically refers to gene transfer using a vector, such as a virus. Suitable vectors include baculovirus, adenovirus, herpesvirus, rabies virus, or retrovirus, such as lentivirus.

Methods of Producing rAAV

In one aspect, the present disclosure describes methods for using the cell lines described herein. In one or more embodiments, a method may enhance rAAV production in synthetic cell lines. The cell lines may be described through multi-omics guided re-design of genetic modules and tuning of viral gene expression. The method can be readily applied to rAAVs of different serotypes with different GOI.

In one or more embodiments, a method of producing rAAV includes treating a cell line of the present disclosure with one or more molecules to induce gene expression. Depending on the number of inducible promoters integrated into the cell line, a different number of molecules may be used to induce gene expression. For example, if a cell line includes polynucleotides encoding three different inducible promoters, treating the cell line to induce gene expression may include treating with three molecules.

rAAV is often grown in adherent cell culture, often adherent HEK293 cell culture. However, suspension culture may also be used to produce larger volumes of rAAV. In one or more embodiments, a method of producing rAAV includes suspension cell culture. In one or more embodiments, a method of producing rAAV includes adherent cell culture.

Transitioning cells to suspension culture often significantly changes the growth characteristics of the cell, including recombinant virus production. As is described in Example 4, transitioning cells from adherent culture to suspension culture may decrease rAAV production. To remedy this loss, the present disclosure describes that one or more modifications may be made to culture conditions to increase rAAV production.

In one or more embodiments, a method includes culturing an rAAV producing cell in suspension. In one or more such embodiments, the method includes culturing the cell in media including serum, such as fetal bovine serum (FBS). A media including serum may be, for example, DMEM with 10% FBS. Additional media components, such as antibiotics, supplementary amino acids, and additional sugars, may be added to the media as well.

In one or more embodiments, suspension culture includes culturing cells on a shaker, such as an orbital shaker. In one or more embodiments, suspension culture includes culturing cells in stirred media, such as in a bioreactor.

In one or more embodiments, a cell line may be adapted from adherent culture to suspension culture. In one or more embodiments, a cell line may be transitioned from suspension culture to adherent culture. As is described herein, transitioning a cell from adherent to suspension culture typically decreases production of virus and recombinant virus such as rAAV. In one or more embodiments, a method includes transitioning a cell between suspension culture and adherent culture. For example, a cell of the present disclosure may be grown in adherent culture in small volume reactors or flask, then subsequently transitioned to suspension culture for high-volume expansion of cells. In one or more embodiments, a method may further include reversion to adherent culture for the induction and production of rAAV. rAAV productivity is typically higher in adherent culture, therefore, employing adherent culture methods to produce recombinant virus may increase yield relative to suspension culture methods.

Methods of Tuning Gene Expression for rAAV Production

In one aspect, this disclosure describes that induction parameters may be modified to improve rAAV production. For example, the concentration of a molecule applied to a cell to induce induction may be modified or predetermined. In one or more embodiments, a method includes contacting a cell with one or more molecules to induce gene expression and later removing the one or more molecules. In one or more embodiments, a method of producing rAAV includes treating a cell line with one or more molecules to induce gene expression for a predetermined amount of time. In embodiments wherein

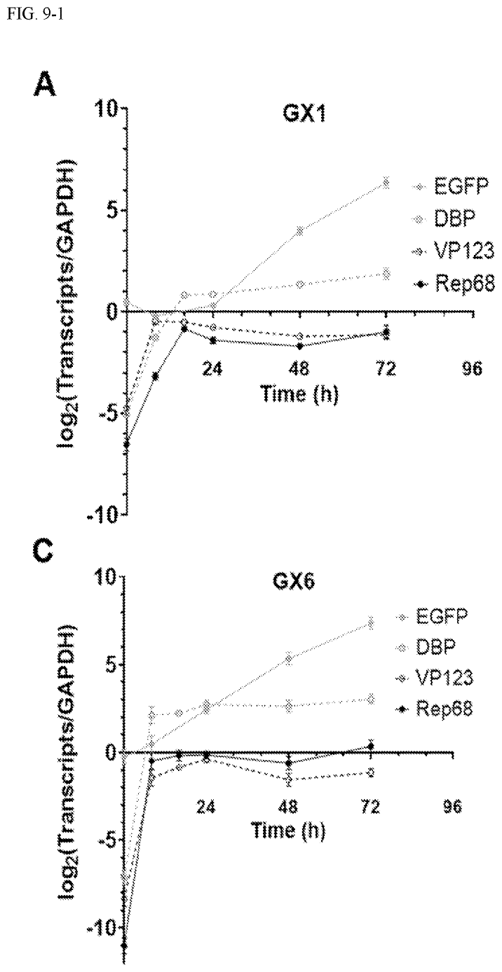

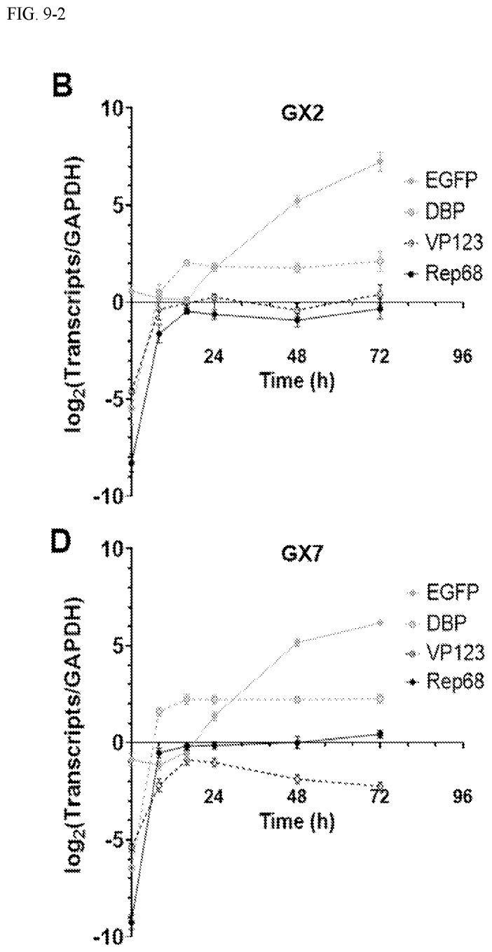

In one or more embodiment, the expression profile of one or more genes may be characterized to select desired parameters for inducing expression of one or more genes. In one example, the time-course profiles of various viral components after induction was characterized. The transcript level of three viral genes, Rep68 and DBP in RM and VP123 in PM, increased rapidly upon induction with doxycycline and cumate and reached close to GAPDH level by 8 hours before leveling off around 16 hours in all four cell lines (FIG. 9). Consistent with the results of FIG. 4, DBP was the most highly expressed viral gene in all four cell lines. The transcript levels of Rep68 and VP123 were several folds lower but still at high levels similar to GAPDH. GFP transcript followed different dynamics to the viral genes, starting from a high leaky expression level and increasing as the viral genome replicated despite absence of inducer IPTG.

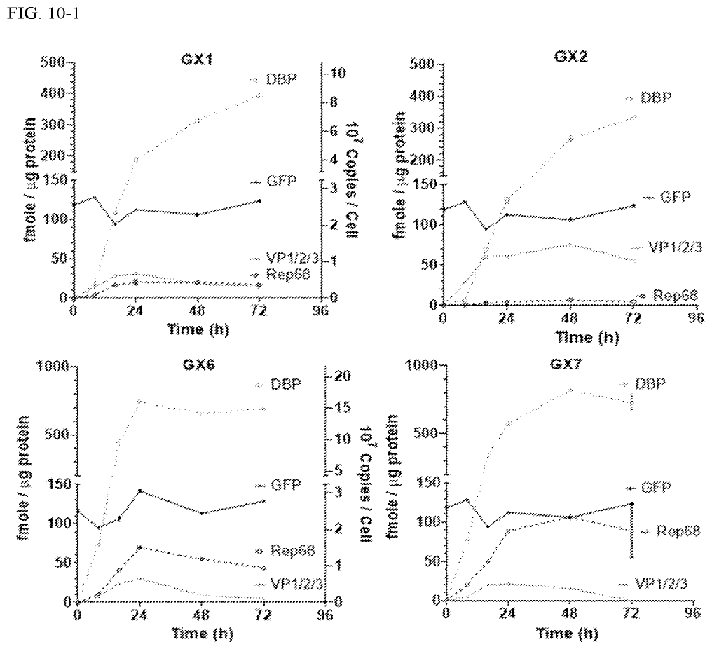

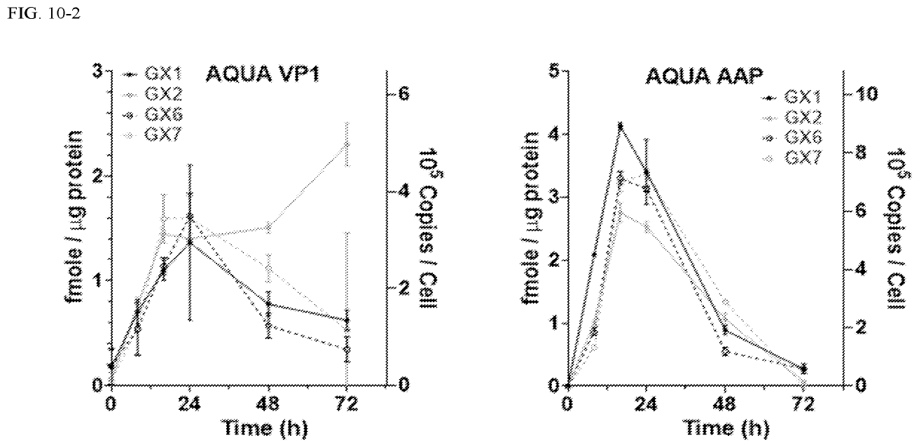

Proteins were identified using targeted quantitative proteomics analysis of heavy isotope-labeled peptide spiked samples. Protein abundance levels fell into two broad groups: Rep68, DBP, VP1/2/3 (quantified using a peptide common to VP1, VP2 and VP3), and GFP were identified on the order of 100 fmol/μg protein (FIG. 10). In contrast, VP1 (quantified using a peptide unique to VP1) and AAP were identified at a level of approximately 5 fmol/μg protein (FIG. 10). The rise of viral proteins lagged somewhat from the corresponding transcript, reaching steady levels around 24 hours. The relative abundance level and dynamics of different proteins followed similar trends in each clone. The most abundant protein was DBP which had more than 10⁷ copies per cell. Notably, VP1 and AAP proteins decreased sharply after peaking around 24 hours, along with similarly but less drastically decrease of VP1/2/3. DBP was the only detectable viral protein in uninduced conditions which had around 5×10⁴ copies per cell. In comparison, beta actin (a protein commonly used as a baseline, “housekeeping” protein), expressed approximately 10⁷ copies per cell and remained steady across different conditions (data not shown). Thus, the decreased levels of AAP, VP1, and VP2/3 are not likely caused by a decrease in cell numbers or health.

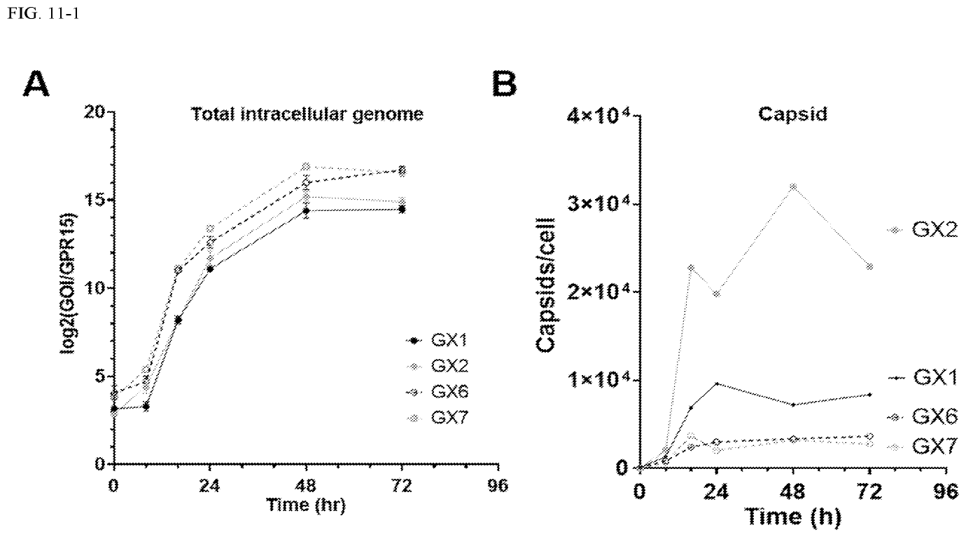

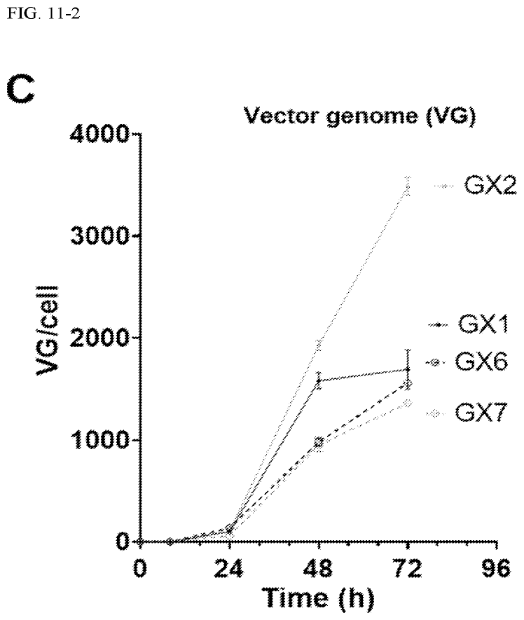

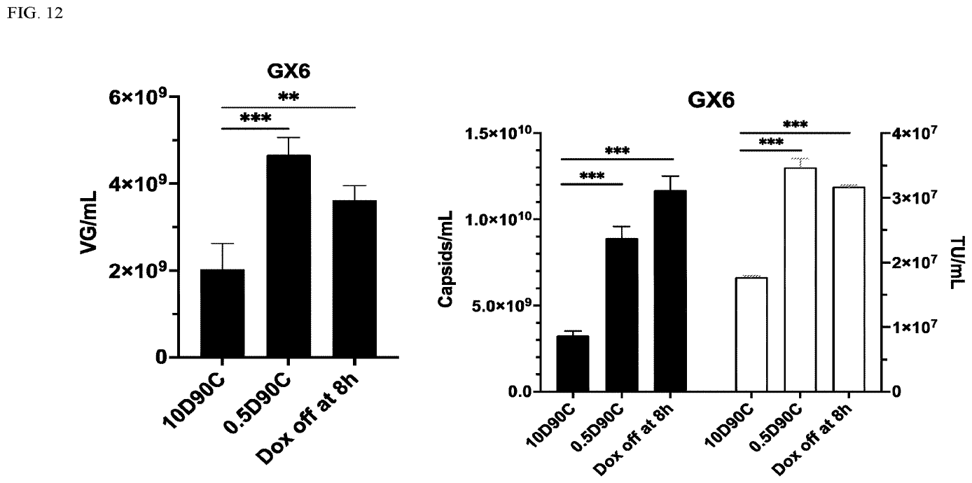

Total intracellular viral genome, including those encapsidated and free ones, reached ~10⁴ copies at 24 hours and increased to around 10⁵ copies by 48 hours (FIG. 11). From 1-2×10⁶ copies per cell of VP1/2/3 protein, about 10³-10⁴ were assembled into capsid at 24 hours (FIG. 11). Each capsid constitutes 60 VP subunits, hence around 20% to 50% of cap proteins were incorporated into capsids. The data also suggest that capsid assembly initiated early while VP1/2/3 level was still some way from its peak level around 24 hours. Compared with the dynamics of other viral components, genome packaging into capsid is a slow process (FIG. 11). Only a very small portion of total genomes (3%-0.5%) were encapsidated (Table 1). Genome encapsidation started early but the rate was slow until total genome copy reached about 10⁴-10⁵ copies per cell. In comparison, the molar ratio of VP1/2/3 proteins assembled into capsid to total VP1/2/3 proteins is higher. Thus, increasing synthesis of VP proteins may have more effect on increasing virus productivity than increasing the extent of genome amplification.

TABLE 1
Rates of genome encapsidation for select cell line clones.

| | | | | | | | | % VP1/2/3 |
|---|---|---|---|---|---|---|---|---|
| | | | | VP1/2/3 | DBP | Rep68 | % TG | protein |
| Cell | | | | protein | protein | protein | encapsidated | assembled |
| line | TG/cell | VG/cell | Capsid/cell | (copy/cell) | (copy/cell) | (copy/cell) | into VG | into capsid |
| GX1 | 6.90E+04 | 1693 | 8380 | 1.12E+06 | 1.42E+07 | 6.16E+05 | 2.5% | 45% |
| GX2 | 9.33E+04 | 3485 | 22900 | 2.72E+06 | 1.20E+07 | 1.54E+05 | 3.7% | 50% |
| GX6 | 3.23E+05 | 1561 | 3630 | 1.08E+06 | 2.49E+07 | 1.58E+06 | 0.5% | 20% |
| GX7 | 2.94E+05 | 1359 | 2750 | 7.75E+05 | 2.62E+07 | 3.22E+06 | 0.5% | 21% |

Method of Improving rAAV Production by Timed Induction

While constitutive, strong induction may yield high expression of target genes, these high levels of expression may not be optimal for rAAV production. To further improve production of rAAV, multiplexed screening of the concentration and time profile of doxycycline and cumate may be performed to identify ideal conditions for a particular cell line. Without wishing to be bound by theory, different cell lines may respond uniquely to promoter induction depending on the number and location of integrated polynucleotides. It may be most efficient to determine optimized induction parameters for an individual cell line by performing a multiplexed screening of induction parameters.

In one aspect, the present disclosure describes methods of increasing rAAV production in a cell line, the method including adding a predetermined concentration of an inducing molecule to a cell for a predetermined amount of time. The concentration of the inducing molecule may be determined, for example, by a multiplexed screening as described herein. The contact time of the inducing molecule may be determined, for example, by a multiplexed screening as described herein. In one or more embodiments, the molecule may be added to cell and later removed.

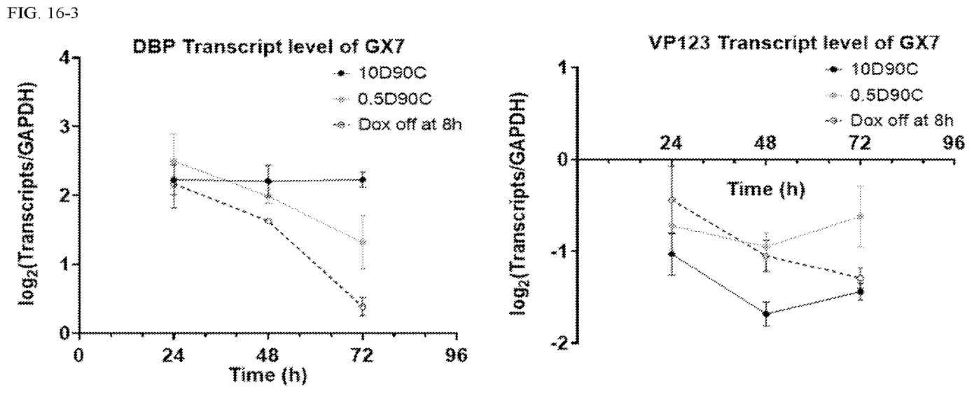

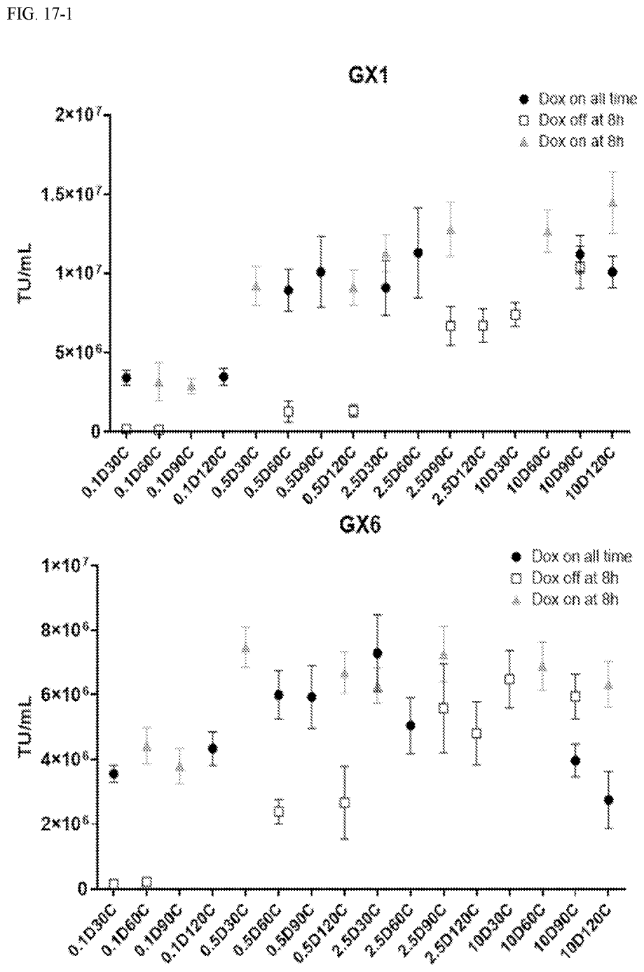

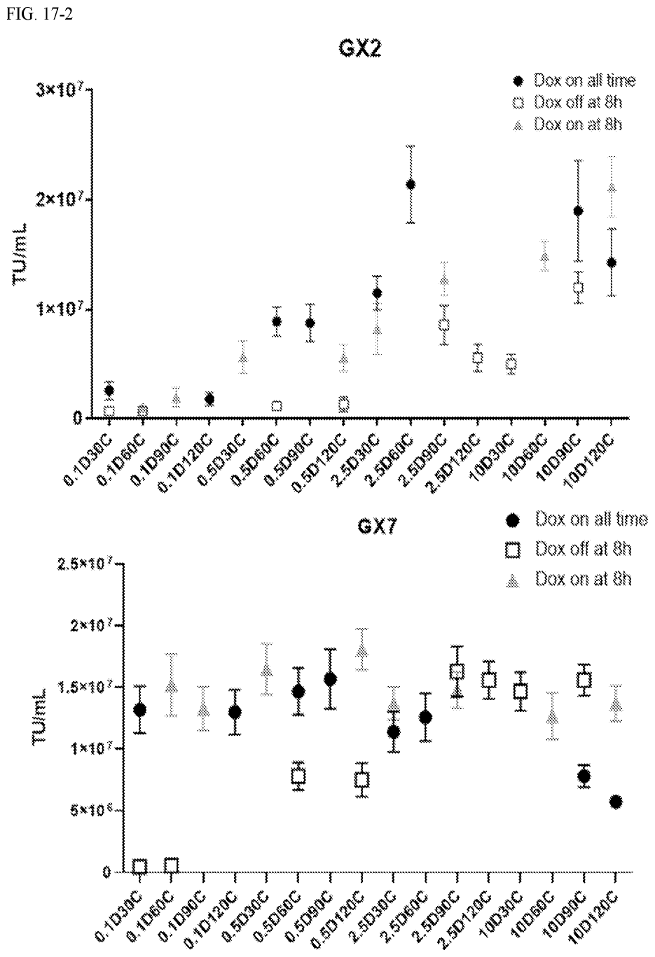

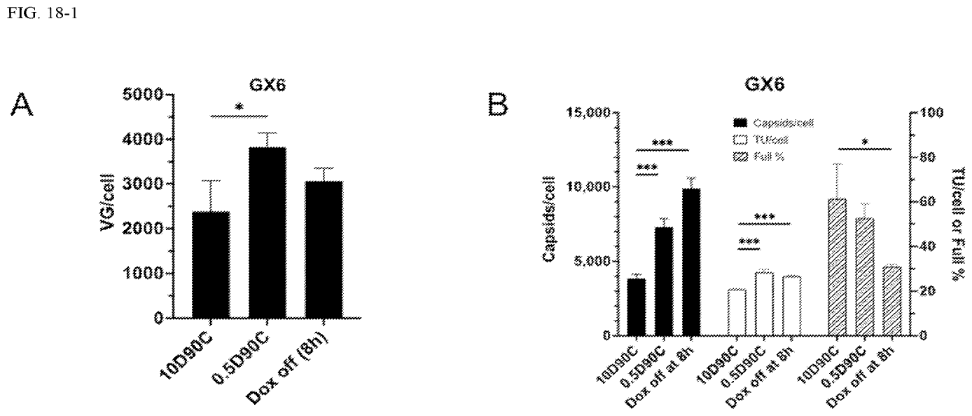

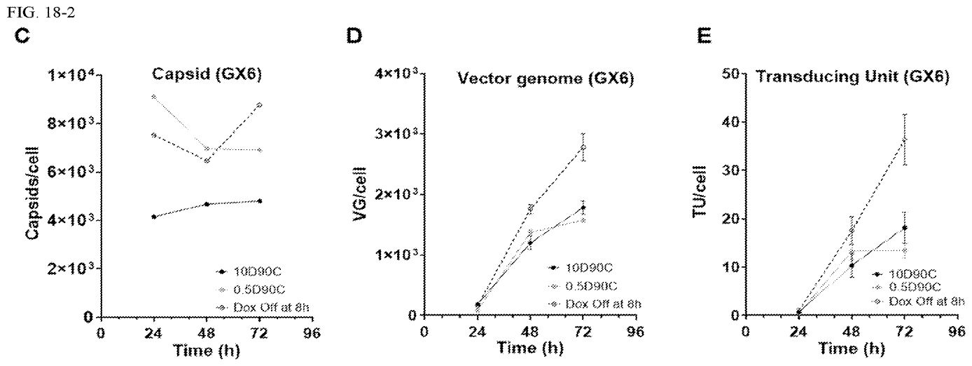

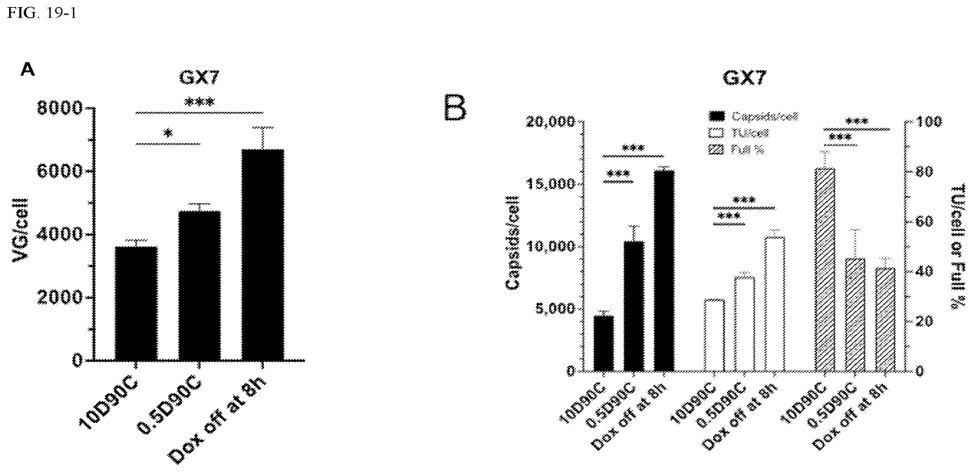

In one particular embodiment, multiplexed doxycycline/cumate induction screening was carried out for the four identified cell lines (FIG. 17). Using standard induction conditions (10D90C), GX1 and GX2 yielded an acceptably high titer of rAAV (FIG. 8). GX6 and GX7 had much higher titers by reducing the doxycycline level, removing doxycycline at 8 hours post-induction, or delaying addition of doxycycline. In view of this finding, new induction conditions were tested, reducing doxycycline from 10 μg/mL to 0.5 g/mL (0.5D90C) or removing the 10 μg/mL of doxycycline at 8 hpi (10D90C-8 h) to compare their titers to the original condition of 10D90C. The results confirmed that 0.5D90C and 10D90C-8 h gave higher VG, capsid, and TU titers on both a volumetric basis (FIG. 12, FIG. 14) and per cell basis (FIG. 18).

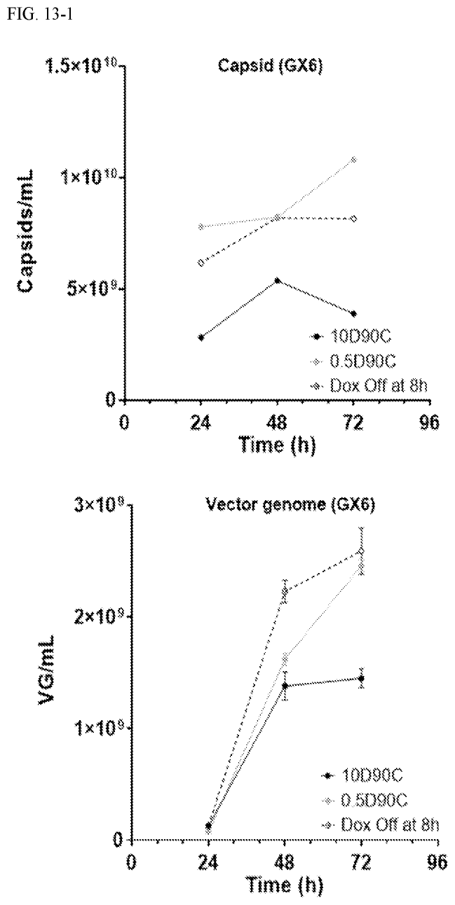

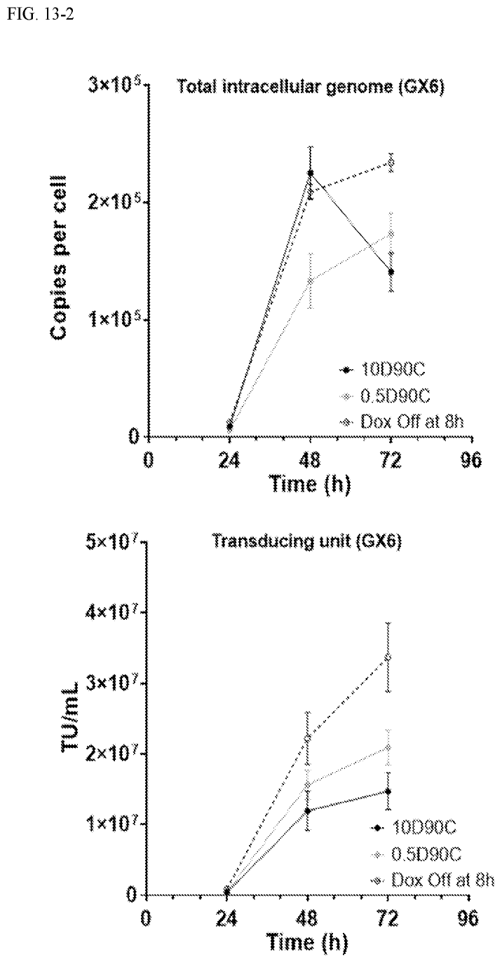

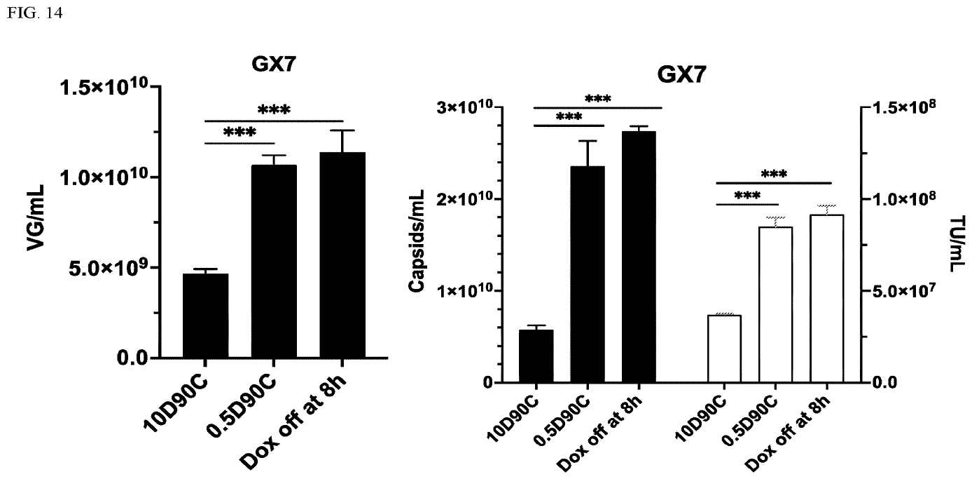

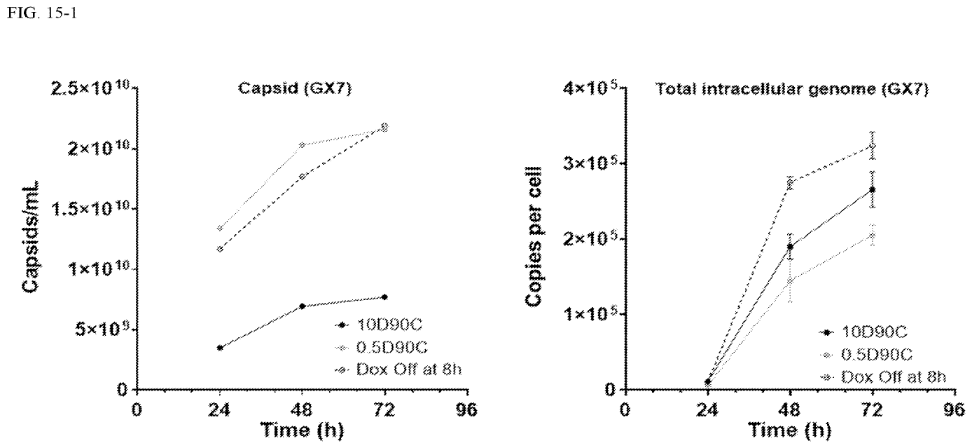

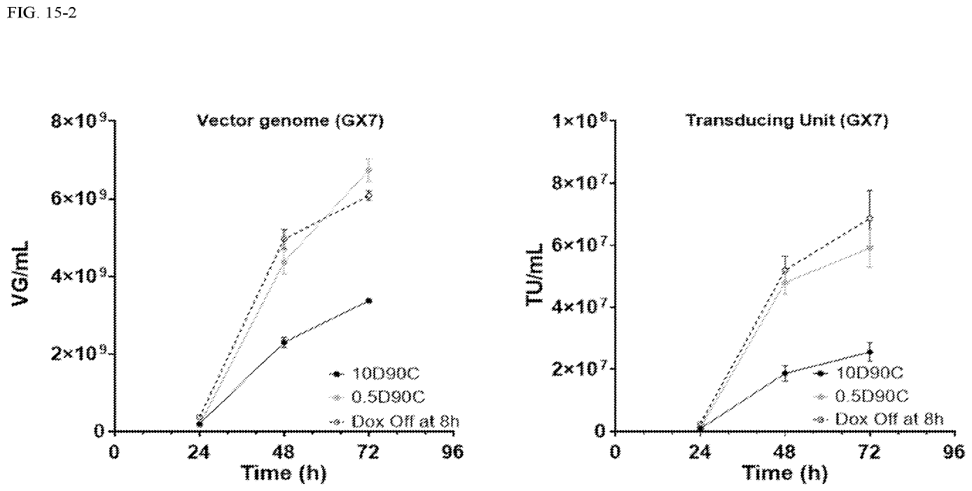

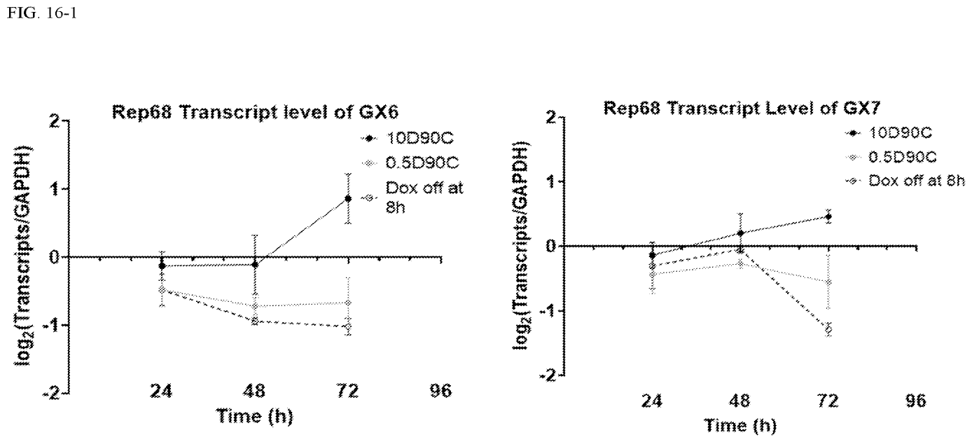

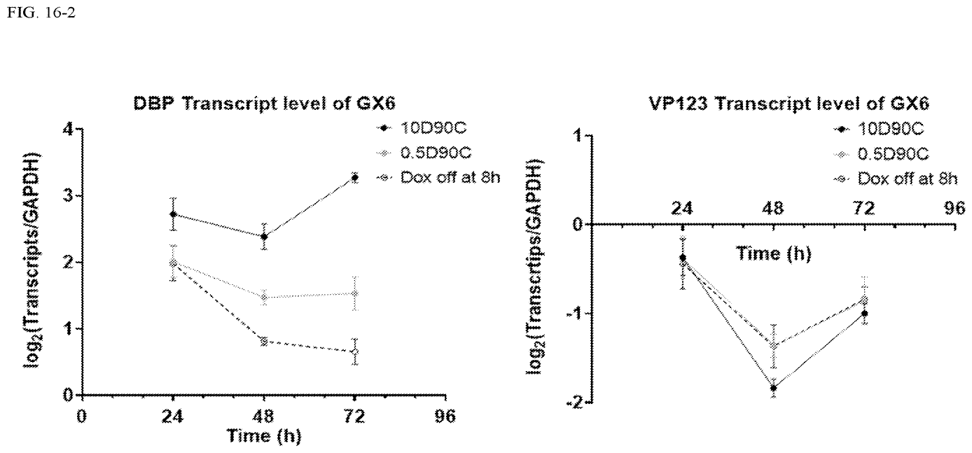

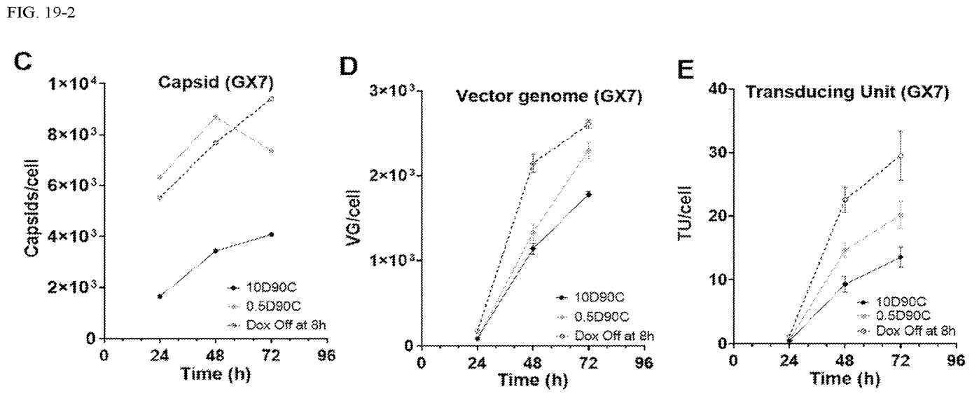

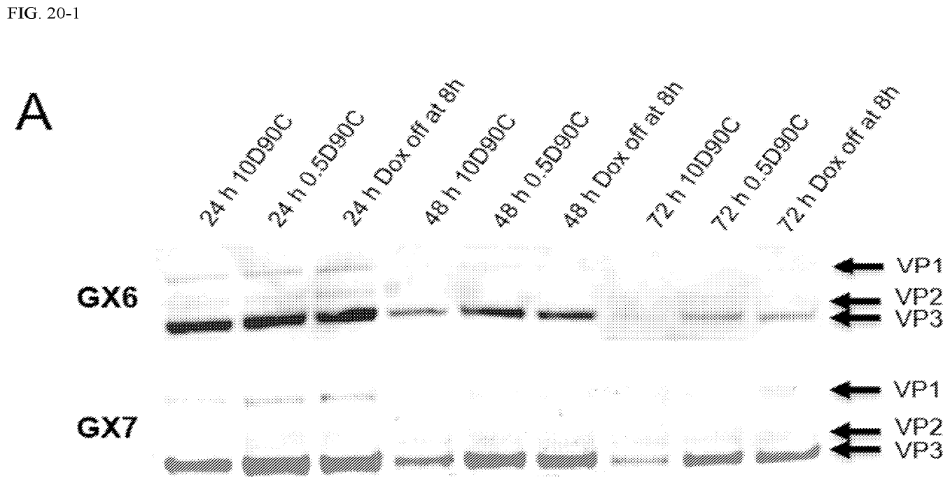

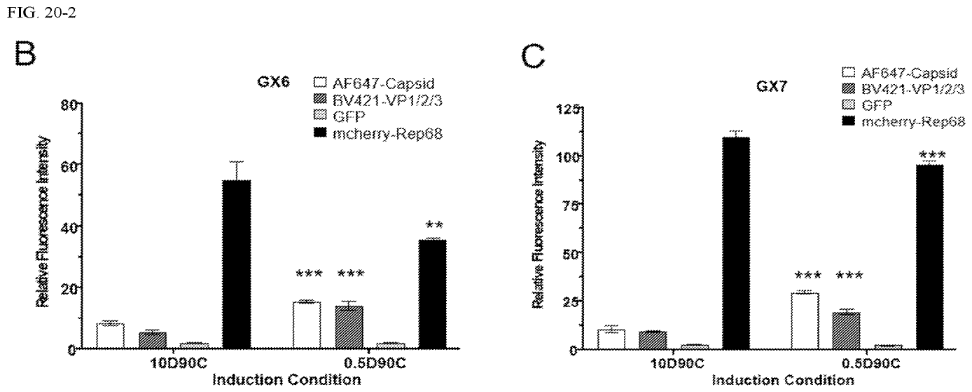

Further characterization was performed on the dynamics of viral gene products under these induction conditions using another set of cultures. Consistently, induction with 0.5D90C or 10D90C-8 h gave higher levels of VG, assembled capsids, and TU than induction with 10D90C. (FIG. 12, FIG. 13). However, the total intracellular genome was not markedly different among different induction conditions (FIG. 14, FIG. 15). At the transcript level, expression of Rep68 and DBP decreased significantly with a lower level or removal of doxycycline as expected (FIG. 16). Since the induction concentration of cumate was the same in all three conditions, VP123 transcript level was similar (FIG. 16). Nevertheless, the levels of assembled capsid were higher with lower level or removal of doxycycline (FIG. 13, FIG. 15), so was the level of VP1/2/3 proteins as measured by Western blots and flow cytometry (FIG. 20). This suggests that VP expression might be affected by post-transcriptional regulation.

The kinetic profiles of 0.5D90C and 10D90C-8 h were largely similar for both GX6 and GX7. Practically, removing doxycycline would require the replacement of medium and is difficult to implement in manufacturing. Accordingly, 0.5D90C was adopted for GX6 and GX7. These data demonstrate that each cell line showed optimal induction of gene expression in response to different induction protocols. This finding enables controlled expression of genes specific to each characterized cell line.

### Clone-Specific Optimal Induction

Using assay cell line RM4, rAAV productivity was surveyed at different combinations of inducer dose and time profile. Reduced induction of Tet driven promoter for Rep68 and DBP increased rAAV titer in GX6 and GX7. Large Rep proteins have been reported to inhibit translation and elicit apoptosis. Additionally, Rep78/68 was expressed actively early and then gave way to the expression of transcripts encoding Rep52/40 and VP1/2/3 proteins soon after infection, suggesting that low expression level of Rep68 might be preferred for optimal production. The beneficial effect of reduced doxycycline induction and Rep68 expression may reflect lessening negative effect of Rep68.

Method of Improving AAV Production by Adding One or more Small Molecules

In one aspect, the present disclosure describes a method of improving rAAV production including contacting an rAAV-producing cell with a small molecule. As is described herein, such as in Example 6, small molecule effectors of various cellular pathways may advantageously increase rAAV production.

In one or more embodiments, a method includes contacting an rAAV producing cell with a small molecule inhibitor of an interferon pathway. For example, a method may include contacting an rAAV producing cell with a small molecule inhibitor of the TANK binding kinase-1 (TBK1)/inhibitor of nuclear factor kappa-B kinase-epsilon (IKK-epsilon) complex. In one or more embodiments, a method includes contacting an rAAV producing cell with BX795 (N-[3-[[5-iodo-4-[3-(thiophene-2-carbonylamino)propylamino]pyrimidin-2-yl]amino]phenyl]pyrrolidine-1-carboxamide). In another example, a method may include contacting an rAAV producing cell with a small molecule inhibitor of the STING-mediated DNA signaling pathway. In one or more embodiments, a method includes contacting an rAAV producing cell with H151 STING. In another example, a method may include contacting an rAAV producing cell with a dsRNA sensor, such as dsRNA-dependent protein kinase R (PRK). In one or more embodiments, a method includes contacting an rAAV producing cell with PKR—IN—C16 (Compound 16, MedChemExpress LLC, Monmouth Junction, NJ).

In one or more embodiments, a method includes contacting an rAAV producing cell with a kinase inhibitor. For example, a method may include contacting an rAAV producing cell with PMA activated protein kinase C (PKC).

In one or more embodiments, a method includes contacting an rAAV producing cell with a small molecule that induces autophagy. A small molecule that induces autophagy may include, for example, a kinase inhibitor such as an AMP kinase (AMPK) inhibitor. For example, a method may include contacting an rAAV producing cell with dorsomorphin (sometimes referred to herein as “compound C”). A small molecule that induces autophagy may include, for example, a kinase activator, such as a protein kinase A (PKA) inhibitor. For example, a method may include contacting an rAAV producing cell with colforsin (also referred to as forskolin, coleonol, and HL 362).

A method may include contacting a cell with more than one small molecule, such as those described herein. Further, the small molecules described herein may be used in combination with the engineered cells and cell lines disclosed herein.

### Method of Improving AAV Production by Adding Protease Inhibitor

In one aspect, the present disclosure describes a method of improving rAAV production including contacting an rAAV-producing cell with a proteasome inhibitor. In one or more embodiments, a method includes contacting a cell line of the present disclosure with a proteasome inhibitor. It should be noted that a “proteasome inhibitor” is distinct from a “protease inhibitor.” Different proteasome inhibitors had meaningfully different effects on rAAV production. In one or more embodiments, the proteasome inhibitor may be MG132.

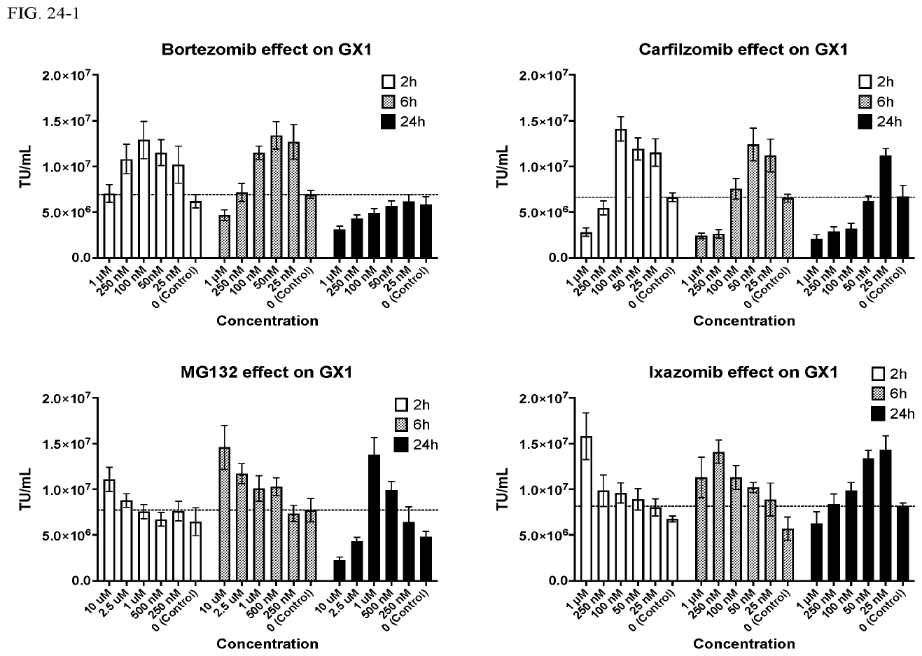

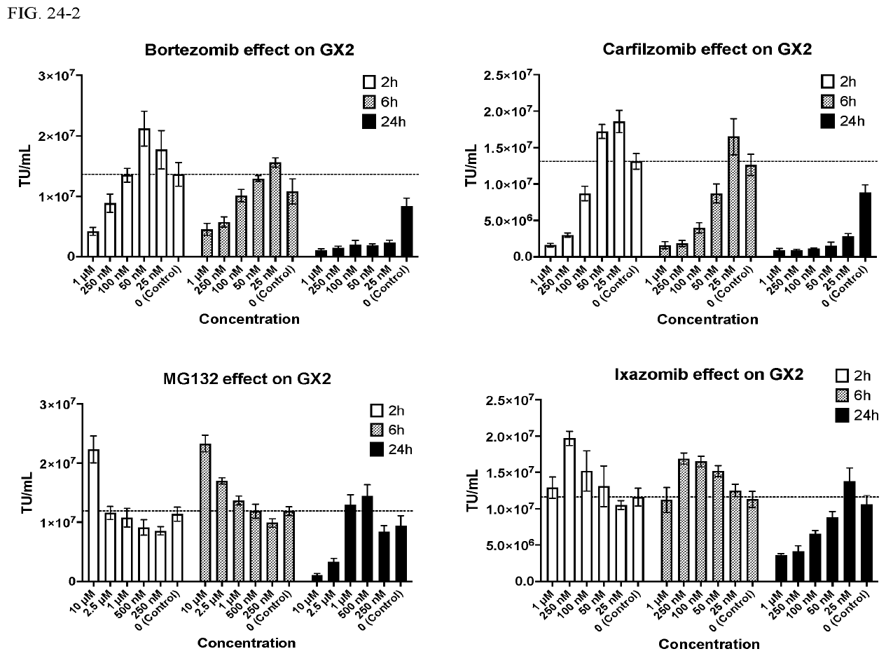

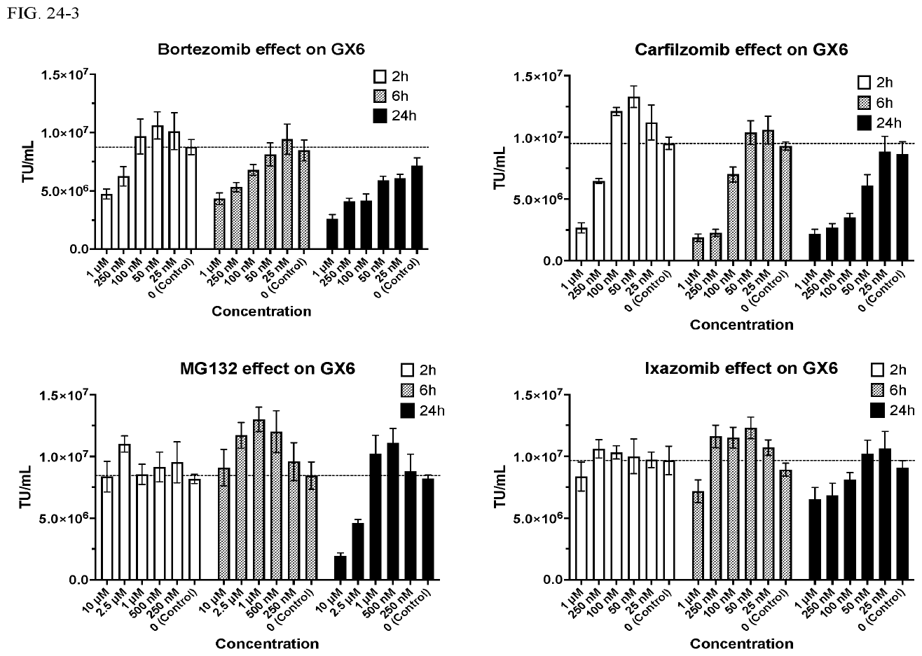

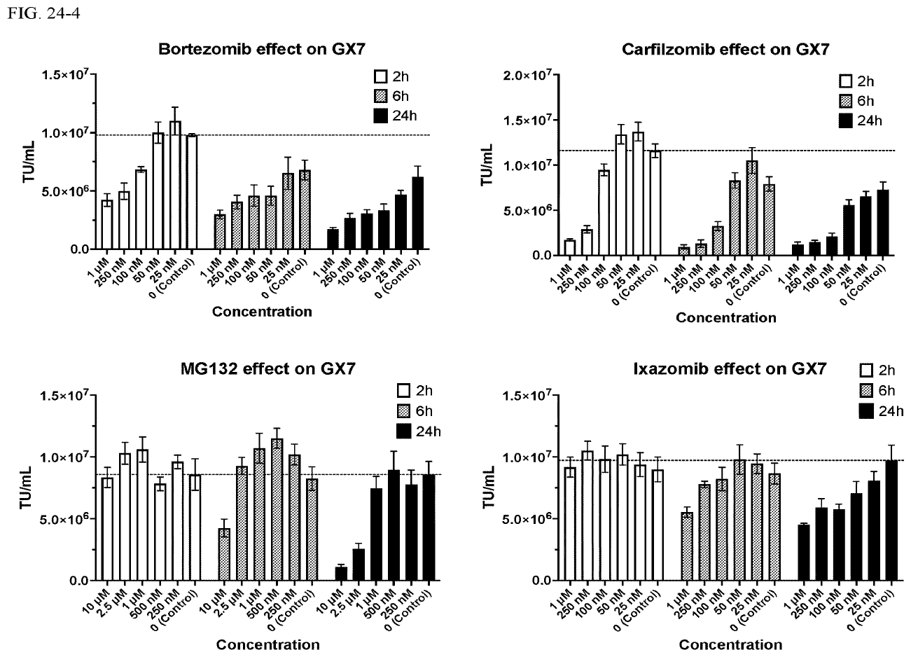

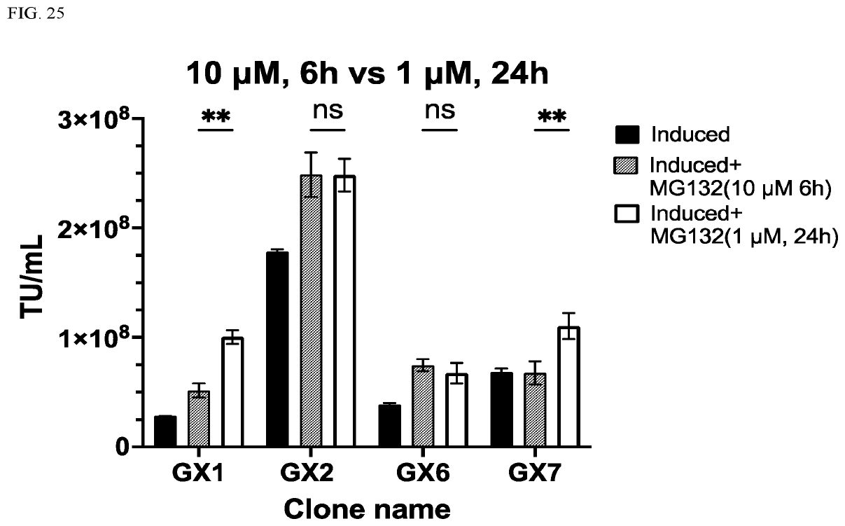

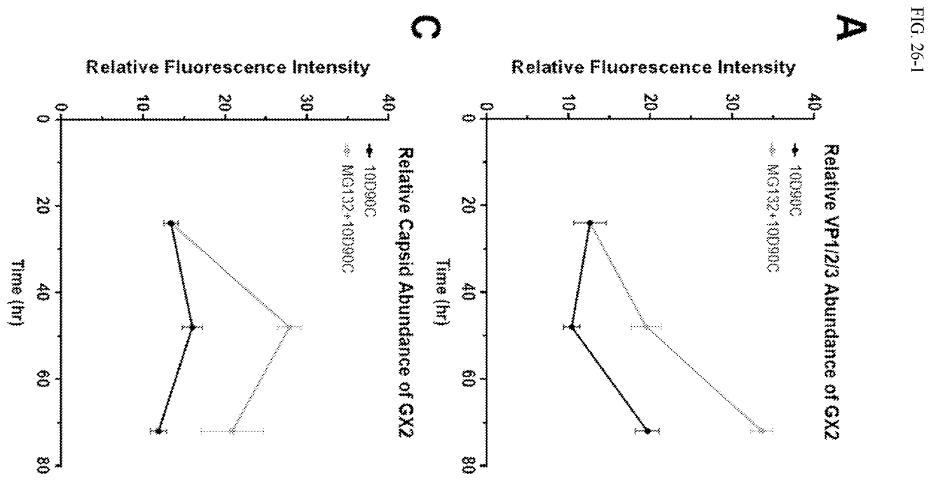

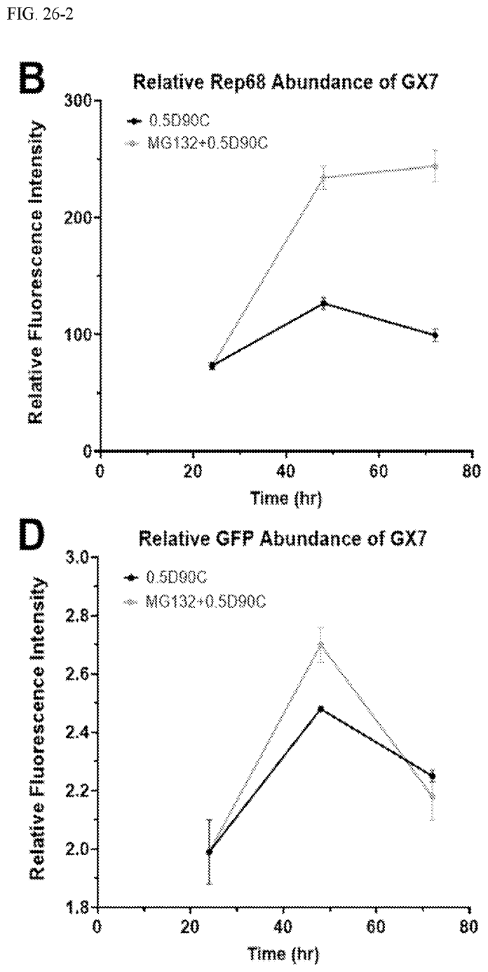

An observation made regarding the dynamic behavior of viral proteins through targeted proteomics was the decline of VP1/2/3 and AAP after reaching peak around 24 hours (FIG. 10). The decrease may have been caused by proteolytic degradation. Thus, four proteome inhibitors were screened for their effect on the infectious rAAV titer under different conditions on the four GX cell lines. An inhibitor was added to cultures at 24 hpi under optimal conditions and incubated for two hours, six hours, or 24 hours before being removed for the remainder of the 72 hours. Among the four inhibitors tested, MG132 had a consistent enhancing effect on TU titer on all four cell lines tested (FIG. 24). Two conditions, MG132 (10 μM, 6 hours) and MG132 (1 μM, 24 hours) were further evaluated and both showed enhancing effect on the rAAV productivity as compared to the case of without MG132. For GX2 and GX6, both conditions had a similar enhancing effect, but for GX1 and GX7 a higher titer was obtained with MG132 (1 μM, 24 hours) (FIG. 25). In subsequent studies, the MG132 at 1 M with incubation for 24 hours were used for all cell lines.

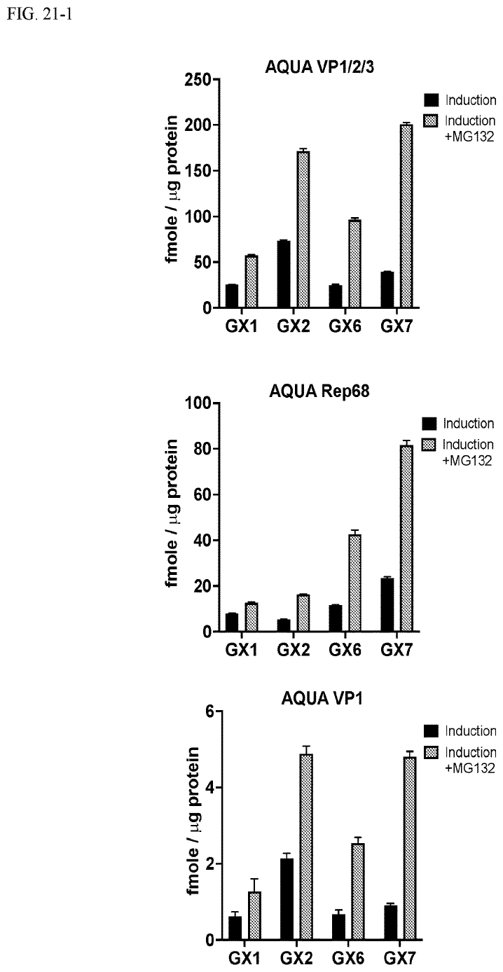

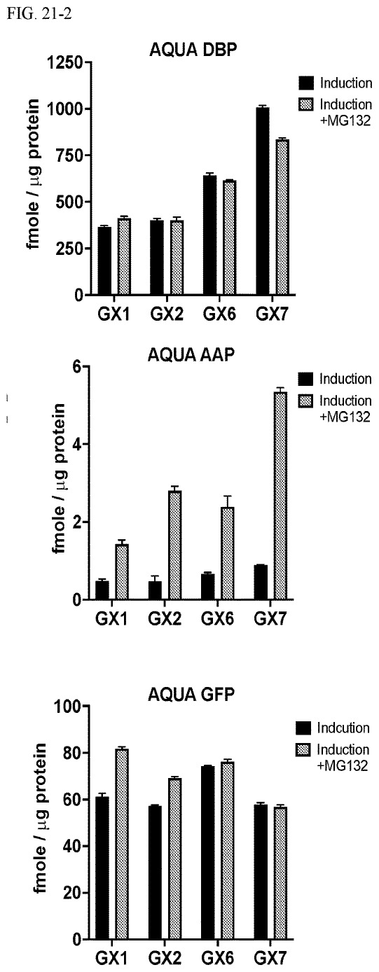

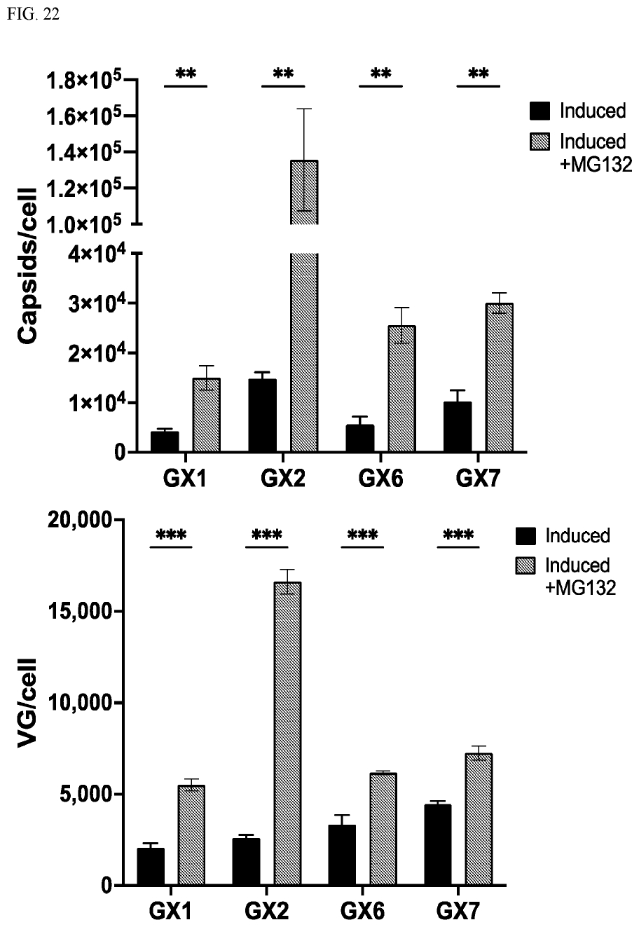

Targeted proteomics quantification was performed on the cell samples of induction only and induction with MG132 (1 μM, 24 hours). VP1/2/3, VP1 and AAP protein levels were significantly increased for all four cell lines when treated with MG132 (FIG. 21). Since Rep68 was tagged with a destabilization domain, Rep68 abundance also increased by inhibition of proteasomal degradation (FIG. 21). In comparison, the abundance levels of viral protein DBP and viral genome encoded GFP did not increase upon inhibition of proteosome (FIG. 21). The effect on viral protein abundance was further verified by immunofluorescence intensity measured by flow cytometry (FIG. 21, FIG. 22). With optimal induction condition and the treatment of MG132, GX2 clones can produced more than 10⁴ VG and over 10′ capsids per cell (FIG. 22).

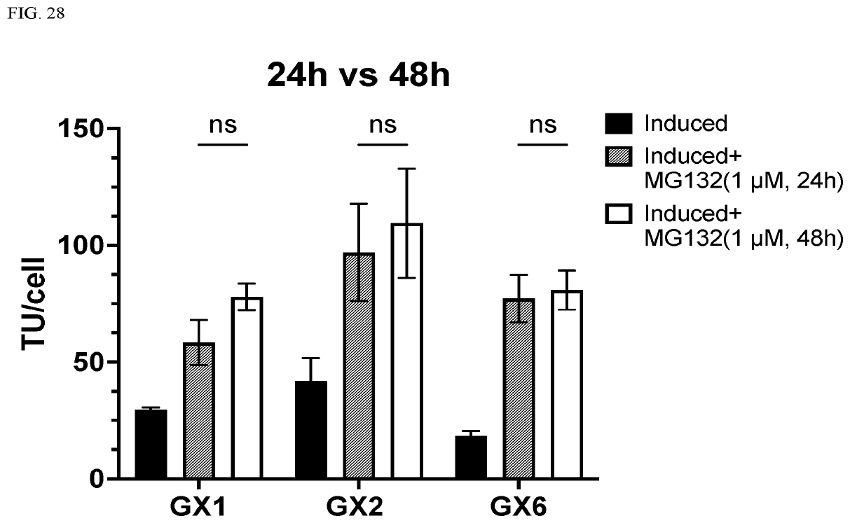

This targeted proteomic study revealed possible degradation of AAP and VP1/2/3 proteins after reaching peak value in all four GX cell lines (FIG. 10). AAP facilitates cotransport of VP proteins to the nucleoli and promotes VP protein stability and capsid assembly by oligomerization. Without AAP, VP proteins are subject to degradation in several pathways including proteasomal degradation in unfolded protein response (UPR) which is frequently incurred in host cell response to virus infection in general and has been reported to occur in rAAV transduction. Adding proteasome inhibitor in rAAV transduction has been shown to have an enhancing effect on transgene expression. The effect of inhibiting proteasome-mediated degradation after induction of GX cell lines was explored and revealed increased AAV2 capsid proteins and rAAV production. The enhancing effect of rAAV productivity was attainable using a low MG132 concentration of 1 μM throughout the induction (i.e., without removing MG132 at 48 hpi) (FIG. 28).

While MG132 treatment diminishes VP and AAP degradation and boosted capsid titer, the rate of encapsidation did not increase proportionally, suggesting that encapsidation is likely rate limiting for full particle packaging. The full particle content ranged from 24%-81% depending on cell lines and induction conditions. The value was comparable to or higher than the 30% cited in literature.

VG:TU ratio is often used as an estimate of rAAV infectivity. This value can be highly variable between production methods or even preparation lots. rAAV produced from GX cells across different batches of production had VG:TU about 100, a value comparable to prior reports.

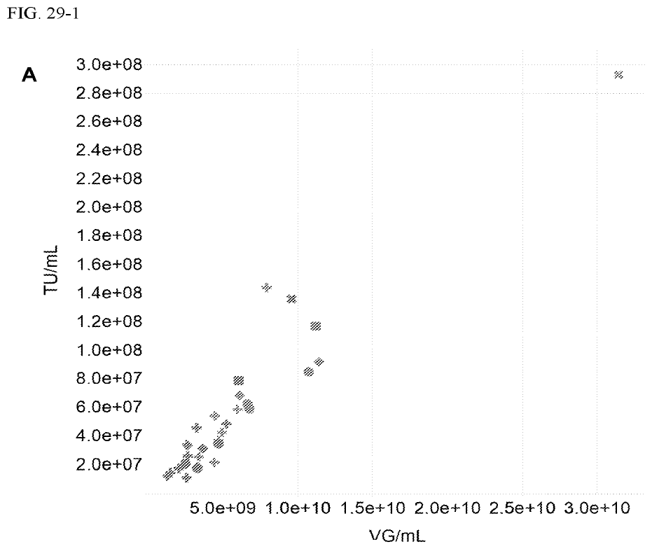

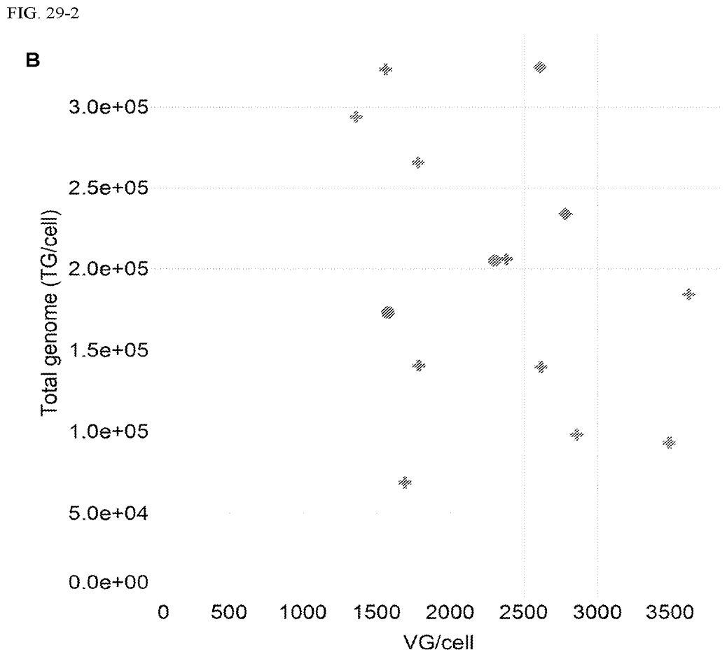

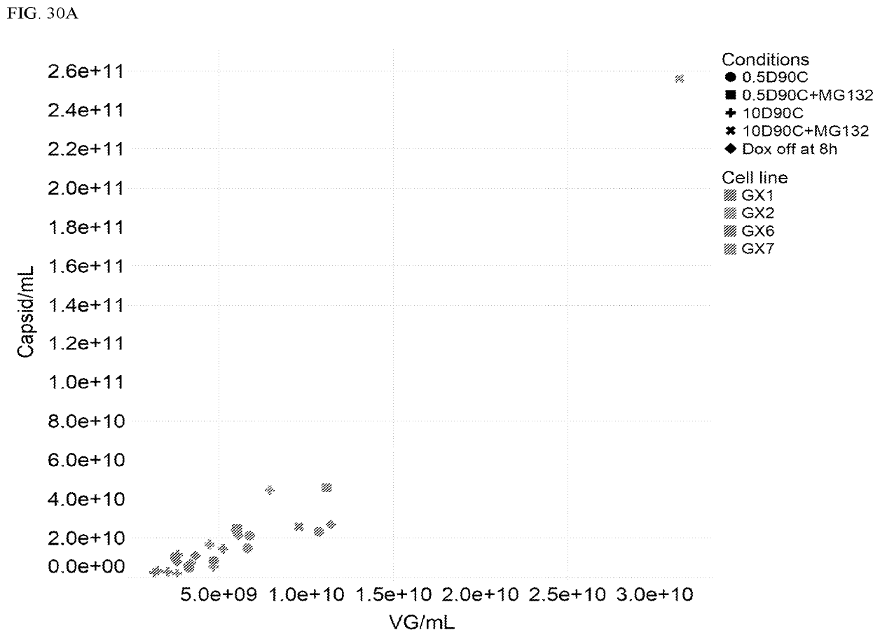

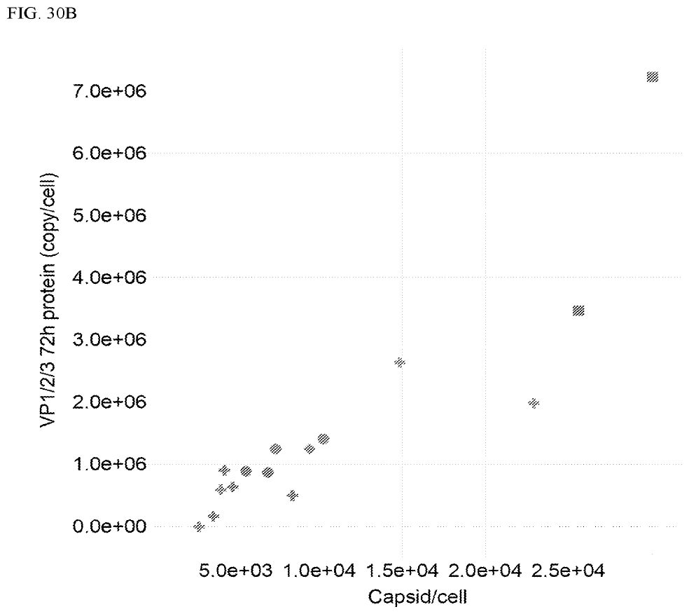

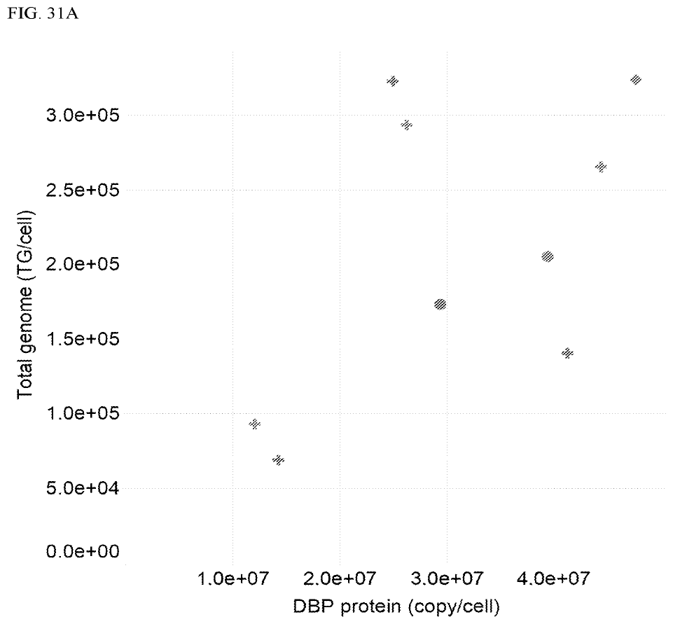

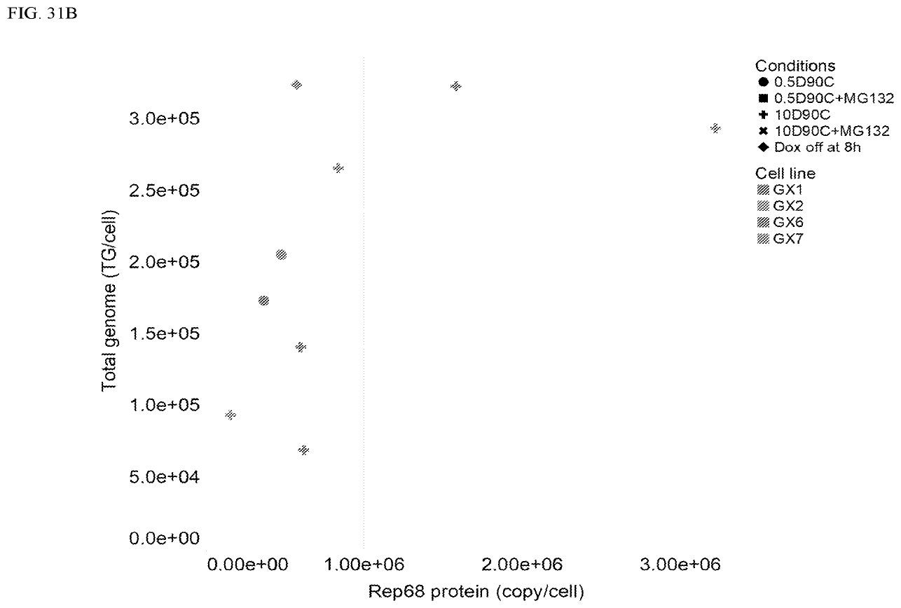

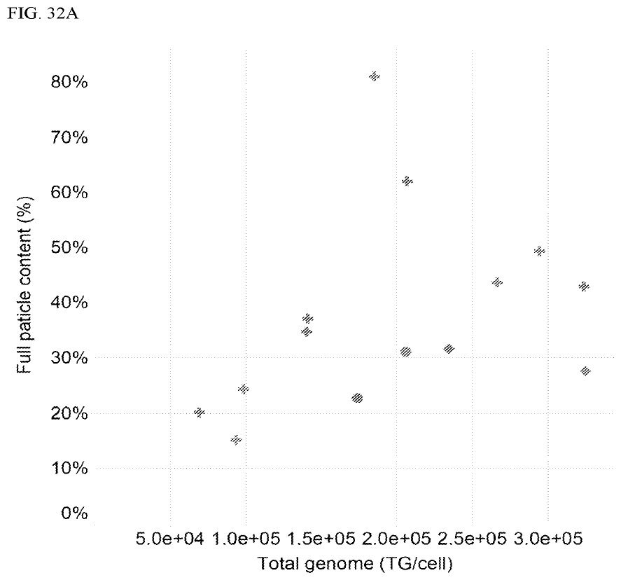

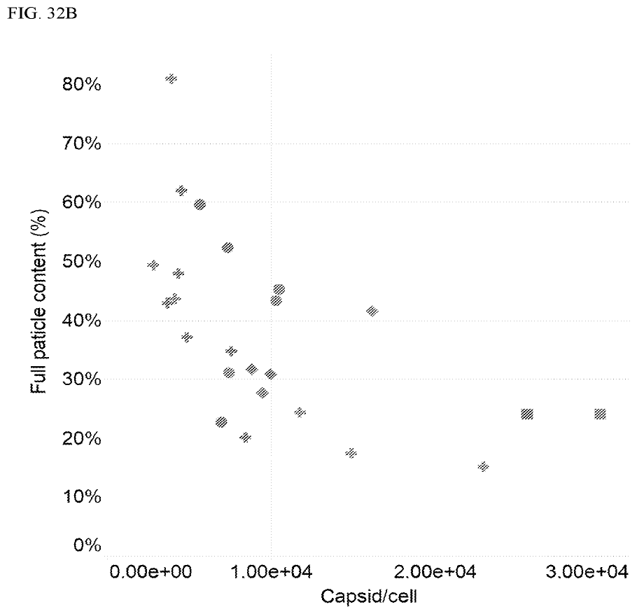

This disclosure reports success in further enhancing rAAV productivity through cell engineering and optimization of gene expression dynamics and cultural conditions. The productivity of the GX cell lines approaches that of commonly used triple plasmid transient transfection. Correlation between viral components and VG titers suggests that the productivity can possibly be enhanced by the higher expression of cap genes and increasing the efficiency of viral genome packaging into capsids (FIGS. 29-32). Further exploration of helper viral components such as Ad non-coding VA RNA which inhibits PKR-mediated antiviral response and other Ad E4 proteins (e.g., E4orf3, E4orf6/7) which modulate host responses along with tempering of host cell response to rAAV replication may have much enhancing effect on the productivity.

### Method of Increasing Full Particle Content

In one or more embodiments, a cell line or method of the present disclosure may produce rAAV having an increased number of full capsids relative to a comparable rAAV produced using conventional methods of rAAV production. The content of full rAAV capsids produced by an rAAV preparation may be expressed as a ratio of full to empty capsids or as a percentage of full capsids (relative to the total number of full and empty capsids). For example, a method described herein may produce an rAAV preparation having at least 40%, at least 45%, at least 50%, at least 55%, at least 60%, at least 65%, or at least 70% full capsids. A method described herein may produce an rAAV preparation having a ratio of 1:2, 1:1, 1.5:1.2:1, or 3:1 empty to full capsids.

The number of full capsids in a given preparation may be measured by electron microscopy (EM), such as cryo-EM or transmission EM. Alternatively or additionally, the number of full capsids in a given preparation may be measured by calculating the total number of assembled capsids using, for example, an ELISA assay, measuring the total number of protected genomes using, for example, RT-qPCR, and calculating the ratio of genomes to assembled capsids. In theory, a preparation of 100% full capsids would be determined to have the same number of assembled capsids and protected genomes.

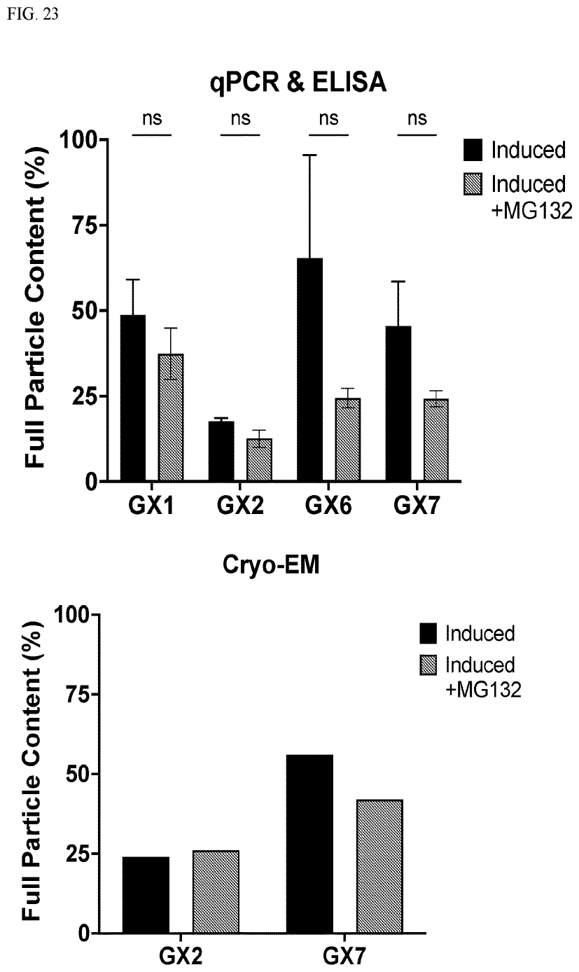

Using all four GX cell lines, the full particle content produced under optimal induction conditions with or without MG132 was evaluated. The content determined using the number of VG and capsids showed that while total viral particles increased with MG132 treatment, the full particle content was insignificantly lower (FIG. 23). Thus, while MG132 increased assembled capsid, the encapsidation rate did not proportionately increase. Full particle content values calculated using RT-qPCR and ELISA assays. In addition, negative-staining transmission electron microscopy (TEM) and Cryo-EM were carried out to obtain a more precise count of empty and full rAAV particles.

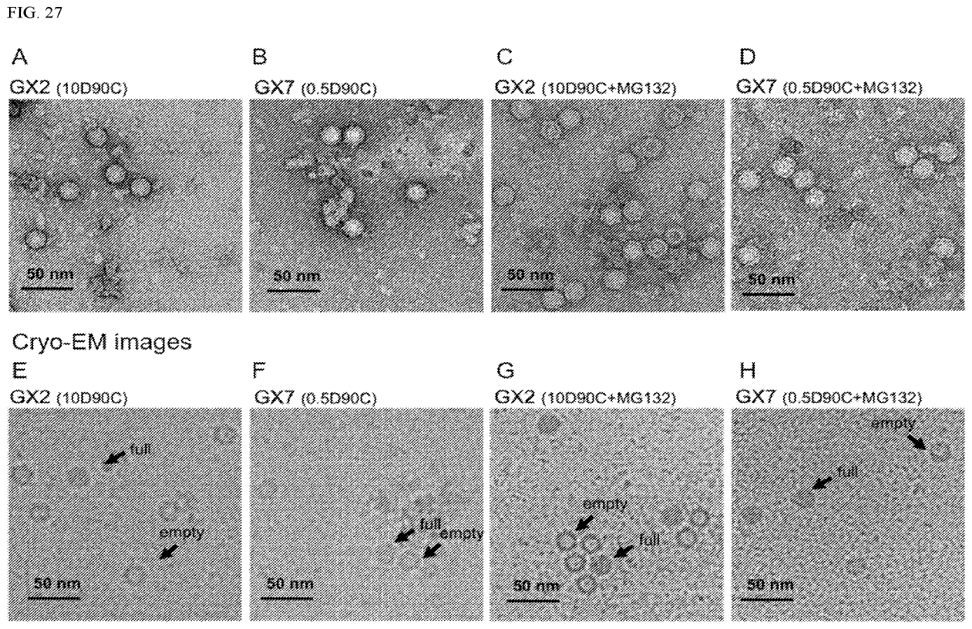

rAAV produced from two cell lines, GX2 and GX7, were prepared for EM imaging and analysis (FIG. 27). The full particle contents were 24% and 56% without MG132 and remained at a similar level (26%) and dropped moderately (42%) with MG132 treatment for GX2 and GX7 respectively (FIG. 23). Although slightly lower full particle contents, which were still comparable to or better than triple transfection, were seen in high producers treated with MG132, highly increased amount of VG compensated for lower full particle contents.

In addition, Example 3 of the present disclosure describes that varying the dynamics of expression of proteins associated with replication and of capsid proteins can significantly increase the percentage of full capsids in a preparation of rAAV. In particular, delaying expression of capsid proteins until after replication-associated proteins. In one or more embodiments, a method includes modulating expression of capsid proteins. In one or more embodiments, a method includes delaying expression of capsid proteins until expression of other proteins associated with viral replication. For example, in embodiments wherein the capsid proteins are under control of an inducible promoter, the method may include adding the inducer after other proteins associated with viral replication are expressed in the cell. In one or more embodiments where the capsid proteins are under control of a first inducible promoter and replication proteins are under control of a second inducible promoter, the method includes contacting the cell with the first inducer before contacting the cell with the second inducer.

The present Examples describe that inducing expression of the capsid proteins at least 16 hours after inducing expression of the replication proteins. However, desirable expression timing may vary depending on multiple factors, such as the capsid protein used, the size of the genome, and the type of producer cell used.

In one or more embodiments, a method includes expressing the capsid proteins at least 4, at least 5, at least 6, at least 7, at least 8, at least 9, at least 10, at least 11, at least 12, at least 13, at least 14, at least 15, at least 16, at least 17, at least 18, at least 19, or at least 20 hours after expressing the replication proteins. In one or more embodiments wherein expression of the capsid proteins is controlled by an inducible promoter, the method includes contacting the cell with the corresponding inducer when the cell is expressing replication proteins. In one or more embodiments, the cell constitutively expresses the replication proteins. In one or more embodiments, the replication proteins are under control of an inducible promoter. Typically, the replication proteins are under control of a different inducible promoter than the capsid proteins. Thus, in one or more embodiments, the method includes contacting the cell with a second inducer, wherein the second inducer induces expression of one or more of the replication proteins, and subsequently contacting the cell with a first inducer, wherein the first inducer induces expression of the capsid proteins.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one, especially in combination with the term “comprising”; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Reference throughout this specification to “one embodiment,” “an embodiment,” “certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

As used herein, the word “exemplary” means to serve as an illustrative example and should not be construed as preferred or advantageous over other embodiments.

The words “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

In several places throughout the above description, guidance is provided through lists of examples, which examples can be used in various combinations. In each instance, the recited list serves only as a representative group and should not be interpreted as an exclusive list.

For any method disclosed herein that includes discrete steps, the steps may be performed in any feasible order. And, as appropriate, any combination of two or more steps may be performed simultaneously.

### EXEMPLARY EMBODIMENTS

Embodiment 1 is a stable mammalian cell line including: a first polynucleotide encoding a first AAV inverted terminal repeat (ITR) sequence and a first promoter, wherein the first promoter is not expressed in the stable mammalian cell line.

Embodiment 2 is the stable mammalian cell line of Embodiment 1, wherein the first promoter is a tissue-specific promoter.

Embodiment 3 is the stable mammalian cell line of Embodiment 1, wherein the first promoter is a first inducible promoter.

Embodiment 4 is the stable mammalian cell line of Embodiment 2, further including a second polynucleotide encoding a repressor sequence wherein the first inducible promoter is operably linked to the repressor.

Embodiment 5 is the stable mammalian cell line of any preceding Embodiment, wherein the first polynucleotide further includes a second ITR sequence.

Embodiment 6 is the stable mammalian cell line of Embodiment 5, wherein the first promoter is located between the first ITR sequence and the second ITR sequence.

Embodiment 7 is the stable mammalian cell line of any preceding Embodiment, wherein the first polynucleotide encodes a gene of interest (GOI).

Embodiment 8 is the stable mammalian cell line of Embodiment 7, wherein the GOI encodes a protein.

Embodiment 9 is the stable mammalian cell line of Embodiment 7, wherein the GOI is operably linked to the first promoter.

Embodiment 10 is the stable mammalian cell line of any preceding Embodiment, wherein the first polynucleotide further includes a selectable marker.

Embodiment 11 is the stable mammalian cell line of any one of Embodiments 4-10, wherein the repressor blocks expression of the first inducible promoter.

Embodiment 12 is the stable mammalian cell line of any one of Embodiments 4-11, wherein the inducible promoter is active in the absence of the repressor.

Embodiment 13 is the stable mammalian cell line of any preceding Embodiment, further including: a third polynucleotide encoding an AAV large replicate (Rep) protein, wherein the third polynucleotide is operably linked to a second inducible promoter; a fourth polynucleotide encoding an adenovirus (Ad) E4orf6, wherein the fourth polynucleotide is operably linked to a third inducible promoter; and a fifth polynucleotide encoding an Ad DNA binding protein (DBP), wherein the fifth polynucleotide is operably linked to a fourth inducible promoter.

Embodiment 14 is the stable mammalian cell line of Embodiment 13, wherein the second inducible promoter, the third inducible promoter, and the fourth inducible promoter have at least 80% sequence identity.

Embodiment 15 is the stable mammalian cell line of Embodiment 13, wherein the second inducible promoter, the third inducible promoter, and the fourth inducible promoter are induced by a common stimulus.

Embodiment 16 is the stable mammalian cell line of Embodiment 13 wherein: the first promoter includes a lactose-inducible promoter, a doxycycline-inducible promoter, a mifepristone-inducible promoter, or a cumate-inducible promoter; the second inducible promoter includes a lactose-inducible promoter, a doxycycline-inducible promoter, a mifepristone-inducible promoter, or a cumate-inducible promoter; the third inducible promoter includes a lactose-inducible promoter, a doxycycline-inducible promoter, a mifepristone-inducible promoter, or a cumate-inducible promoter; and a fourth inducible promoter includes a lactose-inducible promoter, a doxycycline-inducible promoter, a mifepristone-inducible promoter, or a cumate-inducible promoter.

Embodiment 17 is the stable mammalian cell line of Embodiment 13 wherein: the first promoter includes a lactose-inducible promoter; the second inducible promoter includes a doxycycline-inducible promoter; the third inducible promoter includes a doxycycline-inducible promoter; and a fourth inducible promoter includes a doxycycline-inducible promoter.

Embodiment 18 is the stable mammalian cell line of any one of Embodiments 13-17, wherein the third polynucleotide is tagged with a destabilization domain.

Embodiment 19 is the stable mammalian cell line of Embodiment 18, wherein the destabilization domain is ligand-responsive.

Embodiment 20 is the stable mammalian cell line of Embodiment 19, wherein the destabilization domain is a mutant FKBP12 destabilization domain responsive to Shield-1.

Embodiment 21 is the stable mammalian cell line of any of Embodiments 13-20, further including: a sixth polynucleotide encoding an AAV capsid protein, wherein the sixth polynucleotide is operably linked to a fifth inducible promoter; and a seventh polynucleotide encoding an AAV small Rep protein, wherein the seventh polynucleotide is operably linked to a sixth inducible promoter.

Embodiment 22 is the stable mammalian cell line of Embodiment 21 wherein: the fifth inducible promoter includes a lactose-inducible promoter, a doxycycline-inducible promoter, a mifepristone-inducible promoter, or a cumate-inducible promoter; and a sixth inducible promoter includes a lactose-inducible promoter, a doxycycline-inducible promoter, a mifepristone-inducible promoter, or a cumate-inducible promoter.

Embodiment 23 is the stable mammalian cell line of Embodiment 21 wherein: the fifth inducible promoter includes a cumate-inducible promoter; and the sixth inducible promoter includes a cumate-inducible promoter.

Embodiment 24 is the stable mammalian cell line of any of Embodiments 21-23, including at least two copies of the first polynucleotide and two copies of the second polynucleotide for each copy of the third polynucleotide.

Embodiment 25 is a stable mammalian cell line, including: a first polynucleotide encoding an AAV large replicate (Rep) protein, wherein the first polynucleotide is operably linked to a first inducible promoter; a second polynucleotide encoding an adenovirus (Ad) E4orf6, wherein the second polynucleotide is operably linked to a second inducible promoter; a third polynucleotide encoding an Ad DNA binding protein (DBP), wherein the third polynucleotide is operably linked to a third inducible promoter; a fourth polynucleotide encoding an AAV capsid protein, wherein the fourth polynucleotide is operably linked to a fourth inducible promoter; and a fifth polynucleotide encoding an AAV small Rep protein, wherein the fifth polynucleotide is operably linked to a fifth inducible promoter.

Embodiment 26 is the stable mammalian cell line of Embodiment 25, further including a sixth polynucleotide encoding a first AAV inverted terminal repeat (ITR) sequence.

Embodiment 27 is the stable mammalian cell line of Embodiment 26, wherein the sixth polynucleotide lacks a promoter.

Embodiment 28 is the stable mammalian cell line of Embodiment 26, wherein the sixth polynucleotide includes a sixth inducible promoter.

Embodiment 29 is the stable mammalian cell line of Embodiment 26, wherein the sixth polynucleotide includes a tissue-specific promoter that is not expressed in the stable mammalian cell line.

Embodiment 30 is the stable mammalian cell line of Embodiment 25, wherein the first inducible promoter, the second inducible promoter, and the third inducible promoter have at least 80% sequence identity.

Embodiment 31 is the stable mammalian cell line of Embodiment 25, wherein the first inducible promoter, the second inducible promoter, and the third inducible promoter are induced by a common stimulus.

Embodiment 32 is the stable mammalian cell line of Embodiment 31, wherein the common stimulus includes doxycycline.

Embodiment 33 is the stable mammalian cell line of any one of Embodiments 25-32, wherein the first polynucleotide is tagged with a destabilization domain.

Embodiment 34 is the stable mammalian cell line of Embodiment 25 wherein: the fourth inducible promoter includes a cumate-inducible promoter; and the fifth inducible promoter includes a cumate-inducible promoter.

Embodiment 35 is the stable mammalian cell line of any of Embodiments 25-34, including at least two copies of the fourth polynucleotide and two copies of the fifth polynucleotide for each copy of the first polynucleotide.

Embodiment 36 is the stable mammalian cell line of any preceding Embodiment, wherein at least one promoter is an exogenous promoter.

Embodiment 37 is the stable mammalian cell line of any preceding Embodiment, wherein at least one promoter is a non-AAV promoter.

Embodiment 38 is the stable mammalian cell line of any preceding Embodiment, wherein at least one inducible promoter includes a doxycycline-inducible promoter, a mifepristone-inducible promoter, a lactose-inducible promoter, or a cumate-inducible promoter.

Embodiment 39 is the stable mammalian cell line of any preceding Embodiment, further including an exogenous DNA polymerase.

Embodiment 40 is the stable mammalian cell line of Embodiment 39, wherein the exogenous DNA polymerase is a DNA-dependent DNA polymerase.

Embodiment 41 is the stable mammalian cell line of Embodiment 39, wherein the exogenous DNA polymerase is a viral DNA polymerase.

Embodiment 42 is the stable mammalian cell line of Embodiment 41, wherein the viral DNA polymerase is HSV-1 DNA polymerase.

Embodiment 43 is the stable mammalian cell line of any one of Embodiments 39-42, wherein the stable mammalian cell line includes a polynucleotide encoding the exogenous DNA polymerase.

Embodiment 44 is a method of using the stable mammalian cell line of any one of Embodiments 1 to 43.

Embodiment 45 is the method of Embodiment 44, wherein: the stable mammalian cell line includes the cell line of any one of Embodiments 3-43, and the method includes contacting the mammalian cell line with an inducer of one or more of the inducible promoters.

Embodiment 46 is the method of Embodiment 45, further including measuring a concentration of infectious recombinant AAV particles.

Embodiment 47 is the method of Embodiment 46, wherein the concentration of infectious recombinant AAV particles is measured by fluorescence or immunofluorescence analysis of the stable mammalian cell line.

Embodiment 48 is the method of Embodiment 46, wherein the concentration of infectious recombinant AAV particles is measured by quantitative polymerase chain reaction (PCR) analysis of the stable mammalian cell line.

Embodiment 49 is the method of Embodiment 45, wherein the percentage of full rAAV particles is measured.

Embodiment 50 is the method of Embodiment 49, wherein the percentage of full rAAV particles is measured using electron microscopy.

Embodiment 51 is the method of Embodiment 49, wherein the percentage of full rAAV particles is at least 35%.

Embodiment 52 is the method of any one of Embodiments 44-51, wherein the method includes contacting the cell with a predetermined amount of inducer, wherein the predetermined amount of inducer is selected to increase the percentage of full rAAV particles.

Embodiment 53 is the method of any one of Embodiments 44-52, further including generating an AAV particle.

Embodiment 54 is the method of one of Embodiments 44-53, wherein the inducer includes lactose, IPTG, doxycycline, tetracycline, mifepristone, or cumate.

Embodiment 55 is the method of any one of Embodiments 44-54, further including contacting the cell line with a proteasome inhibitor.

Embodiment 56 is the method of Embodiment 55, wherein the proteasome inhibitor is MG132.

Embodiment 57 is the method of any one of Embodiments 44-56, further including removing the inducer from the cell line.

Embodiment 58 is the method of any one of Embodiments 44-57, wherein the method includes contacting the cell line with more than one inducer.

Embodiment 59 is a stable mammalian cell line including: a third polynucleotide encoding an AAV large replicate (Rep) protein, wherein the third polynucleotide is operably linked to a second inducible promoter; a fourth polynucleotide encoding an adenovirus (Ad) E4orf6, wherein the fourth polynucleotide is operably linked to a third inducible promoter; a fifth polynucleotide encoding an Ad DNA binding protein (DBP), wherein the fifth polynucleotide is operably linked to the fourth inducible promoter, wherein the second inducible promoter and the third inducible promoter have at least 80% sequence identity; a sixth polynucleotide encoding an AAV capsid protein, wherein the sixth polynucleotide is operably linked to a fifth inducible promoter; and a seventh polynucleotide encoding an AAV small Rep protein, wherein the seventh polynucleotide is operably linked to a sixth inducible promoter.

Embodiment 60 is a method of using the stable mammalian cell line of Embodiment 59, the method including transfecting the cell line with: a first polynucleotide encoding a first AAV inverted terminal repeat (ITR) sequence and a first inducible promoter; and a second polynucleotide encoding a repressor sequence wherein the first inducible promoter is operably linked to the repressor.

Embodiment 61 is a method of using the stable mammalian cell line of Embodiment 59, the method including infecting the cell line with a vector encoding: a first polynucleotide encoding a first AAV inverted terminal repeat (ITR) sequence and a first inducible promoter; and a second polynucleotide encoding a repressor sequence wherein the first inducible promoter is operably linked to the repressor.

Embodiment 62 is a stable mammalian cell line including: a second polynucleotide encoding a repressor sequence; a third polynucleotide encoding an AAV large replicate (Rep) protein, wherein the third polynucleotide is operably linked to a second inducible promoter; a fourth polynucleotide encoding an adenovirus (Ad) E4orf6, wherein the fourth polynucleotide is operably linked to a third inducible promoter; a fifth polynucleotide encoding an Ad DNA binding protein (DBP), wherein the fifth polynucleotide is operably linked to the third inducible promoter, wherein the second inducible promoter and the third inducible promoter have at least 80% sequence identity; a sixth polynucleotide encoding an AAV capsid protein, wherein the sixth polynucleotide is operably linked to a fifth inducible promoter; and a seventh polynucleotide encoding an AAV small Rep protein, wherein the seventh polynucleotide is operably linked to a sixth inducible promoter.

Embodiment 63 is a method of using the stable mammalian cell line of Embodiment 62, the method including transfecting the cell line with: a first polynucleotide encoding a first AAV inverted terminal repeat (ITR) sequence and a first inducible promoter, wherein the first inducible promoter is operably linked to the repressor.

Embodiment 64 is a method of using the stable mammalian cell line of Embodiment 62, the method including infecting the cell line with a vector encoding:
a first polynucleotide encoding a first AAV inverted terminal repeat (ITR) sequence and a first inducible promoter, wherein the first inducible promoter is operably linked to the repressor.

Embodiment 65 is a method of using the stable mammalian cell line of any one of Embodiments 1 to 43, wherein the method includes transfecting the cell line with one or more polynucleotides of Embodiments 1 to 25.

Embodiment 66 is a method of using the stable mammalian cell line of any one of Embodiments 11 to 43, wherein the method includes: (i) contacting the cell with a second inducer, wherein the second inducer induces expression of the second inducible promoter, contacting the cell with a third inducer, wherein the third inducer induces expression of the third inducible promoter, or a fourth inducer, wherein the fourth inducer induces expression of the fourth inducible promoter; (ii) contacting the cell with a first inducer, wherein the first inducer induces expression of the first inducible promoter.

Embodiment 67 is the method of Embodiment 66, wherein the method includes contacting the cell with the second inducer, the third inducer, and the fourth inducer.

Embodiment 68 is the method of Embodiment 66 or 67, wherein the cell is contacted with the first inducer at least 10 hours after the cell is contacted with the second inducer, the third inducer, and/or the fourth inducer.

### EXAMPLES

The present invention is illustrated by the following examples. It is to be understood that the particular examples, materials, amounts, and procedures are to be interpreted broadly in accordance with the scope and spirit of the invention as set forth herein.

### Example 1

### Vectors

The rAAV2-GFP genome from pAAV-CAG-GFP (Addgene, Watertown, MA) was cloned into a Leap-In transposon backbone (ATUM) with a lacI repressor gene linked to a puromycin resistance gene driven by a phosphoglycerate kinase promoter, and CAG promoter was replaced with RSV-LacO promoter (Agilent Technologies, Inc., Santa Clara, CA), yielding the inducible rAAV genome module (GM, FIG. 1). The construction of the replication module (RM) and packaging module (PM) was previously described. Modification of RM was made by replacing the GENESWITCH (Thermo Fisher Scientific, Inc., Waltham, MA) promoter with a TetON promoter and removing GeneSwitch transactivator gene, yielding the new RM (FIG. 1). pAAV-CAG-GFP (Addgene, Watertown, MA), pAAV-RC2 (Cell Biolabs, Inc., San Diego, CA), and pHelper vectors (Cell Biolabs, Inc., San Diego, CA) were used for triple transfection.

Generation of rAAV Producer Cell Lines

HEK293 cells (Cell Biolabs, Inc., San Diego, CA) were cultured in Dulbecco's Modified Eagle Medium (DMEM) supplemented with 10% fetal bovine serum and 1× antibiotic-antimycotic. GX stable cell lines were created by transposon-mediated integration (ATUM) with three genetic modules followed by selection, single-cell cloning, and productivity screening. A schematic of this workflow is shown in FIG. 6.

Cell Culture and rAAV Vector Production

For rAAV production by GX cell lines, cells were seeded at 3.75×10⁵ cells per well in 6-well plates. At 24 hpi, media was replaced with induction media containing 10 μg/mL of doxycycline and 90 g/mL of cumate (Sigma-Aldrich, St. Louis, MO) unless noted otherwise. For proteasome inhibitor experiments, 1 μM of MG132 (InvivoGen, San Diego, CA) was added to GX cells at 24 hpi, and cells were incubated with inhibitors for 24 hours before media replacement unless noted otherwise. Cells were harvested at 72 hpi. Extraction methods and qPCR assays of intracellular DNA and RNA were previously described.

rAAV Preparation and Characterization

Preparation of benzonase-treated rAAV crude lysate and titration of encapsidation vector genome (VG) by qPCR, capsids by ELISA and transducing units by RM4 assay cells were previously described. The full particle content was obtained by dividing the VG by the capsid titer.

### ELISA Capsid Quantification

The AAV2 and AAV8 capsid titers were measured using AAV2 and AAV8 titration ELISA kits (Progen, Inc., Wayne, PA). Manufacturer's instructions were followed.

### Western Blot

Clarified cell lysate were quantified by BCA protein assay (Thermo Fisher Scientific, Inc., Waltham, MA). 10 μg of protein was loaded to each lane of NuPAGE Bis-Tris gels (Thermo Fisher Scientific, Inc., Waltham, MA), transferred membranes were incubated with mouse anti-AAV2 VP1/2/3 (Progen, Inc., Wayne, PA) and anti-beta-actin (Cell Signaling Technology, Inc., Danvers, MA) monoclonal antibodies (mAbs), and then incubated with anti-mouse IgG conjugated with alkaline phosphatase mAb. Membranes were incubated with IMMUN-STAR AP substrate and enhancer (Bio-Rad Laboratories, Inc., Hercules, CA) for five minutes before chemiluminescence detection.

### Flow Cytometry Assay

Cells were fixed with 4% paraformaldehyde, permeabilized with 0.2% Triton X-100, incubated with mouse anti-AAV2 VP1/2/3 (Progen, Inc., Wayne, PA) and mouse anti-AAV2 intact particle (Progen, Inc., Wayne, PA) mAbs, then incubated with rat anti-mouse IgG1 mAb conjugated with BV421 fluorescence (BioLegend, San Diego, CA) and rat anti-mouse IgG3 mAb conjugated with ALEXA FLUOR 647 fluorescence (Abcam, Cambridge, United Kingdom). Then cells were analyzed on a flow cytometer (LSR II, Becton, Dickenson and Co., Franklin Lakes, NJ) for GFP, mCherry, BV421, and Alexa Fluor 647 fluorescence.

### Targeted Quantitative Proteomics

Sample processing, heavy isotope-labeled peptides, parameters used for data acquisition by mass spectrometry with parallel reaction monitoring method, and data analysis were described previously.

### Transmission Electron Microscopy and Cryo-EM

rAAV crude lysate was produced in multiple 150 mm dishes, then purified using affinity chromatography (POROS CAPTURESELECT AAVX, Thermo Fisher Scientific, Inc., Waltham, MA). For negative-staining TEM, 5 μl of the purified AAV sample was applied to a glow-discharged 400 mesh ultra-thin carbon grid (Electron Microscopy Sciences, Hatfield, PA) and stained by 2% uranyl formate solution (w/v), then blotted dry. For cryo-EM, 3 μl of sample was applied to a glow-discharged R2/1 200 mesh grids (QUANTIFOIL TEM, Ted Pella, Inc., Redding, CA). The blotted dry grid was plunged into liquid ethane, then transferred at the liquid N2 temperature to an 300 kV field emission gun TEM (FEI Tecnai, Thermo Fisher Scientific, Inc., Waltham, MA). The images were recorded on a slow scan CCD camera (Gatan 4k, Gatan, Inc., Pleasanton, CA) or a direct electron detector camera (SUMMIT K2, Gatan, Inc., Pleasanton, CA).

### Sequential Transfection

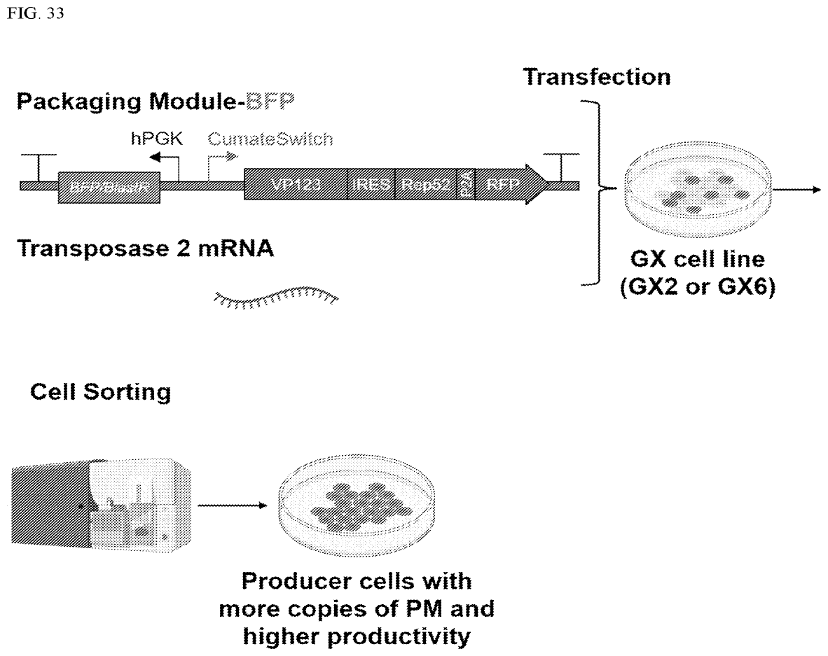

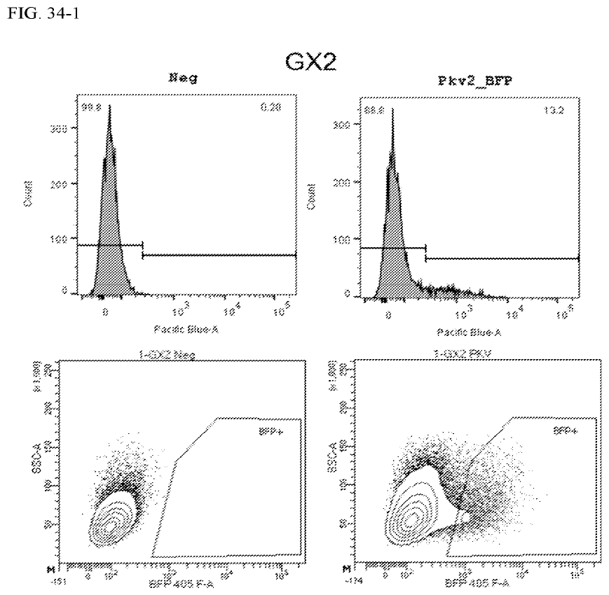

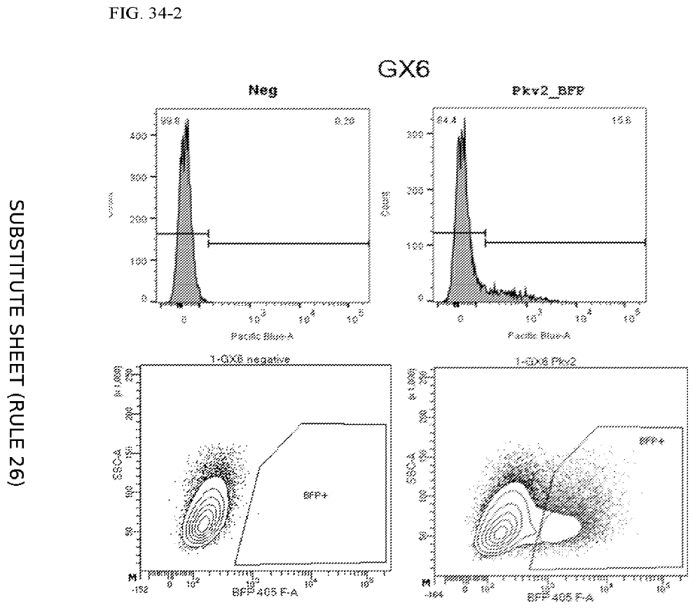

In one example, two selected clones (GX2, GX6) that had previously integrated the PM, RM, and GM described above were subsequently transfected with an additional PM polynucleotide. This second PM polynucleotide included a BFP/blasticidin resistance cassette. After transfection, cells were sorted for expression of BFP. It was thought that the cells that were positive for BFP would have increased expression of the PM. Flow cytometry plots showing selection of the BFP-positive clones are shown in FIG. 34.

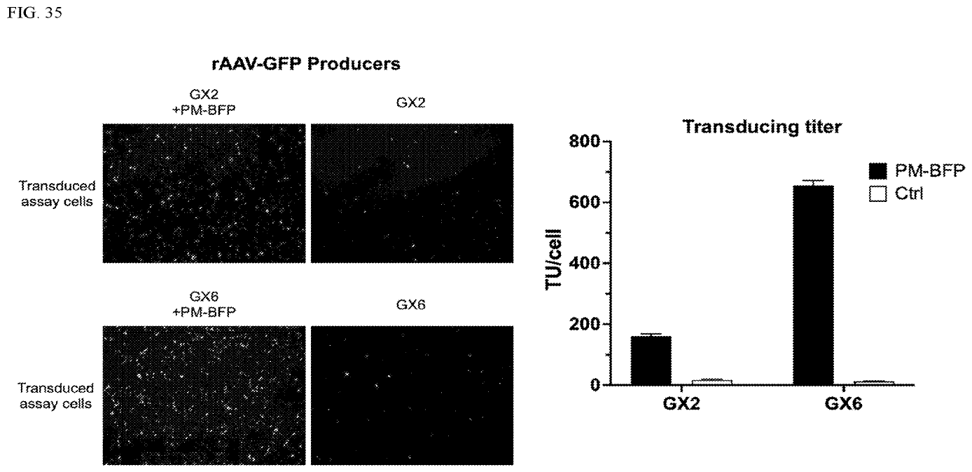

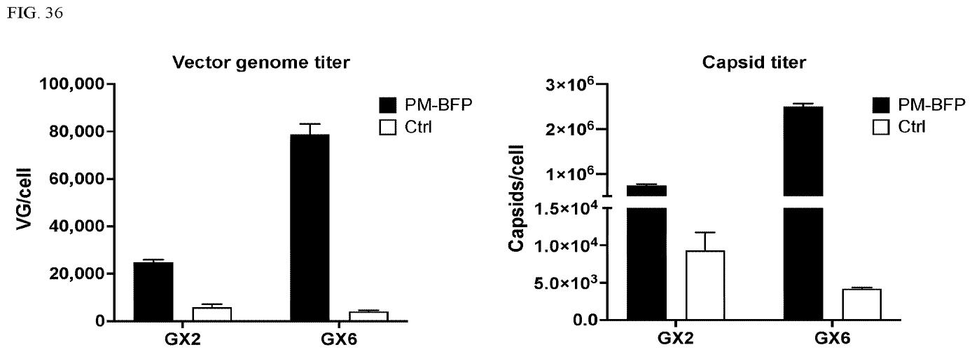

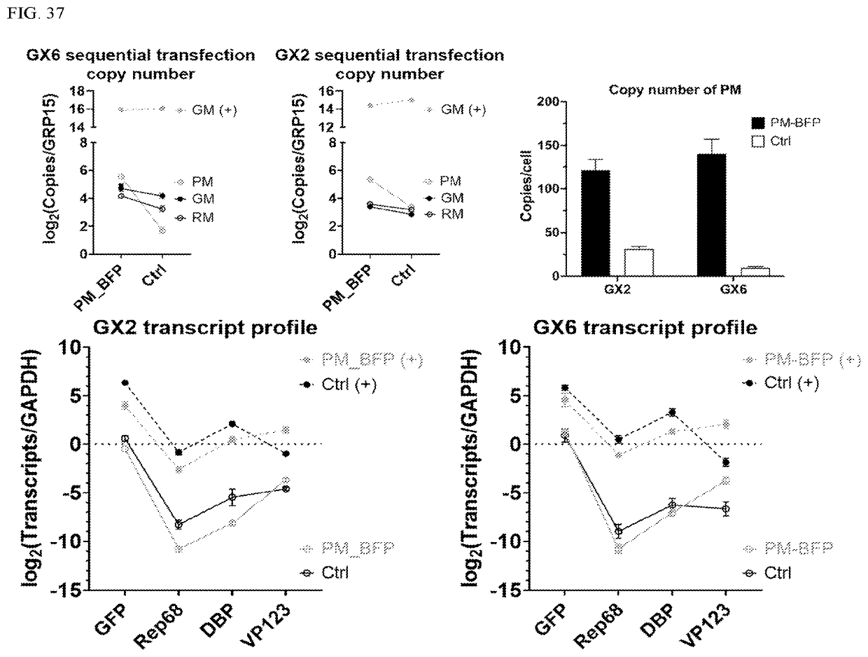

These BFP-positive cells were used for production of rAAV as described herein. Significantly higher titers were achieved as compared to the clones before integration of the second PM polynucleotide. Fluorescence of transduced cells as well as quantification of the rAAV produced are shown in FIG. 35 and FIG. 36. Significantly higher integrated copies of PM and induced VP123 transcript level were achieved as compared to the clones before integration of the second PM polynucleotide (FIG. 37).

### Example 2

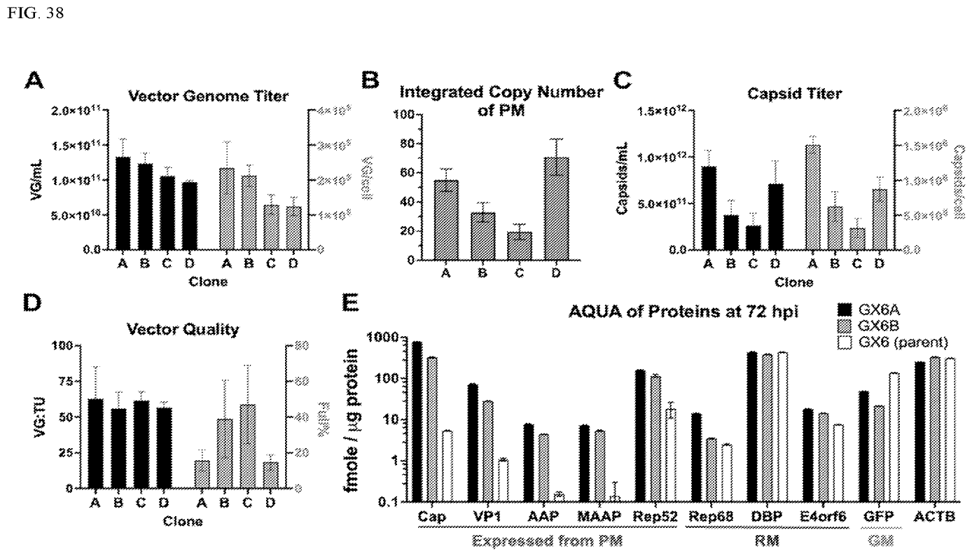

Increasing the expression of VP genes can give rise to more VP proteins and capsids and lead to higher rAAV titer. To increase expression of VP genes, additional copies of packaging module were introduced and integrated into cell line GX6. The single cell clones with high titer were isolated. The top four clones (GX6A-D) under 10D90C induction conditions generated 10⁵ VG/cell (FIG. 38A). The capsid titer, packaging copy number and the full-particle rate were different among cell clones (FIG. 38B-D). The cell clones had higher copy numbers showed higher capsid production with somewhat lower full-particle content (FIG. 38B-D). The viral proteins content, including Cap, AAP, MAAP and Rep52 for Clones A and B (GX6A and GX6B respectively) along with the parent GX6 are shown in FIG. 38E. Increased protein level on genes encoded in the PM (VP1, AAP, Rep52) was observed in the two clones compared to the parent.

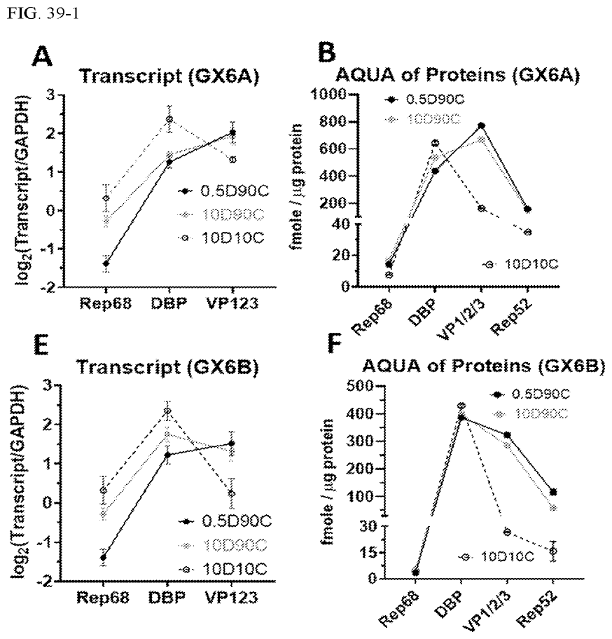

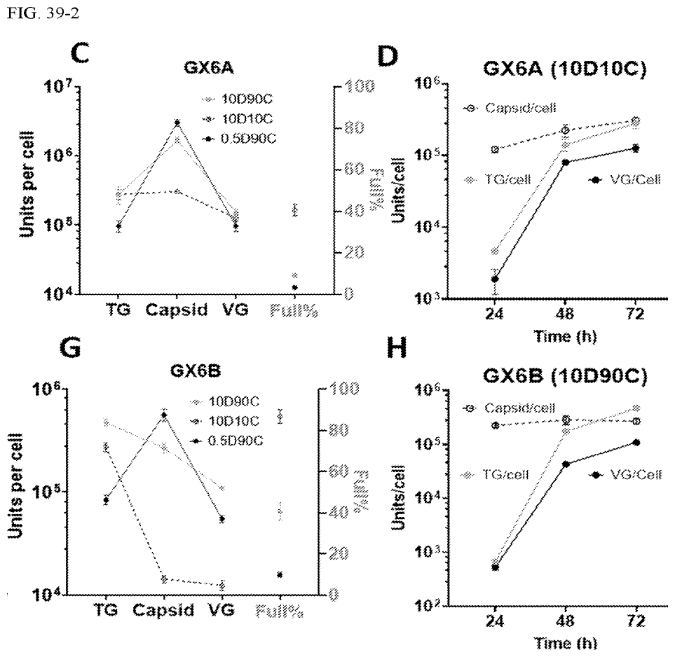

In this design, replication and helper-related genes were under TetOn control, while the capsid formation (VPs) and packaging-related genes (Rep52) were under cumate switch control. Altering induction conditions (e.g., timing of induction timing and/or inducer concentration) shifted the balance between genome replication and capsid formation, and influences product titer and productivity. The ability of tuning product titer and product quality was demonstrated by comparing the original inductions of 10D90C and 0.5D90C and a reduced cumate level of 10D10C for two cell lines. Data obtained at 72 hpi are shown in FIG. 39.

Reducing Dox concentration from 10 to 0.5 μg/mL reduced the transcript level of Rep68 and, to a lesser degree, the DBP level in both GX6A and GX6B (FIG. 39A, E). Interestingly, the reduced transcript level did not result in a reduced protein level (FIG. 39B, F). This may reflect that Rep68 protein had an additional control by the destabilization domain. Reducing cumate induction concentration from 90 to 10 g/mL lowered VP123 notably at both the transcript and protein levels in both cell lines (FIG. 39A, B, E, F).

The high abundance of VP1/2/3 proteins under high (90C) cumate induction led to very high levels of capsids in both GX6A and GX6B cells (FIG. 39C, 39G), whereas Dox induction concentration affected the total virus genome (TG) level. TG level decreased with reduced Dox level (0.5D90C) (FIG. 39C, G). At a cumate induction concentration of 90 g/mL, excess capsids resulted in high levels of empty particles. Reducing to 10 μg/mL resulted in higher full particle content (FIG. 39C, G).

### Example 3

A proteomic study revealed VP1/2/3 proteins increased rapidly upon induction, reaching levels higher than beta-actin in 24 hours. Further, a high percentage of VP1/2/3 proteins was assembled into capsids, leading to capsids rapidly accumulating to more than 10⁵ copies at 24 hpi in both GX6A and GX6B cells. In comparison, the increase of TG and VG levels lagged behind (FIGS. 39D and 39H) and did not reach a comparable level of capsids until 48 hpi. Hence, for a long period of time, there were not enough TG to be packaged into capsids. At a low dose of Dox (0.5D90C), the slower genome amplification may be the main cause of a lower VG titer and lower full particle content.

By tuning induction time profile, one can specifically delay the induction of cumate to slow down the accumulation of intracellular capsid level, thereby shifting the balance between VG titer and full particle content.

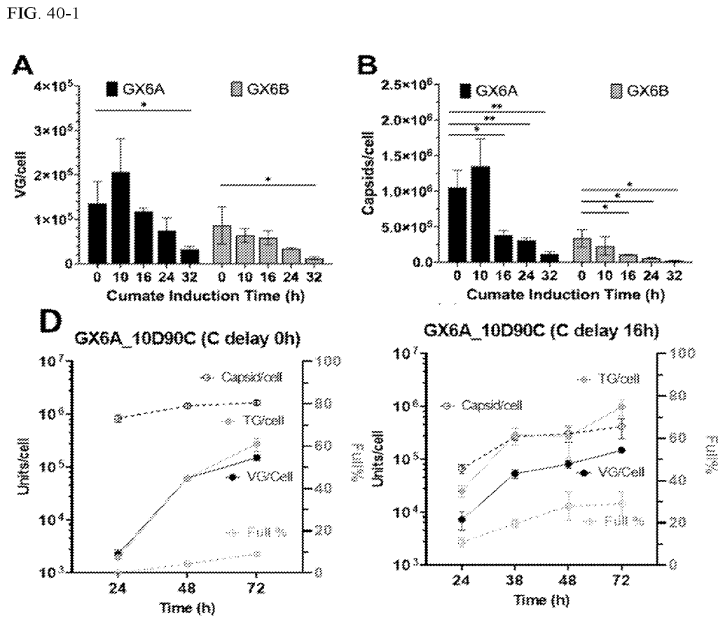

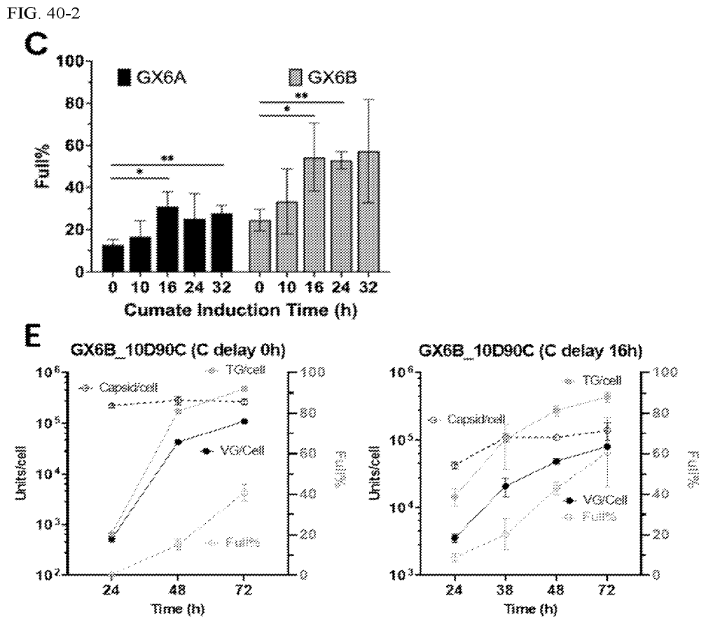

The GX6A and GX6B cells were first induced with Dox (10 μg/mL) alone at 0 hpi. Subsequently, cumate was added to culture (90 g/mL) to induce VP protein synthesis at different time points, specifically at 0, 10, 16, 24, or 32 hpi. At 72 hours post Dox induction, the cells were harvested and lysed for the measurement of the VG titer, capsid titer, and full particle content under each of the induction conditions. Delaying cumate induction for 10 hours, 16 hours, or 24 hours did not significantly decrease VG titers for either GX6A or GX6B cells (FIG. 40A). However, a significant decrease in capsid titers was observed when cumate induction was delayed by 16 hours and over (FIG. 40B). Consequently, for both GX6A and GX6B cells, delaying cumate induction by 16 hours led to a significant increase in full particle content (GX6A: from 13% to 31%, GX6B: from 25% to 54%) without a significant decrease in the VG titer (FIG. 40C).

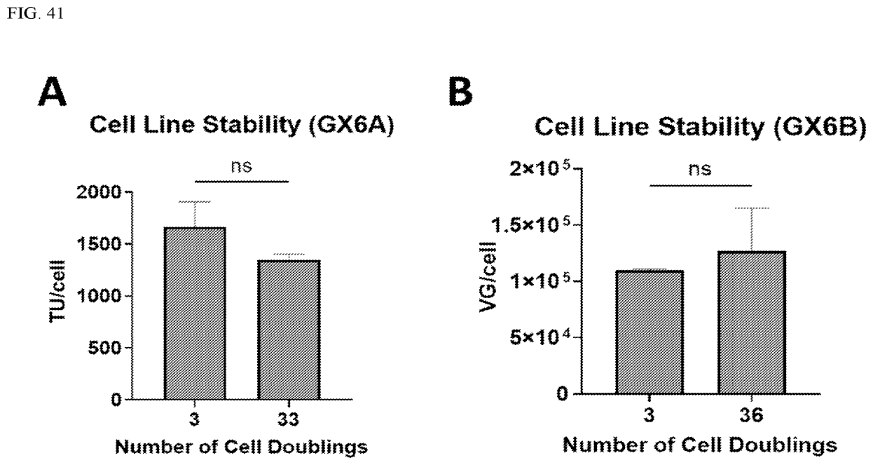

VG, TG, and capsid dynamics were further investigated with and without a 16-hour delay in cumate induction. As shown in FIGS. 40D and 40E, delaying cumate induction notably slowed down capsid formation. Capsid titers at 24 hours decreased by approximately 10-fold for GX6A cells and 5-fold for GX6B cells. In addition, an increase in TG levels at 24 hours of more than 10-fold was observed. Additionally, full particle content was improved. The temporal control of genome replication and capsid protein expression through the delay in cumate induction successfully shifted the balance between viral genomes and capsids in synthetic cells, resulting in higher full particle content. The cell line is stable in sustaining its productivity over time. After 20-30 doubling in adherent culture, the rAAV productivity maintained in the same levels (FIG. 41).

### Example 4

The GX6B cell line typically adheres when cultured. However, for large-scale culture, suspension growth is preferred over adherent cultivation. GX6B cells were thus adapted to suspension culture.

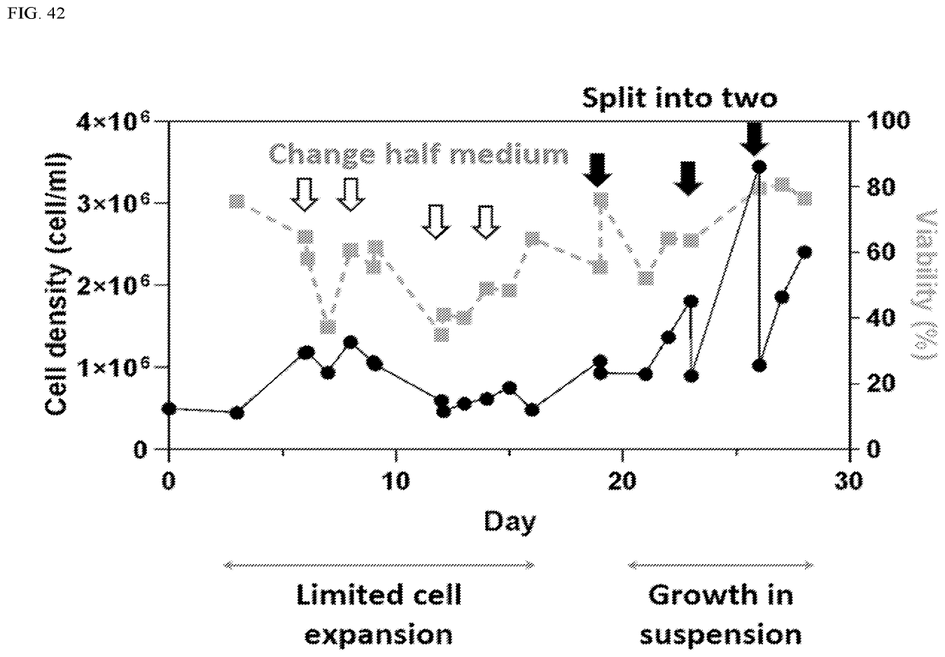

GX6B were trypsinized and transferred to shaker flasks with a 30 mL working volume in a protein-free CELER-S001 medium (BioEngine, Shanghai, People's Republic of China) at a cell concentration of 5×10⁵ cells/mL. After a period of about two weeks of static cell density with little apparent proliferation, cells began to proliferate (FIG. 42). On Day 19, GX6B cells were harvested and resuspended into two new shaker flasks and began to expand its population for building up cell stocks (named GX6Bs) (FIG. 42).

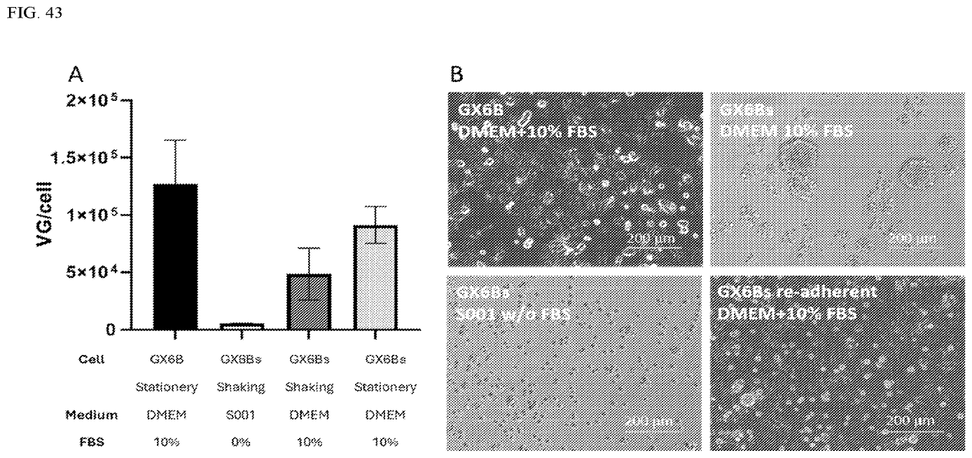

Upon induction GX6Bs produced although the productivity was somewhat lower. The rAAV productivity was mostly returned to the level of its adherent parent when the medium was changed back to adherent medium (DMEM) with 10% FBS and kept in suspension growth by placing the culture on a shaker (FIG. 43). When the culture with DMEM and 10% FBS was allowed to grow stationarily. GX6Bs reversed to grow adherently and the rAAV productivity returned to the original adherent level (FIG. 43).

### Example 5

To increase AAV genome production to enhance the AAV2 productivity and increase the full particle content, cells were modified to express HSV-1 DNA polymerase. HSV-1 DNA polymerase complex consists of the core enzyme UL30 and the processivity factor UL42. It has been reported to catalyze AAV DNA replication.

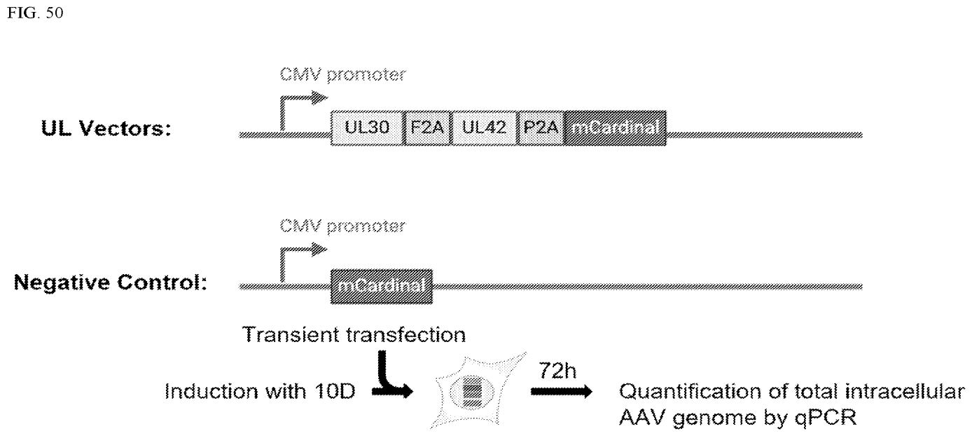

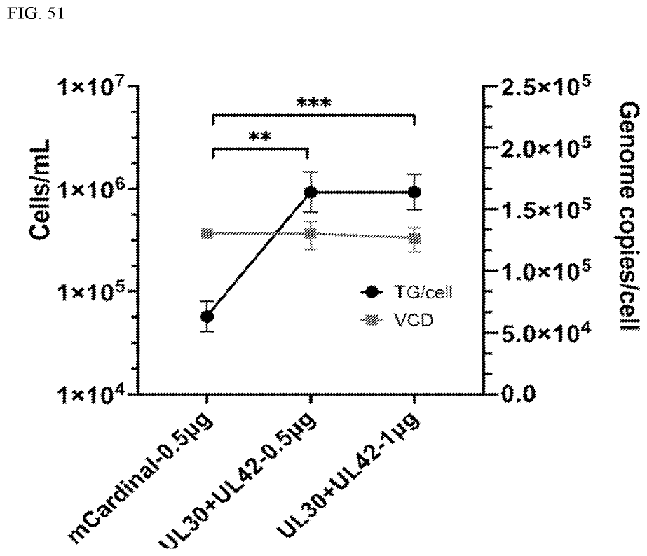

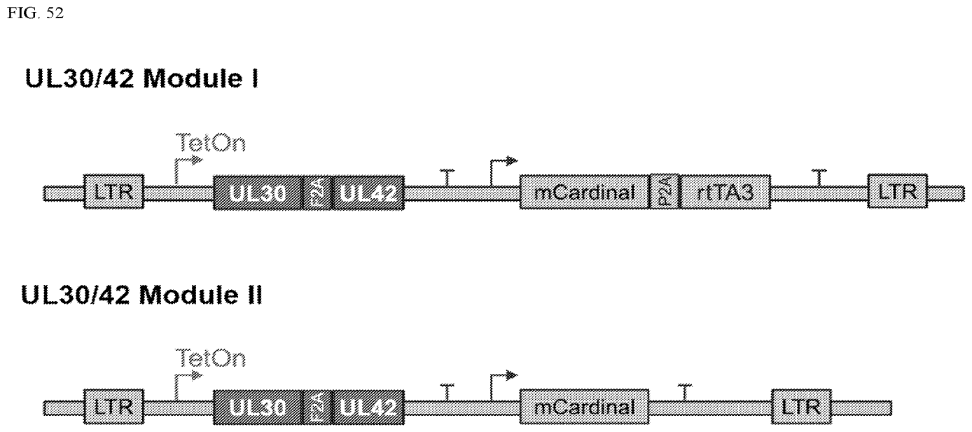

UL vector containing UL30 and UL42 genes under CMV promoter control was transfected into GX6B cells (FIG. 50). A vector containing only the reporter gene but not UL30 and UL42 was used as a negative control. The transfectant was induced with doxycycline for 72 hr. The TG titer was titrated using qRT-PCR. As shown in FIG. 51, the TG titer of GX6B transfected with either 0.5 or 1 μg of UL expression vector had approximately three-fold higher viral genome copies than the negative control (FIG. 51). The viability of GX6B did not decrease markedly upon UL30 and UL42 expression.

Two designs of Lentiviral vector, UL30/42 Module I and II, were used for integration into rAAV producer cell line for the expression of UL30 and UL42. The UL30-F2A-UL42 segment was placed in the downstream of an inducible TetOn promoter. The vector also had a constitutively expressed mCardinal reporter with or without reverse Tet transactivator gene (rtTA3) (FIG. 52).

### Example 6

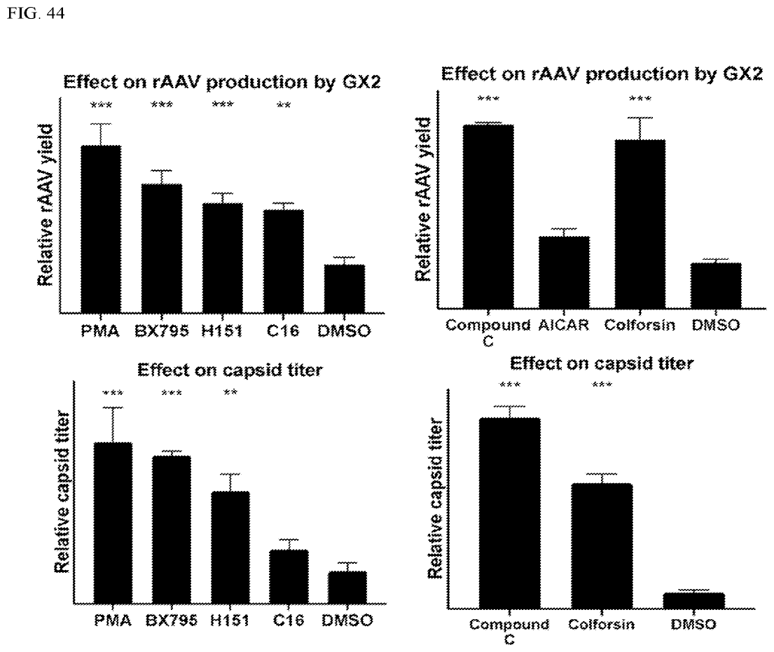

Significant increases in rAAV titer were obtained through the addition of several small molecules, PMA, BX795, H141 and C16 in GX2 cell line as compared to DMSO control (FIG. 44).

Significant increases in rAAV titer were obtained using BX795. BX795 inhibits TBK1/IKKε complex, which activates IRF3 and the following interferon responses. H151, an inhibitor of the STING-mediated DNA sensing signaling, also increased the rAAV titer. Inhibition of the dsRNA sensor, dsRNA-dependent protein kinase R (PKR), by C16 also enhanced rAAV productivity of synthetic cells.

PMA activated protein kinase C (PKC) signaling with increased NFκB and MAPK activity, which potentially stimulates cell survival and transcription activity, led to greatly increased rAAV titers.

Dorsomorphin (compound C) is an ATP-competitive inhibitor of AMPK and induces autophagy. GX2 cells treated with compound C had a three-fold increase in titer. One potential explanation is that increased ratio of AMP/ATP due to extensive ATP consumption triggers activation of AMPK signaling. This activation induces catabolic processes for energy generation while inhibits energy-consuming anabolic processes such as protein synthesis and cell growth.

Colforsin, which modulates cAMP signaling and induces autophagy, also had a positive effect on rAAV yield. Colforsin increases the cAMP level, activating protein kinase A (PKA), which further activates various proteins, including metabolic enzymes, MAPKs, and cAMP response element-binding protein (CREB). CREB activates the transcription of genes associated with cell proliferation, survival, inflammation, suppressed apoptosis, and increased metabolism. Activation of these biological processes might collectively contribute to the positive impact of Colforsin on rAAV productivity.

### Example 7

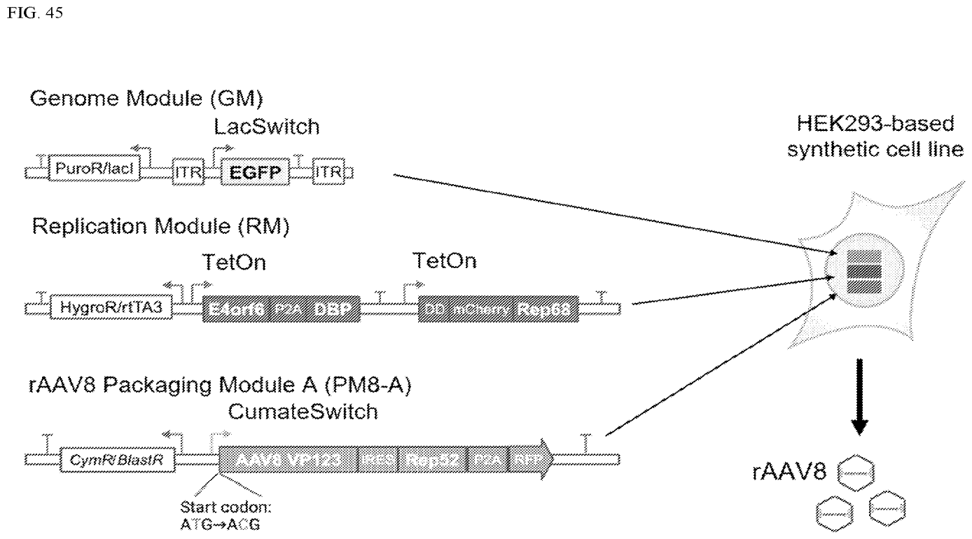

The methods and cell lines for the production of rAAV vectors can be used to produce many different serotypes of rAAV. To create an rAAV8 production cell line, the cap gene of AAV2 in the original packaging module was replaced with that of AAV8 (FIG. 45). The packaging module for rAAV8 included AAV8 intron-less cap gene (VP123) with an inefficient ACG start codon for VP1. VP123 was link to AAV2 Rep52 by internal ribosome entry site (IRES) and placed at downstream of CumateSwitch promoter. The resulting rAAV8 packaging module A was called PM8-A (FIG. 45).

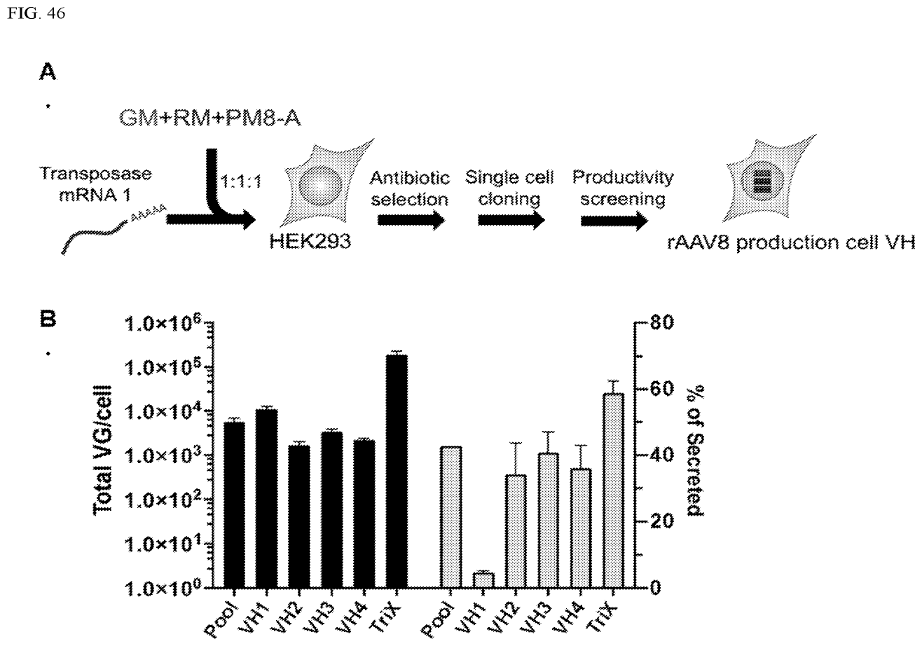

The GM, RM and PM8-A were integrated to the genome of HEK293 cells. Following antibiotic resistance selection, single cell cloning, and rAAV8 productivity screening, stable production cell lines VH were obtained (FIG. 46A). The total vector genome (VG) titer of cell lines VH1-4 following induction for 72 hours is shown in FIG. 46B. The total VG titer includes virus secreted into the media and harvested from cell lysis. Both their total VG titers and the percent of VG secreted in media were lower than the traditional rAAV8 triple plasmid transient transfection of HEK293 cells (TriX).

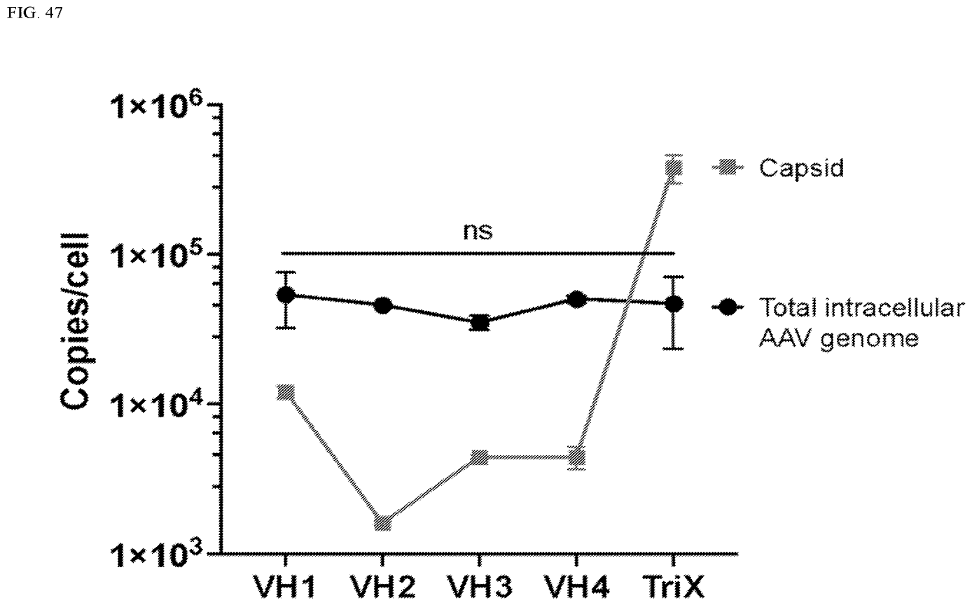

FIG. 47 shows the TG content and capsid content per cell of the four VH clones and TriX after 72 hours of induction or transfection. The capsid titer of the four VH clones was 50-fold to 100-fold lower than that of TriX (FIG. 47), suggesting that AAV8 capsid production may be limiting rAAV productivity.

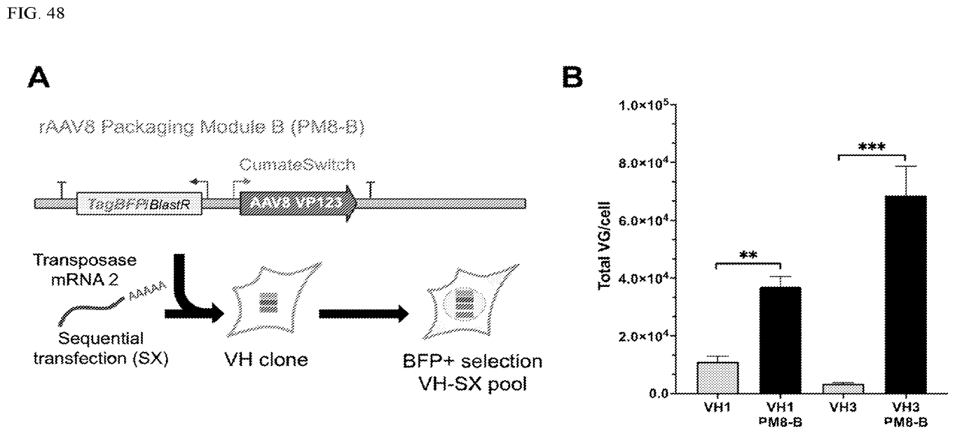

To enhance VP protein synthesis and increase capsid production, the rAAV8 packaging module B (PM8-B) was constructed. This module was designed to integrated additional copies of AAV8 VP123 to VH clones (FIG. 48). PM8-B included a cumate switch-driven AAV8 VP123 coding sequence and a TagBFP encoding blue fluorescent protein as a reporter for fluorescence-activated cell sorting (FACS) (FIG. 48A). PM8-B was integrated to VH1 and VH3 clones using a transposase as described herein. BFP positive cells were sorted into cell pools. After induction for 72 hours, the total VG titers of the PM8-B-integrated VH1 and VH3 pools were higher than their untransfected parental cells (FIG. 48B). The results showed that increasing the integrated copies of AAV8 VP123 in either VH1 or VH3 boosts rAAV8 productivity significantly (**p<0.01, ***p<0.001).

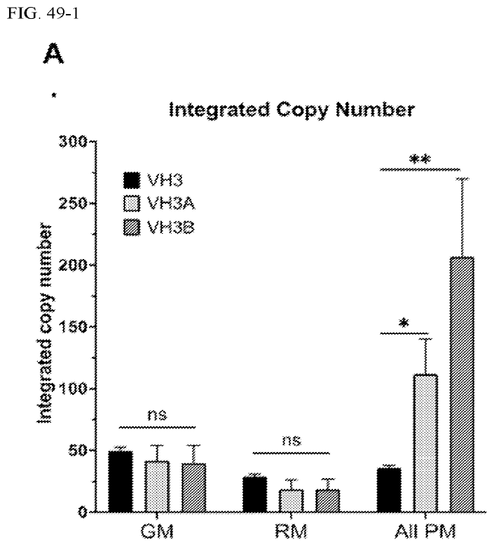

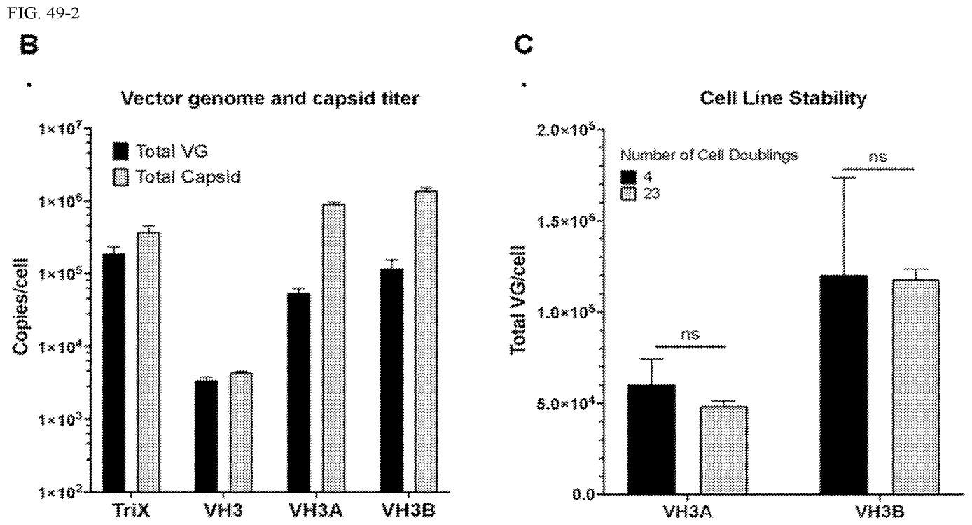

Two clones, VH3A and VH3B, were obtained from VH3 PM8-B cell pool. Both clones had more copies of PM8 integrated into cell genome (including both PM8-A and PM8-B) than the parent VH3, while the copy number of GM and RM remained at similar levels as VH3 (FIG. 49A).

Clones VH3A and VH3B produced more capsids than their parental clone VH3 (FIG. 49B). Moreover, the capsid titer of both clone VH3A and VH3B was comparable to that of TriX (FIG. 49B). With the increased capsid titer, the total VG titer of clone VH3B reached 1.2×10⁵ copies per cell, which is about 20-fold higher than that of its parental clone VH3 (FIG. 49B).

The cell lines VH3A and VH3B were found to stably produce rAAV8. The total VG titer of both VH3A and VH3B was sustained after approximately 20 cell doublings (FIG. 49C).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A stable mammalian cell line comprising:
a first polynucleotide encoding a first AAV inverted terminal repeat (ITR) sequence, a second ITR sequence, and a first promoter, wherein the first promoter is not expressed in the stable mammalian cell line and wherein the first promoter is a tissue-specific promoter or a first inducible promoter.

2. (canceled)

3. (canceled)

4. The stable mammalian cell line of claim 1, further comprising a second polynucleotide encoding a repressor sequence wherein the first inducible promoter is operably linked to the repressor.

5. (canceled)

6. The stable mammalian cell line of claim 1, wherein the first promoter is located between the first ITR sequence and the second ITR sequence.

7. The stable mammalian cell line of claim 1, wherein the first polynucleotide encodes a gene of interest (GOI) operably linked to the first promoter.

8. (canceled)

9. (canceled)

10. The stable mammalian cell line of claim 1, wherein the first polynucleotide further comprises a selectable marker.

11. (canceled)

12. (canceled)

13. The stable mammalian cell line of claim 1, further comprising:
a third polynucleotide encoding an AAV large replicate (Rep) protein, wherein the third polynucleotide is operably linked to a second inducible promoter;
a fourth polynucleotide encoding an adenovirus (Ad) E4orf6, wherein the fourth polynucleotide is operably linked to a third inducible promoter; and
a fifth polynucleotide encoding an Ad DNA binding protein (DBP), wherein the fifth polynucleotide is operably linked to a fourth inducible promoter.

14-24. (canceled)

25. A stable mammalian cell line, comprising:
a first polynucleotide encoding an AAV large replicate (Rep) protein, wherein the first polynucleotide is operably linked to a first inducible promoter;
a second polynucleotide encoding an adenovirus (Ad) E4orf6, wherein the second polynucleotide is operably linked to a second inducible promoter;
a third polynucleotide encoding an Ad DNA binding protein (DBP), wherein the third polynucleotide is operably linked to a third inducible promoter;
a fourth polynucleotide encoding an AAV capsid protein, wherein the fourth polynucleotide is operably linked to a fourth inducible promoter; and
a fifth polynucleotide encoding an AAV small Rep protein, wherein the fifth polynucleotide is operably linked to a fifth inducible promoter.

26. The stable mammalian cell line of claim 25, further comprising a sixth polynucleotide encoding a first AAV inverted terminal repeat (ITR) sequence.

27-30. (canceled)

31. The stable mammalian cell line of claim 25, wherein the first inducible promoter, the second inducible promoter, and the third inducible promoter are induced by a common stimulus.

32. The stable mammalian cell line of claim 31, wherein the common stimulus comprises doxycycline.

33. The stable mammalian cell line of claim 25, wherein the first polynucleotide is tagged with a destabilization domain.

34. The stable mammalian cell line of claim 25 wherein:
the fourth inducible promoter comprises a cumate-inducible promoter; and
the fifth inducible promoter comprises a cumate-inducible promoter.

35-37. (canceled)

38. The stable mammalian cell line of claim 25, wherein at least one inducible promoter comprises a doxycycline-inducible promoter, a mifepristone-inducible promoter, a lactose-inducible promoter, or a cumate-inducible promoter.

39. The stable mammalian cell line of claim 25, further comprising an exogenous DNA polymerase.

40-43. (canceled)

44. A method of using the stable mammalian cell line of claim 1, the method comprising contacting the mammalian cell line with an inducer of one or more of the inducible promoters, generating AAV particles, and measuring a concentration of infectious recombinant AAV particles.

45-53. (canceled)

54. The method of claim 44, wherein the inducer comprises lactose, IPTG, doxycycline, tetracycline, mifepristone, or cumate.

55-57. (canceled)

58. The method of claim 44, wherein the method comprises contacting the cell line with more than one inducer.

59-65. (canceled)

66. A method of using the stable mammalian cell line of claim 1, wherein the method comprises:
(i) contacting the cell with a second inducer, wherein the second inducer induces expression of the second inducible promoter, contacting the cell with a third inducer, wherein the third inducer induces expression of the third inducible promoter, or a fourth inducer, wherein the fourth inducer induces expression of the fourth inducible promoter;
(ii) contacting the cell with a first inducer, wherein the first inducer induces expression of the first inducible promoter.

67. The method of claim 66, wherein the method comprises contacting the cell with the second inducer, the third inducer, and the fourth inducer.

68. The method of claim 66, wherein the cell is contacted with the first inducer at least 10 hours after the cell is contacted with the second inducer, the third inducer, and/or the fourth inducer.
